(19) United States
(12) Patent Application Publication
(10) Pub. No.: US 2026/0358706 A1
(43) Pub. Date: Nov. 12, 2026

(54) RADIATION-SENSITIVE COMPOSITION, RESIST-PATTERN-FORMING METHOD, RADIATION-SENSITIVE ACID GENERATOR AND POLYMER

(71) Applicant: JSR CORPORATION, Tokyo (JP)

(72) Inventors: Takuhiro TANIGUCHI, Tokyo (JP); Kazuya KIRIYAMA, Tokyo (JP); Natsuko KINOSHITA, Tokyo (JP); Katsuaki NISHIKORI, Tokyo (JP)

(73) Assignee: JSR CORPORATION, Tokyo (JP)

(21) Appl. No.: 19/110,507

(22) PCT Filed: Jul. 18, 2023
(86) PCT No.: PCT/JP2023/026224

§ 371 Date: Mar. 11, 2025

(30) Foreign Application Priority Data
Sep. 13, 2022 (JP) 2022-145580
Feb. 17, 2023 (JP) 2023-023704

(51) Int. Cl. G03F 7/004; C07C 309/12; C07C 309/58; C07C 321/28; C07D 317/08; C07D 317/24; C08F 220/18; C08F 220/40; G03F 7/00; G03F 7/039; G03F 7/20

(52) U.S. Cl. CPC G03F 7/0045; C07C 309/12; C07C 309/58; C07C 321/28; C07D 317/08; C07D 317/24; C08F 220/1806; C08F 220/1807; C08F 220/1808; C08F 220/1809; C08F 220/1812; C08F 220/1818; C08F 220/40; G03F 7/0397; G03F 7/2004; G03F 7/70033; C08F 2800/10

(57) ABSTRACT

The radiation-sensitive composition contains a polymer having an acid-releasable group and a compound represented by formula (1). In the formula (1), R⁵ is a group formed by removing (m+n+2) hydrogen atoms from a monocyclic or condensed cyclic aromatic hydrocarbon ring having r carbon atoms. L⁻ represents —SO₃⁻ or —COO⁻. r is an integer of 6 to 14. A¹ represents a single bond, —O—, —S—, or —NR³—. R¹ represents a hydrogen atom or a monovalent organic group. X¹ represents a chlorine atom, a bromine atom, or an iodine atom. m is an integer of 1 to (r−2). n is an integer of 0 to (r−3). R² represents a substituted or unsubstituted monovalent hydrocarbon group. M⁺ represents a monovalent cation.

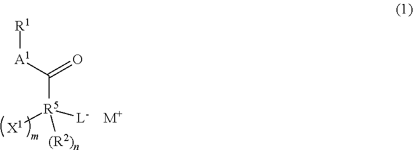

### TECHNICAL FIELD

### Cross-Reference to Related Applications

The present application claims priority from Japanese Patent Application No. 2022-145580 filed on Sep. 13, 2022, and Japanese Patent Application No. 2023-23704 filed on Feb. 17, 2023, the entirety of these applications is incorporated herein by reference.

The present disclosure relates to a radiation-sensitive composition, to a method for forming a resist pattern (hereinafter may also be referred to as a “resist pattern formation method”), to a radiation-sensitive acid-generator, and to a polymer.

### BACKGROUND ART

Fine circuit patterns of a semiconductor element are formed through a photolithographic technique by use of a resist composition. In a typical procedure, film of a resist composition is irradiated with radiation (i.e., exposure to light) by the mediation of a mask pattern, to thereby generate an acid. Through chemical reaction involved in the generated acid, a difference in solubility in a developer between the light-exposed part and the light-unexposed part (i.e., dissolution contrast) is provided. Thereafter, the irradiated pattern is developed, to thereby form a resist pattern on a substrate. Hitherto, various resist compositions have been proposed for forming fine resist patterns by employing a photolithographic technique (see, for example, Patent Documents 1 to 3). Patent Documents 1 to 3 disclose resist compositions each containing a base polymer and a sulfonium salt or an iodonium salt as a radiation-sensitive acid-generating agent.

In order to control or suppress diffusion of an acid to the light-unexposed part so as to enhance dissolution contrast between the light-exposed part and the light-unexposed part, a quencher (acid diffusion control agent) is added to a resist composition (see, for example, Patent Document 4). Patent Document 4 discloses a resist composition containing a base polymer and a quencher, which is an onium salt formed of an anion having a benzene ring to which iodine is bound, and a sulfonium cation.

### PRIOR ART DOCUMENTS

### Patent Documents

Patent Document 1: Japanese Patent Application Laid-Open (kokai) No. 2018-025789
Patent Document 2: Japanese Patent Application Laid-Open (kokai) No. 2018-005224
Patent Document 3: Japanese Patent Application Laid-Open (kokai) No. 2018-159744
Patent Document 4: Japanese Patent Application Laid-Open (kokai) No. 2017-219836

### SUMMARY OF THE INVENTION

### Problems to be Solved by the Invention

In photolithographic techniques, process shrink is in progress by employing, for example, short-wave radiation (e.g., ArF excimer laser light) or liquid immersion lithography, in which light exposure is conducted in a state where a space between a lens of an exposure device and the resist film is filled with a liquid medium. Also, as a next-generation technique, lithography employing radiation of a shorter wavelength (e.g., electron beam, X-ray, or extreme ultraviolet (EUV)) has been investigated. In development of such next-generation techniques, demand has arisen for enhancing performances including radiation sensitivity and LWR (line width roughness); i.e., an index for variation in line width of a resist pattern, to a level equal to or higher than a conventional level.

In further down-scaling of resist patterns, only a slight variation in process conditions (e.g., conditions for exposure and development) readily causes morphological problems, occurrence of failures, and the like which are involved in resist patterns. Thus, the radiation-sensitive composition must have such wide allowance that can accept slight variation in process conditions. That is, there is demand for wide ranges of process conditions which ensure formation of a pattern having no bridge defects and collapses in the resist pattern formation step (hereinafter, such a range may also be referred to as a “process window”).

The present disclosure has been made in view of the aforementioned problems. Thus, a main object of the disclosure is to provide a radiation-sensitive composition that can provide a resist film exhibiting high sensitivity, a wide process window, and excellent LWR performance. Another object is to provide a resist pattern formation method.

### Means for Solving the Problems

The present inventors have found that the aforementioned objects can be attained by a radiation-sensitive composition containing a compound having a particular onium salt structure. More specifically, the present disclosure provides the following means.

In one mode of the present disclosure, there is provided a radiation-sensitive composition containing a polymer having an acid-releasable group, and a compound represented by the following formula (1).

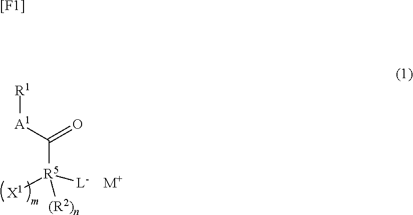

(In the formula (1), R⁵ is a group formed by removing (m+n+2) hydrogen atoms from a monocyclic or condensed cyclic aromatic hydrocarbon ring having r carbon atoms; among carbon atoms forming R⁵, the carbon atom to which group “—CO-A¹-R¹” is bound and the carbon atom to which L⁻ is bound are located adjacent to each other; L⁻ represents —SO₃⁻ or —COO⁻; and r is an integer of 6 to 14; A¹ represents a single bond, —O—, —S—, or —NR³—; R³ represents a hydrogen atom or a monovalent organic group; R¹ represents a hydrogen atom or a monovalent organic group; X¹ represents a chlorine atom, a bromine atom, or an iodine atom; m is an integer of 1 to (r−2); when m is 2 or greater, a plurality of X¹s are identical to or different from one another; n is an integer of 0 to (r−3); R² represents a substituted or unsubstituted monovalent hydrocarbon group; when n is 2 or greater, a plurality of R²s are identical to or different from one another; m+n is equal to or less than (r−2); and M⁺ represents a monovalent cation.)

In another mode of the present disclosure, there is provided a radiation-sensitive composition containing a polymer having a partial structure represented by the following formula (2).

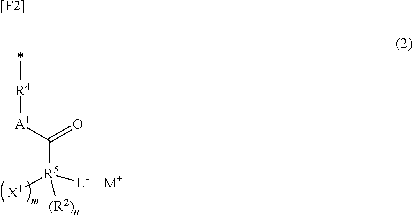

(In the formula (2), R⁵ is a group formed by removing (m+n+2) hydrogen atoms from a monocyclic or condensed cyclic aromatic hydrocarbon ring having r carbon atoms; among carbon atoms forming R⁵, the carbon atom to which group “—CO-A¹-R¹” is bound and the carbon atom to which L⁻ is bound are located adjacent to each other; L⁻ represents —SO₃⁻ or —COO⁻; and r is an integer of 6 to 14; A¹ represents a single bond, —O—, —S—, or —NR³—; R³ represents a hydrogen atom or a monovalent organic group; R⁴ represents a divalent organic group; X¹ represents a chlorine atom, a bromine atom, or an iodine atom; m is an integer of 1 to (r−2); when m is 2 or greater, a plurality of X¹s are identical to or different from one another; n is an integer of 0 to (r−3); R² represents a substituted or unsubstituted monovalent hydrocarbon group; when n is 2 or greater, a plurality of R²s are identical to or different from one another; m+n is equal to or less than (r−2); “*” represents a chemical bond; and M⁺ represents a monovalent cation.)

In still another mode of the present disclosure, there is provided a resist pattern formation method, the method including a step of forming a resist film on a substrate film by use of the aforementioned radiation-sensitive composition; a step of exposing the resist film to light; and a step of developing the light-exposed resist film.

In yet another mode of the present disclosure, there is provided a radiation-sensitive acid-generator represented by the aforementioned formula (1). Also, in further another mode of the present disclosure, there is provided a polymer having a partial structure represented by the aforementioned formula (2).

### Advantageous Effects of the Invention

According to the present disclosure, there can be yielded a radiation-sensitive composition exhibiting a high sensitivity to radiation, a wide process window, and excellent LWR performance.

### MODES FOR CARRYING OUT THE INVENTION

Hereinafter, the matters in relation to carrying out of the present disclosure will be described in detail. As used herein, the numerical range with an expression of “X to Y” refers to a numerical range that includes X and Y placed before and after “to” as a lower limit value and an upper limit value, respectively. The term “structural unit” refers to a unit mainly formed of a main chain structure, wherein at least two of the structural units are included in the main chain structure. Typically, the structural unit is a monomer unit.

### <<Radiation-Sensitive Composition>>

The radiation-sensitive composition of the present disclosure (hereinafter may also be referred to as “the present composition”) contains a compound having, in the molecule thereof, an onium salt structure which is derived an acid anhydride having an aromatic hydrocarbon ring to which at least one halogen atom selected from the group consisting of a chlorine atom, a bromine atom, and an iodine atom is bound. Specific embodiments of the present composition include the following radiation-sensitive compositions [1] and [2].

Radiation-sensitive composition [1]: a radiation-sensitive composition containing a polymer having an acid-releasable group, and a compound represented by the following formula (1).

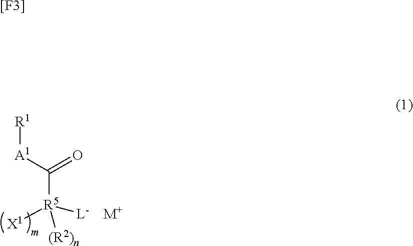

(In the formula (1), R⁵ is a group formed by removing (m+n+2) hydrogen atoms from a monocyclic or condensed cyclic aromatic hydrocarbon ring having r carbon atoms; among carbon atoms forming R⁵, the carbon atom to which group “—CO-A¹-R¹” is bound and the carbon atom to which L⁻ is bound are located adjacent to each other; L⁻ represents —SO₃⁻ or —COO⁻; and r is an integer of 6 to 14; A¹ represents a single bond, —O—, —S—, or —NR³—; R³ represents a hydrogen atom or a monovalent organic group; R¹ represents a hydrogen atom or a monovalent organic group; X¹ represents a chlorine atom, a bromine atom, or an iodine atom; m is an integer of 1 to (r−2); when m is 2 or greater, a plurality of X¹s are identical to or different from one another; n is an integer of 0 to (r−3); R² represents a substituted or unsubstituted monovalent hydrocarbon group; when n is 2 or greater, a plurality of R²s are identical to or different from one another; m+n is equal to or less than (r−2); and M⁺ represents a monovalent cation.)

Radiation-sensitive composition [2]: a radiation-sensitive composition containing a polymer having a partial structure represented by the following formula (2).

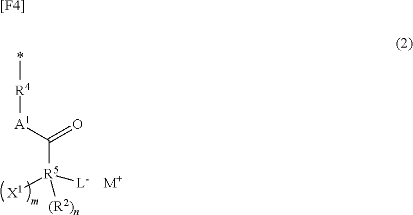

(In the formula (2), R⁵ is a group formed by removing (m+n+2) hydrogen atoms from a monocyclic or condensed cyclic aromatic hydrocarbon ring having r carbon atoms; among carbon atoms forming R⁵, the carbon atom to which group “—CO-A¹-R¹” is bound and the carbon atom to which L⁻ is bound are located adjacent to each other; L⁻ represents —SO₃⁻ or —COO⁻; and r is an integer of 6 to 14; A¹ represents a single bond, —O—, —S—, or —NR³—; R³ represents a hydrogen atom or a monovalent organic group; R⁴ represents a divalent organic group; X¹ represents a chlorine atom, a bromine atom, or an iodine atom; m is an integer of 1 to (r−2); when m is 2 or greater, a plurality of X¹s are identical to or different from one another; n is an integer of 0 to (r−3); R² represents a substituted or unsubstituted monovalent hydrocarbon group; when n is 2 or greater, a plurality of R²s are identical to or different from one another; m+n is equal to or less than (r−2); “*” represents a chemical bond; and M⁺ represents a monovalent cation.)

When L⁻ in the aforementioned formula (1) or (2) is —SO₃⁻, each of the radiation-sensitive compositions [1] and [2] contains a compound which generates sulfonic acid upon exposure to light. The sulfonic acid generated in the composition upon light exposure may be a low-molecular compound (i.e., a compound different from a polymer) or a polymer having a sulfonic acid group. When L⁻ in the aforementioned formula (1) or (2) is —COO⁻, each of the radiation-sensitive compositions [1] and [2] contains a compound which generates carboxylic acid upon exposure to light. The carboxylic acid generated in the composition upon light exposure may be a low-molecular compound or a polymer having a carboxylic acid group. In the description below, the case where L⁻ in the aforementioned formula (1) or (2) is —SO₃⁻ is referred to as a “first radiation-sensitive composition”, and the case where L⁻ in the aforementioned formula (1) or (2) is —COO⁻ is referred to as a “second radiation-sensitive composition”. Next, the first and second radiation-sensitive compositions will be described in detail.

### [First Radiation-Sensitive Composition]

The first radiation-sensitive composition contains a compound in which L⁻ in the aforementioned formula (1) or (2) is —SO₃⁻.

In the aforementioned formulas (1) and (2), examples of the aromatic hydrocarbon ring forming R⁵ include a benzene ring, a naphthalene ring, an anthracene ring, and a phenanthrene ring. From the viewpoints of ease of synthesizing the compound and availability of starting materials, the aromatic hydrocarbon ring forming R⁵ is preferably, among others, a benzene ring or a naphthalene ring, with a benzene ring being more preferred. Notably, when the aromatic hydrocarbon ring forming R⁵ is a condensed ring, no particular limitation is imposed on the bonding position of X¹ in the condensed ring. In other words, X¹ in the aforementioned formula (1) or (2) may be bound to one single ring having L⁻ among a plurality of monocycles forming the condensed ring, or different rings. Similarly, R² in the aforementioned formula (1) or (2) may be bound to one single ring having L⁻ among a plurality of monocycles forming the condensed ring, or different rings.

Preferred specific embodiments of the first radiation-sensitive composition include the following first and second embodiments.

[First embodiment] a radiation-sensitive composition which contains a polymer having an acid-releasable group, and a compound represented by the following formula (1b) (hereinafter may also be referred to as “compound (CB)”).

[Second embodiment] a radiation-sensitive composition which contains a polymer having a partial structure represented by the following formula (2b) (hereinafter may also be referred to as “polymer (PB)”).

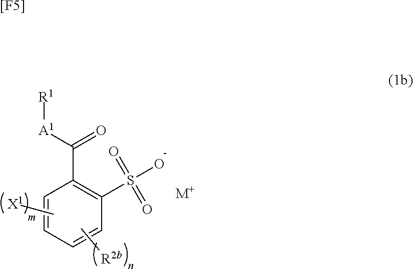

(In the formula (1b), A¹ represents a single bond, —O—, —S—, or —NR³—; R³ represents a hydrogen atom or a monovalent organic group; R¹ represents a hydrogen atom or a monovalent organic group; X¹ represents a chlorine atom, a bromine atom, or an iodine atom; m is an integer of 1 to 4; when m is ≥2, a plurality of X¹s are identical to or different from one another; n is an integer of 0 to 3; when n is 1, R^{2b} is a substituted or unsubstituted monovalent hydrocarbon group; when n is ≥2, a plurality of R^{2b} are independently a substituted or unsubstituted monovalent hydrocarbon group, or two Ribs are combined to form a ring structure formed with a carbon atom to which two Ribs are bonded; m+n is 4 or less; and M⁺ represents a monovalent cation.)

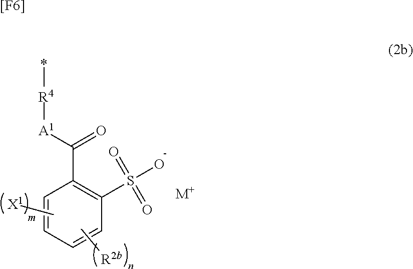

(In the formula (2b), A¹ represents a single bond, —O—, —S—, or —NR³—; R³ represents a hydrogen atom or a monovalent organic group; R⁴ represents a divalent organic group; X¹ represents a chlorine atom, a bromine atom, or an iodine atom; m is an integer of 1 to 4; when m is ≥2, a plurality of X¹s are identical to or different from one another; n is an integer of 0 to 3; when n is 1, R^{2b} is a substituted or unsubstituted monovalent hydrocarbon group; when n is ≥2, a plurality of R^{2b} are independently a substituted or unsubstituted monovalent hydrocarbon group, or two Ras are combined to form a ring structure formed with a carbon atom to which two Ribs are bonded; m+n is 4 or less; “*” represents a chemical bond; and M⁺ represents a monovalent cation.)

Hereinafter, a radiation-sensitive composition of the first embodiment according to the first radiation-sensitive composition (hereinafter may also be referred to as a “first composition”) will be described. Then, a radiation-sensitive composition of the second embodiment according to the second radiation-sensitive composition (hereinafter may also be referred to as a “second composition”) will be described.

As used herein, the term “hydrocarbon group” encompasses a chain hydrocarbon group, an alicyclic hydrocarbon group, and an aromatic hydrocarbon group. The term “chain hydrocarbon group” refers to a linear-chain hydrocarbon group or a branched hydrocarbon group including one which is composed of only a chain structure and no ring structure. However, the chain hydrocarbon group may be saturated or unsaturated. The term “alicyclic hydrocarbon group” refers to a hydrocarbon group which contains only an alicyclic hydrocarbon moiety as a ring structure and contains no aromatic ring structure. However, the alicyclic hydrocarbon group is not necessarily formed only of an alicyclic hydrocarbon moiety and may contain a chain structure as a partial structure. The term “aromatic hydrocarbon group” refers to a hydrocarbon group which contains an aromatic ring structure as a ring structure. However, the aromatic hydrocarbon group is not necessarily formed only of an aromatic ring structure and may contain a chain structure or an alicyclic hydrocarbon moiety as a partial structure. The term “organic group” refers to an atomic group formed by removing any hydrogen atom from a carbon-containing compound (i.e., an organic compound). The term “(meth)acryl” is a term encompassing “acryl” and “methacryl”.

The expression “substituted or unsubstituted p-valent hydrocarbon group (p is an integer of ≥1)” encompasses a p-valent hydrocarbon group (i.e., an unsubstituted p-valent hydrocarbon group) and a group formed by removing p hydrogen atoms from a hydrocarbon structure moiety of a hydrocarbon group having a substituent. Examples of the substituted or unsubstituted p-valent hydrocarbon group include an alkyl group and a fluoroalkyl group (corresponding to the case of p=1), and an alkanediyl group and a fluoroalkanediyl group (corresponding to the case of p=2). Among them, a fluoroalkyl group corresponds to a “substituted monovalent hydrocarbon group”, and a fluoroalkanediyl group corresponds to a “substituted divalent hydrocarbon group”. Such a feature similarly applies to other groups with the expression of “substituted or unsubstituted”.

### <First Composition>

The first composition of the present disclosure contains a polymer having an acid-releasable group (hereinafter may also be referred to as a “polymer (A)”), and a compound (CB). So long as the effects of the present disclosure are not impaired, the first composition may further contain an optional component other than the polymer (A) and the compound (CB). Examples of the optional component include a radiation-sensitive acid-generating agent (except for compound (CB)), hereinafter may also be referred to as an “additional acid-generating agent”), an acid diffusion control agent, a solvent, and a high-fluorine content polymer. These components will next be described in detail.

### [Polymer (A)]

The acid-releasable group present in the polymer (A) is a group which substitutes a hydrogen atom of an acidic group such as a carboxy group or a hydroxy group and which is released by the action of acid. In the first composition, containing a polymer having an acid-releasable group, an acid is generated through light exposure of the first composition, and the acid-releasable group is released by the acid, to thereby form an acidic group. As a result, solubility of a polymer component in a developer can be modified. Thus, the first composition can be provided with suitable lithographic characteristics. From the viewpoint of achieving sufficiently high solubility of the polymer (A) in a developer, the polymer (A) preferably includes a structural unit having an acid-releasable group (hereinafter may also be referred to as a “structural unit (I)”).

### Structural Unit (I)

So long as the structural unit (I) has an acid-releasable group, no particular limitation is imposed on the structures of the other moieties of the structural unit (I). Examples of the structural unit (I) include structural units represented by the following formula (i-1) (hereinafter may also be referred to as “structural units (1-1)”), structural units represented by the following formula (i-2) (hereinafter may also be referred to as “structural units (1-2)”), and structural units represented by the following formula (i-3) (hereinafter may also be referred to as “structural units (I-3)”).

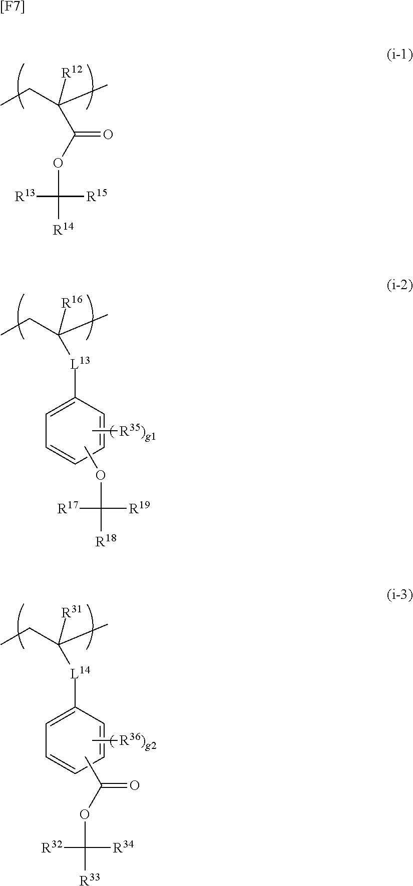

(In the formula (i-1), R¹² represents a hydrogen atom, a fluorine atom, a methyl group, or a trifluoromethyl group; R¹³ is a hydrogen atom or a C1 to C20 monovalent hydrocarbon group; each of R¹⁴ and R¹⁵ independently represents a C1 to C20 monovalent hydrocarbon group, or R¹⁴ and R¹⁵ are linked to form a C3 to C20 alicyclic structure including the carbon atom to which R¹⁴ and R¹⁵ are bound; when R¹³ is a hydrogen atom, at least one of R¹⁴ and R¹⁵ represents a monovalent unsaturated hydrocarbon group, R¹⁴ and R¹⁵ are linked to form a C3 to C20 unsaturated alicyclic structure including the carbon atom to which R¹⁴ and R¹⁵ are bound; and hydrogen atoms of each of R¹³, R¹⁴, and R¹⁵ may be partially or totally substituted with a halogen atom;
in the formula (i-2), R¹⁶ represents a hydrogen atom, a fluorine atom, a methyl group, or a trifluoromethyl group; L¹³ represents a single bond, —COO—, or —CONH—; each of R¹⁷, R¹⁸, and R¹⁹ independently represents a hydrogen atom, a C1 to C20 monovalent hydrocarbon group, or a C1 to C20 monovalent oxyhydrocarbon group; R³⁵ represents a monovalent substituent; g1 is an integer of 0 to 4; and hydrogen atoms of each of R¹⁷, R¹⁸, and R¹⁹ may be partially or totally substituted with a halogen atom; and
in the formula (i-3), R³¹ represents a hydrogen atom, a fluorine atom, a methyl group, or a trifluoromethyl group; L¹⁴ represents a single bond, —COO—, or —CONH—; each of R³², R³³, and R³⁴ independently represents a hydrogen atom, a C1 to C20 monovalent hydrocarbon group, or a C1 to C20 monovalent oxyhydrocarbon group; R³⁶ represents a monovalent substituent; g2 is an integer of 0 to 4; and hydrogen atoms of each of R³², R³³, and R³⁴ may be partially or totally substituted with a halogen atom.)

In the aforementioned formula (i-1), R¹² is preferably a hydrogen atom or a methyl group, more preferably a methyl group, from the viewpoint of co-polymerizability of a monomer providing the structural unit (1-1). In the aforementioned formula (i-2), from the viewpoint of co-polymerizability of a monomer providing the structural unit (1-2), R¹⁶ is preferably a hydrogen atom. Similarly, R³¹ in the aforementioned formula (i-3) is preferably a hydrogen atom.

Examples of the C1 to C20 monovalent hydrocarbon group represented by R¹³, R¹⁴, R¹⁵, R¹⁷, R¹⁸, R¹⁹, R³², R³³, or R³⁴ include a C1 to C20 monovalent chain hydrocarbon group, a C3 to C20 monovalent alicyclic hydrocarbon group, and a C6 to C20 monovalent aromatic hydrocarbon group.

Examples of the C1 to C20 monovalent chain hydrocarbon group include alkyl groups such as methyl, ethyl, n-propyl, i-propyl, n-butyl, and t-butyl; alkenyl groups such as ethenyl, propeny, and butenyl; and alkynyl groups such as ethynyl, propynyl, and butynyl. Among them, the C1 to C20 monovalent chain hydrocarbon group represented by R¹³ or the like is preferably an alkyl group, more preferably a C1 to C4 alkyl group.

Examples of the C3 to C20 monovalent alicyclic hydrocarbon group include monovalent monocyclic alicyclic saturated hydrocarbon groups such as cyclopentyl, cyclohexyl, methylcyclopentyl, ethylcyclopentyl, methylcyclohexyl, and ethylcyclohexyl; monovalent monocyclic alicyclic unsaturated hydrocarbon groups such as cyclopentenyl, cyclohexenyl, methylcyclopentenyl, and methylcyclohexenyl; monovalent polycyclic alicyclic saturated hydrocarbon groups such as norbornyl, adamantyl, and tricyclodecyl; and monovalent polycyclic alicyclic unsaturated hydrocarbon groups such as norborenyl, tricyclodecenyl, indenyl, and indanyl.

Examples of the C6 to C20 monovalent aromatic hydrocarbon group include aryl groups such as phenyl, tolyl, xylyl, mesityl, vinylphenyl, naphthyl, methyl naphthyl, anthryl, and methylanthryl; and aralkyl groups such as benzyl, phenethyl, naphtylmethyl, and anthrylmethyl.

Examples of the C3 to C20 alicyclic structure including the carbon atom to which R¹⁴ and R¹⁵ are bound, the structure being formed by linking R¹⁴ and R¹⁵, include monocyclic saturated alicyclic structures such as a cyclopropane structure, a cyclobutane structure, a cyclopentane structure, a cyclohexane structure, a cycloheptane structure, a cyclooctane structure; monocyclic unsaturated alicyclic structures such as a cyclopentene structure and a cyclohexene structure; and polycyclic alicyclic structures such as a norbornane structure, an adamantane structure, a tricyclodecane structure, and a tetracyclododecane structure.

When at least one of R¹⁴ and R¹⁵ is a monovalent unsaturated hydrocarbon group, examples of the monovalent unsaturated hydrocarbon group include a C1 to C20 monovalent unsaturated chain hydrocarbon group, a C3 to C20 monovalent alicyclic unsaturated hydrocarbon group, and a C6 to C20 monovalent aromatic hydrocarbon group. Specific examples thereof include the same groups as exemplified in relation to the aforementioned hydrocarbon groups.

Examples of the C1 to C20 monovalent oxyhydrocarbon group represented by R¹⁷, R¹⁸, R¹⁹, R³², R³³, or R³⁴ include groups formed by incorporating an oxygen atom into the chemical bond side end of any of C1 to C20 monovalent hydrocarbon groups represented by the aforementioned R¹³ or the like, as exemplified above. The monovalent oxyhydrocarbon group represented by R¹⁷ or the like is preferably, among others, an alkoxy group, a cycloalkoxy group, or a cycloalkylalkoxy group.

When the group represented by R¹³, R¹⁴, R¹⁵, R¹⁷, R¹⁸, R¹⁹, R³², R³³, or R³⁴ has a substituent, examples of the substituent include a halogen atom, a hydroxy group, and a C1 to C3 alkoxy group.

Examples of the monovalent substituent represented by R³⁵ or R³⁶ include a C1 to C3 alkyl group, a C1 to C3 alkoxy group, a hydroxy group, and a halogen atom. Each of the parameters g1 and g2 is preferably 0 to 2, more preferably 0 or 1.

Specific examples of the structural unit (I-1) include structural units represented by the following formulas.

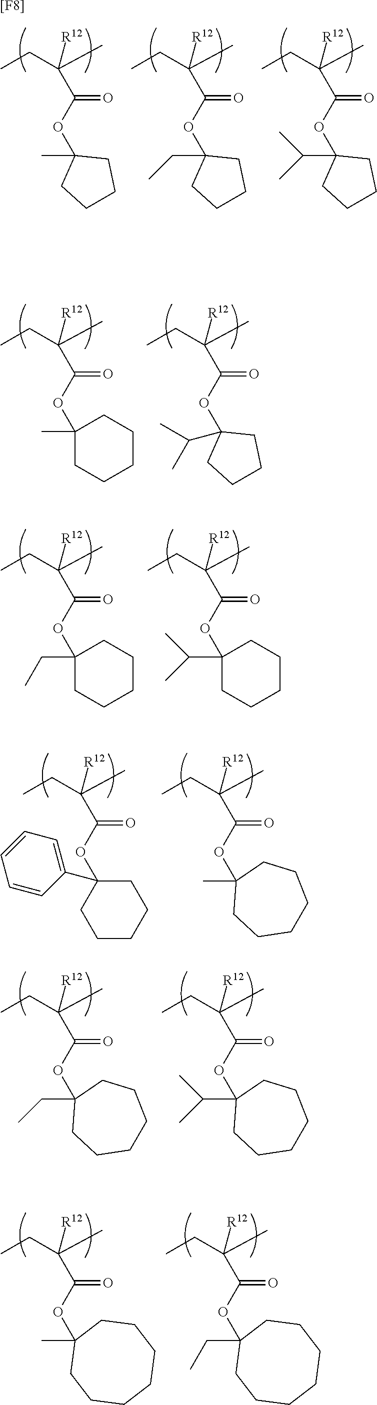

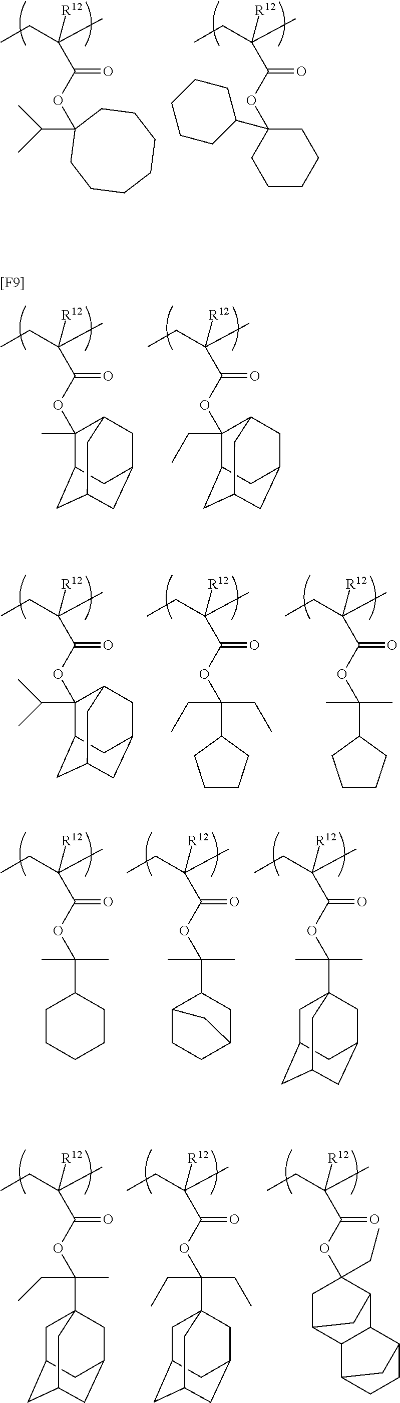

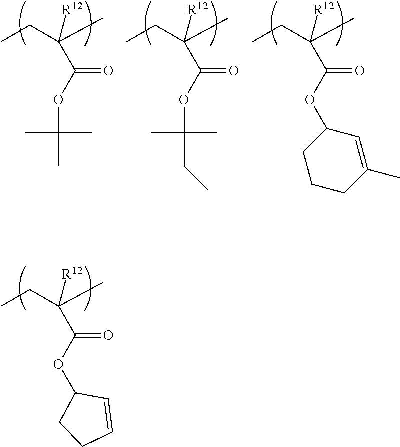

(In the formulas, R¹² represents a hydrogen atom, a fluorine atom, a methyl group, or a trifluoromethyl group.)

Specific examples of the structural unit (1-2) include structural units represented by the following formulas.

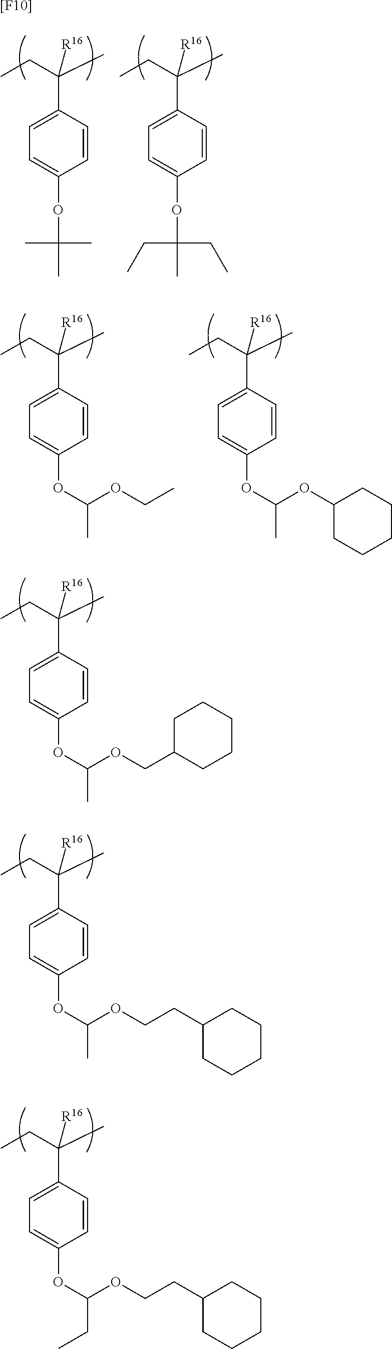

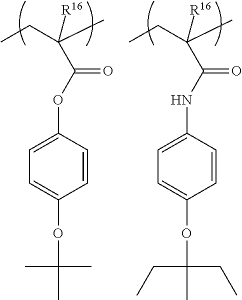

(In the formulas, R¹⁶ represents a hydrogen atom, a fluorine atom, a methyl group, or a trifluoromethyl group.)

Specific examples of the structural unit (1-3) include structural units represented by the following formulas.

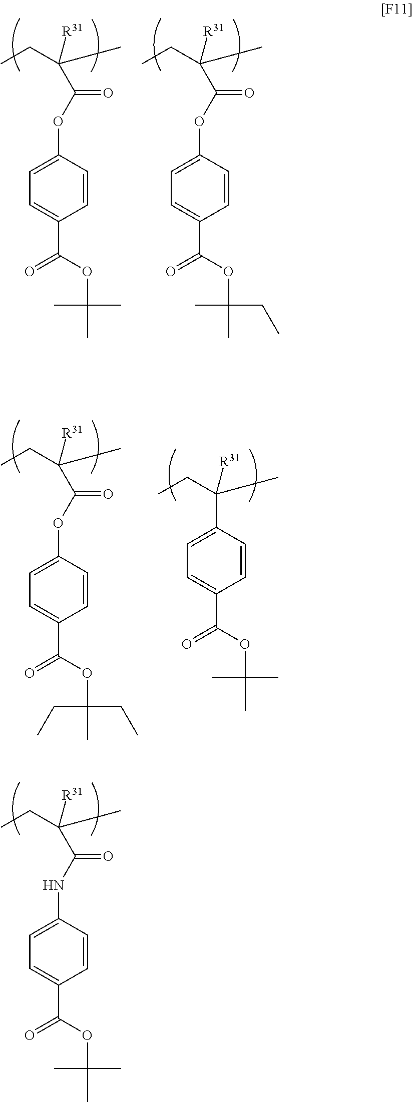

(In the formulas, R³¹ represents a hydrogen atom, a fluorine atom, a methyl group, or a trifluoromethyl group.)

In the polymer (A), the relative amount of structural unit (I) in all the structural units forming the polymer (A) is preferably 20 mols or more, more preferably 25 mol % or more, still more preferably 30 mol % or more. Also, the relative amount of structural unit (I) in all the structural units forming the polymer (A) is preferably 80 mol % or less, more preferably 75 mol % or less, still more preferably 70 mol % or less. Adjusting the structural unit (I) content to satisfy the aforementioned conditions is preferred, since considerable difference in dissolution rate with respect to a developer between the light-exposed part and the light-unexposed part can be sufficiently attained, whereby a favorable shape of the resist film pattern can be provided.

In addition to the structural unit (I), the polymer (A) may further include a structural unit differing from the structural unit (I) (hereinafter may also be referred to as an “additional structural unit”). Examples of the additional structural unit include the following structural units (II) to (V).

### Structural Unit (II)

The polymer (A) may further include a structural unit having a hydroxy group bonded to an aromatic ring (hereinafter may also be referred to as a “structural unit (II)”). Incorporation of a hydroxy group bonded to an aromatic ring into the polymer (A) is preferred, since lithographic characteristics of the first composition (e.g., LWR performance and CDU (critical dimension uniformity) performance) can be further improved, and dissolution of the light-unexposed part in a developer can be highly suppressed, whereby development failure can be satisfactorily reduced.

In the structural unit (II), examples of the aromatic ring to which a hydroxy group is bound include a benzene ring, a naphthalene ring, an anthracene ring, and a phenanthrene ring. Of these, a benzene ring and a naphthalene ring are preferred, with a benzene ring being more preferred. No particular limitation is imposed on the number and position of the hydroxy group(s) bound to the aromatic ring in the structural unit (II). The number of the hydroxy group(s) bound to the aromatic ring is preferably 1 to 3, more preferably 1 or 2. Examples of the structural unit (II) include the structural units represented by the following formula (ii).

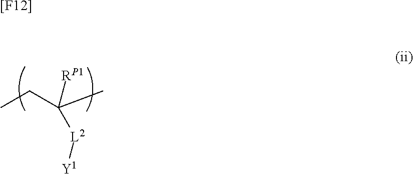

(In the formula (ii), R^{P1} represents a hydrogen atom, a fluorine atom, a methyl group, or a trifluoromethyl group; L² represents a single bond, —O—, —CO—, —COO—, or —CONH—; and Y¹ represents a monovalent group having a hydroxy group bonded to an aromatic ring.)

In the above formula (ii), R^{P1} is preferably a hydrogen atom or a methyl group, and L² is preferably a single bond or —COO—, from the viewpoint of co-polymerizability of a monomer to form the structural unit (II).

Specific examples of the structural unit (II) include the structural units represented by the following formulas.

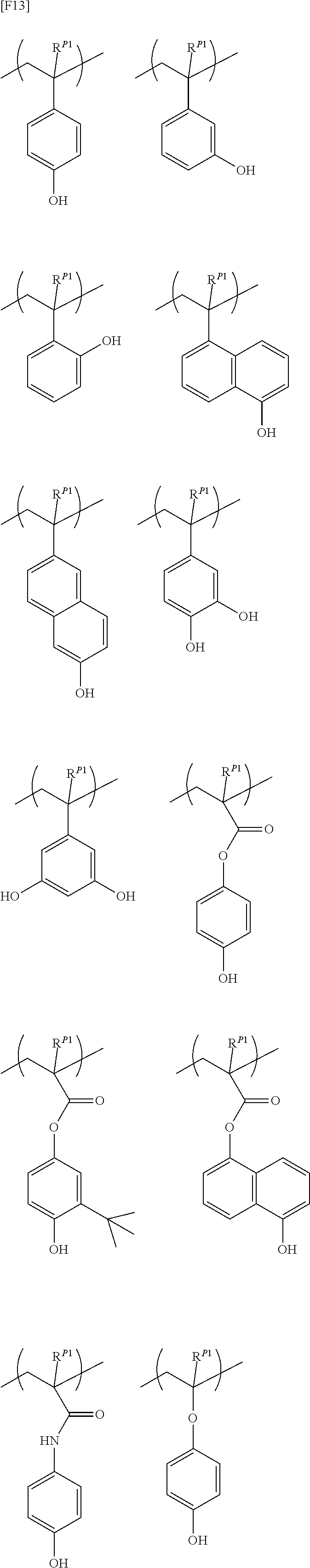

(In the formulas, R^{P1} represents a hydrogen atom, a fluorine atom, a methyl group, or a trifluoromethyl group.)

When the polymer (A) includes the structural unit (II), in the polymer (A), the relative amount of structural unit (II) in all the structural units forming the polymer (A) is preferably 5 mol % or more, more preferably 10 mol % or more, still more preferably 15 mol % or more. Also, the relative amount of structural unit (II) in all the structural units forming the polymer (A) is preferably 90 mol % or less, more preferably 80 mol % or less, still more preferably 60 mol % or less. By adjusting the structural unit (II) content to satisfy the above conditions, lithographic characteristics of the first composition can be further enhanced, which is preferred.

Notably, the polymer (A) may include a structural unit having a partial structure in which a hydroxy group and an acid-releasable group are bound to the same aromatic ring. As used herein, a structural unit having a partial structure in which a hydroxy group and an acid-releasable group are bound to the same aromatic ring is categorized in the structural unit (I).

### Structural Unit (III)

The polymer (A) may further includes a structural unit having a ring structure formed from a lactone structure, a cyclic carbonate structure, a sultone structure, or a combination of two or more members thereof (hereinafter may also be referred to as a “structural unit (III)”). Incorporation of the structural unit (III) into the polymer (A) is preferred, since solubility of the composition in a developer can be controlled, whereby lithographic characteristics of the first composition can be further enhanced. Also, the presence of the structural unit (III) in the polymer (A) can improve close adhesion between a substrate and a resist film formed from the first composition.

Examples of the structural unit (III) includes the structural units represented by the following formulas.

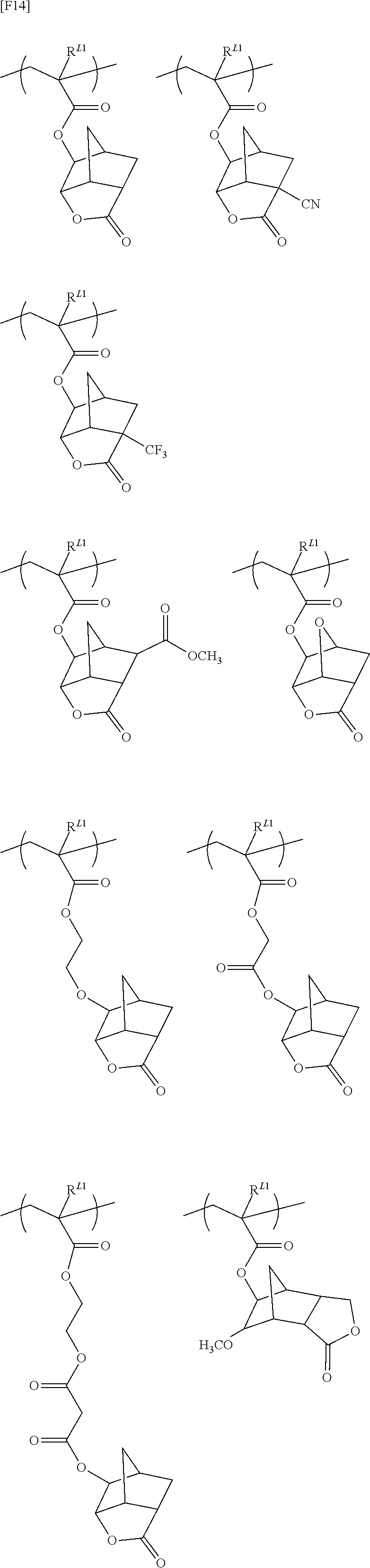

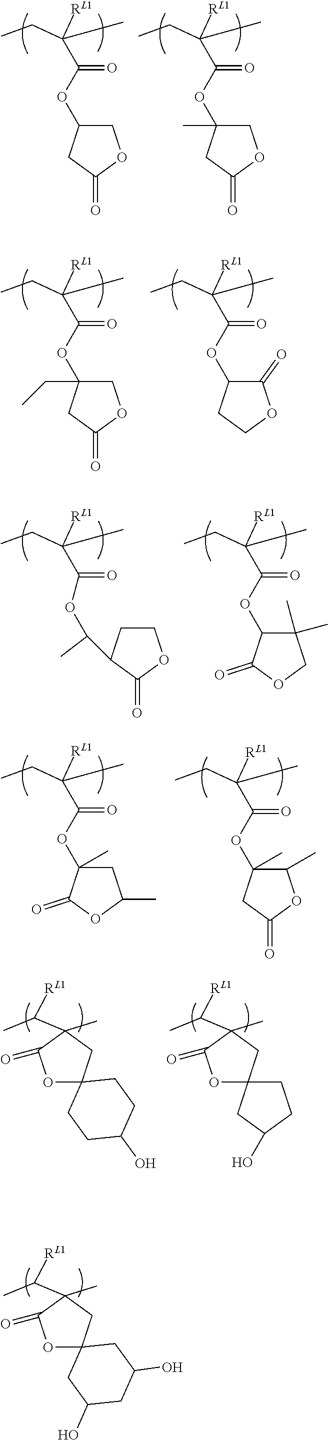

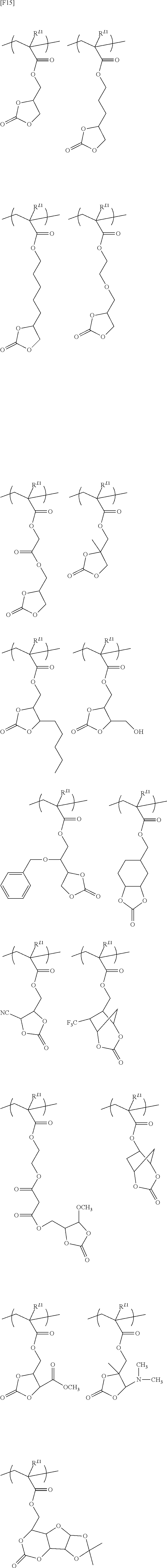

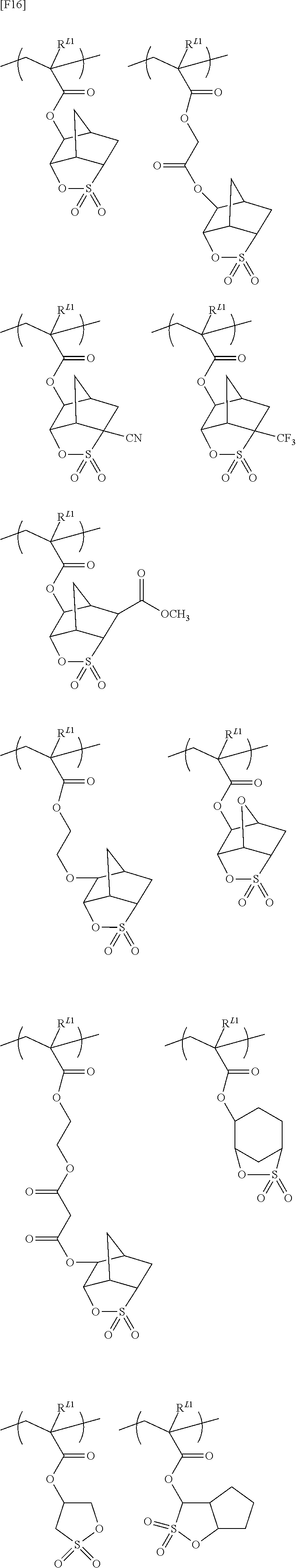

(In the formulas, Ru represents a hydrogen atom, a fluorine atom, a methyl group, or a trifluoromethyl group.)

When the polymer (A) includes the structural unit (III), the relative amount of structural unit (III) in all the structural units forming the polymer (A) is preferably 1 mol % or more, more preferably 3 mol % or more, still more preferably 5 mol % or more. Also, the relative amount of structural unit (III) in all the structural units forming the polymer (A) is preferably 50 mol % or less, more preferably 30 mol % or less, still more preferably 15 mol % or less. Adjusting of the structural unit (III) content to satisfy the above conditions is preferred, since the lithographic characteristics of the first composition can be enhanced, and close adhesion between the resist film obtained from the first composition and a substrate can be enhanced.

### Structural Unit (IV)

The polymer (A) may further include a structural unit having an alcoholic hydroxy group (except for a structural unit corresponding to the structural units (I) to (III)) (hereinafter may also be referred to as a “structural unit (IV)”). As used herein, the term “alcoholic hydroxy group” refers to a group having a structure in which a hydroxy group is directly bound to an aliphatic hydrocarbon group. The aliphatic hydrocarbon group may be a chain hydrocarbon group or an alicyclic hydrocarbon group. By incorporating the structural unit (IV) into the polymer (A), solubility of the polymer (A) in a developer can be improved, whereby lithographic characteristics of the first composition can be further improved, which is preferred.

The structural unit (IV) is preferably a structural unit derived from a monomer having an alcoholic hydroxy group and a group involved in polymerization. Examples of the structural unit (IV) include the structural units represented by the following formulas.

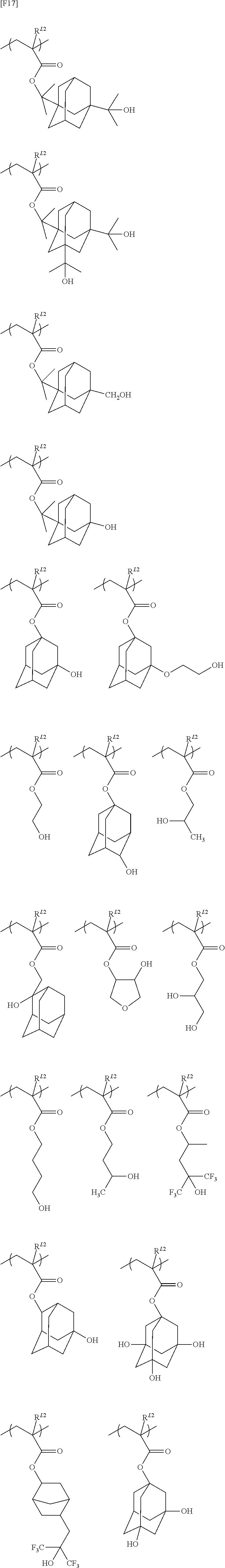

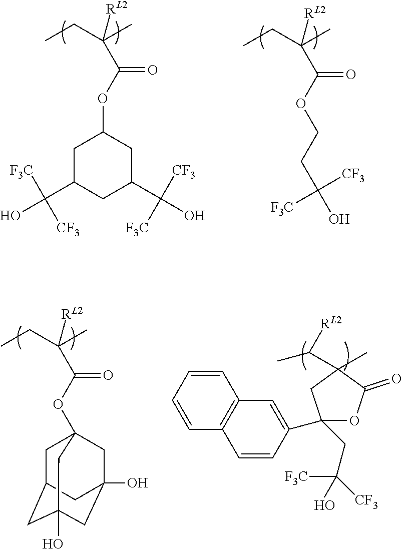

(In the formulas, R^{L2} represents a hydrogen atom, a fluorine atom, a methyl group, or a trifluoromethyl group.)

When the polymer (A) includes the structural unit (IV), the relative amount of structural unit (IV) in all the structural units forming the polymer (A) is preferably 1 mol % or more, more preferably 3 mol % or more. Also, the relative amount of structural unit (IV) in all the structural units forming the polymer (A) is preferably 30 mol % or less, more preferably 20 mol % or less.

### Structural Unit (V)

The polymer (A) may further include a structural unit having an iodo group (hereinafter may also be referred to as a “structural unit (V)”). Through incorporation of an iodo group into the polymer (A), lithographic characteristics (e.g., LWR performance and CDU performance) of the first composition can be further enhanced, and a wider process window can be secured. From the viewpoint of achieving high effects of improving lithographic characteristics and process window, the polymer (A) preferably has an iodo group bound to an aromatic ring (preferably, a benzene ring). The number of iodo group(s) bound to a single aromatic ring is, for example, 1 to 5.

Examples of the structural unit (V) include a structural unit including a radiation-sensitive onium cation and an organic anion and has an iodo group, a structural unit derived from 4-iodostyrene, and a structural unit derived from 3-iodostyrene. Preferably, the structural unit (V) is a structural unit derived from an onium salt compound having a group involved in polymerization. Through incorporation of such a structural unit into the polymer (A), the effect of reducing development residue, and lithographic characteristics can be enhanced. The iodo group may belong to a radiation-sensitive onium cation moiety or to an organic anion moiety, or to both moieties.

Specific examples of the structural unit (V) include structural units represented by the following formula (va-1), those represented by the following formula (va-2), those represented by the following formula (va-3), and a structural unit having a partial structure in which L⁻ in the aforementioned formula (2) is —COO⁻.

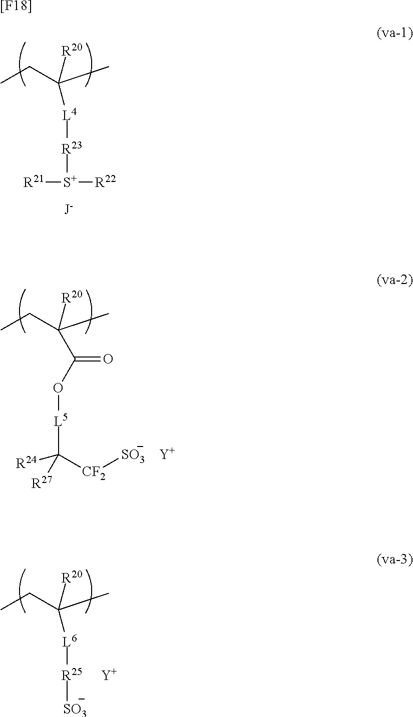

(In the formula (va-1), R²⁰ represents a hydrogen atom, a fluorine atom, a methyl group, or a trifluoromethyl group; L⁴ represents a single bond, —O—, or —COO−. R²³ represents a C1 to C6 substituted or unsubstituted alkanediyl group, a C2 to C6 substituted or unsubstituted alkenediyl group, or a C6 to C12 substituted or unsubstituted arylene group; each of R²¹ and R²² independently represents a C1 to C12 substituted or unsubstituted alkyl group, a C2 to C12 substituted or unsubstituted alkenyl group, or a C6 to C20 substituted or unsubstituted aryl group; J-represents a monovalent anion; and one or more members of L⁴, R²¹, R²², R²³, and J⁻ in formula (va-1) have an iodo group;
in the formula (va-2), R²⁰ represents a hydrogen atom, a fluorine atom, a methyl group, or a trifluoromethyl group; L⁵ represents a single bond, **—R^{30a}—CO—O—, **—R^{30a}—O—, or **—R^{30a}—O—CO— (wherein “**” represents a chemical bond with oxygen); R^{30a} represents a C1 to C12 substituted or unsubstituted alkanediyl group, a C6 to C20 substituted or unsubstituted arylene group, a C6 to C12 substituted or unsubstituted aralkylene group, a divalent group formed by inserting —O—, —CO—, or —COO— into a carbon-carbon bond of a C2 to C12 substituted or unsubstituted alkanediyl group, or a divalent group formed by inserting —O—, —CO—, or —COO⁻ into a carbon-carbon bond of a C6 to C12 substituted or unsubstituted aralkylene group; each of R²⁴ and R²⁷ independently represents a hydrogen atom, a fluorine atom, a C1 to C10 alkyl group, or a C1 to C10 fluoroalkyl group; Y⁺ represents a monovalent cation; and one or more members of L⁵ and Y⁺ in formula (va-2) have an iodo group; and
in the formula (va-3), R²⁰ represents a hydrogen atom, a fluorine atom, a methyl group, or a trifluoromethyl group; L⁶ represents a single bond, a C1 to C6 substituted or unsubstituted alkanediyl group, a C2 to C6 substituted or unsubstituted alkenediyl group, a C6 to C12 substituted or unsubstituted arylene group, a C6 to C12 substituted or unsubstituted aralkylene group, **—CO—O—R^{30b}—, or **—CO—NH—R^{30b}. (wherein “**” represents a chemical bond with a carbon atom to which R²⁰ is bound); R^{30b} represents a C1 to C6 substituted or unsubstituted alkanediyl group, a C6 to C12 substituted or unsubstituted aralkylene group, a divalent group formed by inserting —O—, —CO—, or —COO− into a carbon-carbon bond of a C2 to C12 substituted or unsubstituted alkanediyl group, or a divalent group formed by inserting —O—, —CO—, or —COO− into a carbon-carbon bond of a C6 to C12 substituted or unsubstituted aralkylene group; R²⁵ represents a single bond, ***—CO—O—R²⁶—, ***—O—CO—R²⁶—, ***—NH—CO—R²⁶—, ***—CO—NH—R²⁶, or ***—O—NH—R²⁶— (wherein “***” represents a chemical bond with L⁶); R²⁶ represents a C1 to C6 substituted or unsubstituted alkanediyl group, a C2 to C6 substituted or unsubstituted alkenediyl group, or a C6 to C12 substituted or unsubstituted arylene group; Y⁺ represents a monovalent cation; and one or more members of L⁶, R²⁵, and Y⁺ in formula (va-3) have an iodo group.)

In the formulas (va-1) to (va-3), when each of the groups R²¹ and R²² is a substituted alkyl group, a substituted alkenyl group, or a substituted aryl group, and each of the groups R²³, R^{30a}, R^{30b}, L⁶, and R²⁶ is a substituted alkanediyl group, a substituted alkenediyl group, or a substituted arylene group, examples of the substituent include a fluorine atom, a chlorine atom, a bromine atom, an iodine atom, an alkoxy group, a cycloalkyloxy group, an ester group, an alkylsulfonyl group, a cycloalkylsuofonyl group, a hydroxy group, a carboxy group, a cyano group, a nitro group, an acetyl group, and a fluoroacetyl group. The structural unit represented by the aforementioned formula (va-3) may have a partial structure represented by the aforementioned formula (2b).

The cation (Y⁺) in the formulas (va-2) and (va-3) preferably has a triarylsulfonium cation structure or a diaryliodoniumcation structure. From the viewpoint of enhancing sensitivity of the first composition, a fluoro group or a fluoroalkyl group is preferably bound to an aromatic ring in the triarylsulfonium cation structure or the diaryliodonium cation structure (i.e., an aromatic ring bound to S⁺ or I⁺). The fluoroalkyl group is preferably a trifluoromethyl group.

Specific examples of the structural unit (V) include the structural units represented by the following formulas (v-1) to (v-15).

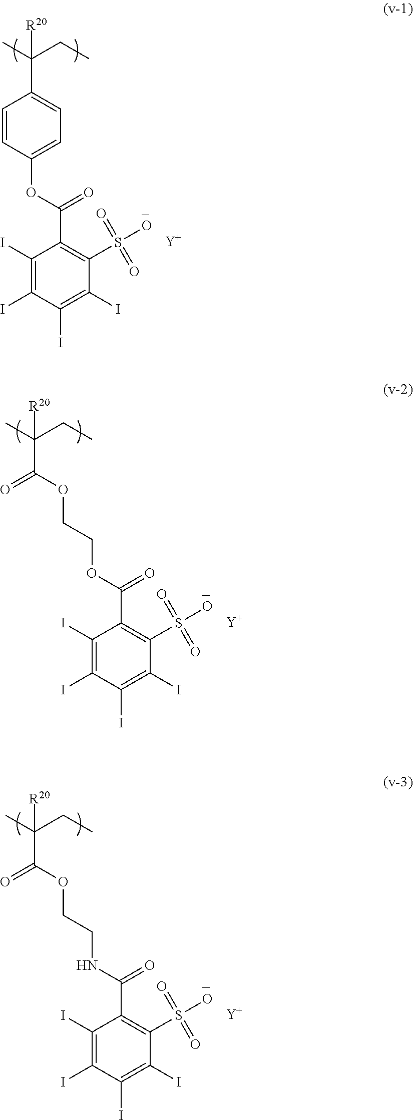

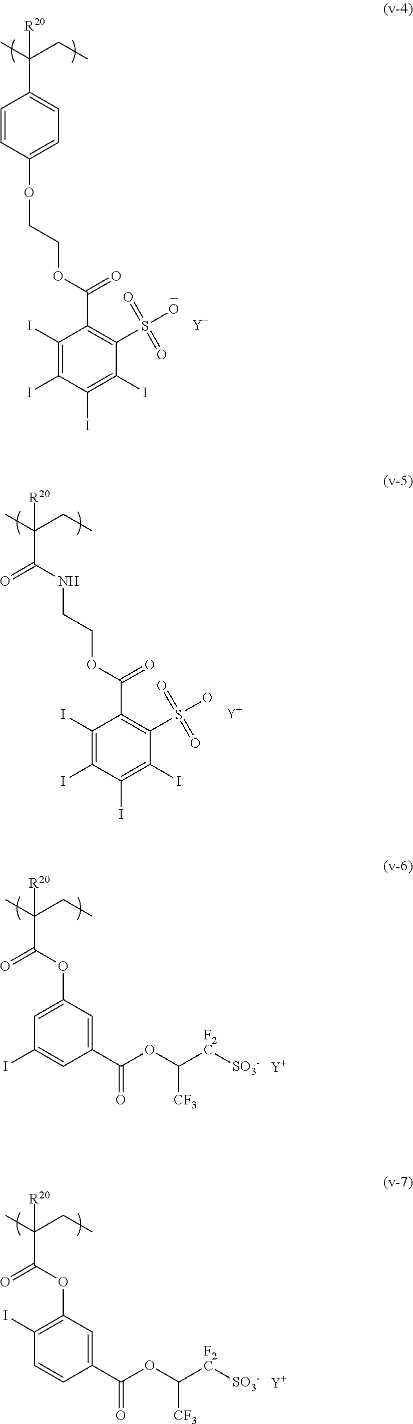

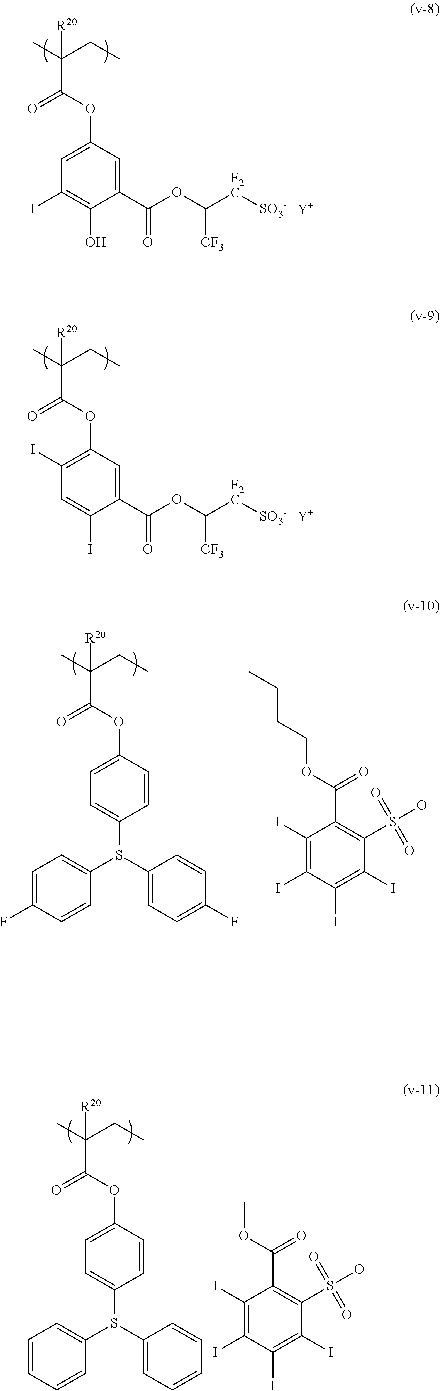

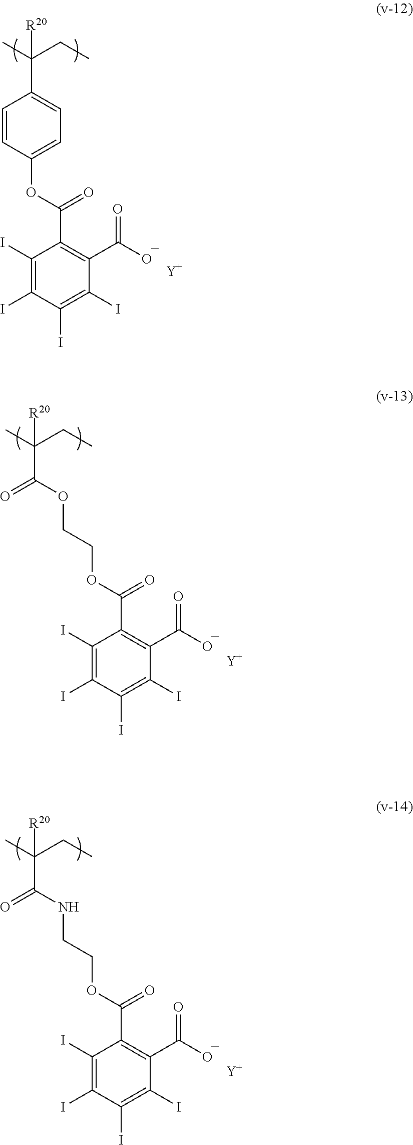

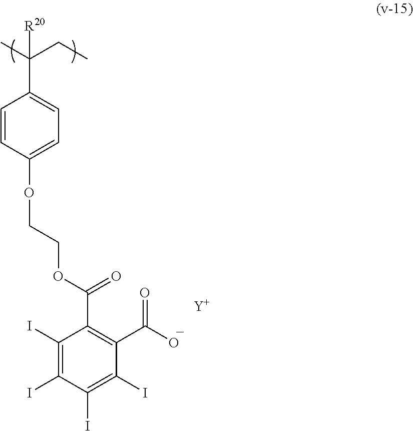

(In the formulas, R²⁰ is a hydrogen atom, a fluorine atom, a methyl group, or a trifluoromethyl group; and Y⁺ represents a monovalent cation.)

Notably, the polymer including a structural unit having a partial structure represented by the aforementioned formula (2b) (e.g., a structural unit represented by any of the aforementioned formulas (v-1) to (v-5)) also corresponds to a polymer (PB). In other words, the polymer (A) incorporated into the first composition may be a polymer (PB) having an acid-releasable group and a partial structure represented by the aforementioned formula (2b). Also, the polymer (A) incorporated into the first composition may be a polymer having an acid-releasable group and a partial structure in which L⁻ in the aforementioned formula (2) is —COO⁻.

When the polymer (A) includes the structural unit (V), the relative amount of structural unit (V) in all the structural units forming the polymer (A) is preferably 1 mol % or more, more preferably 3 mol % or more, still more preferably 5 mol % or more. Also, the relative amount of structural unit (V) in all the structural units forming the polymer (A) is preferably 50 mol % or less, more preferably 40 mol % or less, still more preferably 30 mol % or less. By adjusting the structural unit (V) content to satisfy the aforementioned conditions, a drop in resolution possibly due to diffusion of acid can be suppressed, to thereby enhance lithographic performance of the first composition.

In addition the aforementioned structural units, examples of the additional structural unit include a structural unit having a cyano group, a nitro group, or a sulfonamide group (e.g., a structural unit derived from 2-cyanomethyladamantan-2-yl (meth)acrylate); a structural unit containing a fluorine atom and differing from the aforementioned structural units (I) to (V) (e.g., a structural unit derived from 2,2,2-trifluoroethyl (meth)acrylate or a structural unit derived from 1,1,1,3,3,3-hexafluoropropan-2-yl (meth)acrylate); a structural unit having a non-acid-releasable hydrocarbon group (e.g., a structural unit derived from styrene, a structural unit derived from vinylnaphthalene, or a structural unit derived from n-pentyl (meth)acrylate); and a structural unit including a radiation-sensitive onium cation and an organic anion and differing from the aforementioned structural unit (V). The relative amount of any of these structural units may be appropriately set in accordance with the type of each structural unit, so long as the effects of the present disclosure are not impaired.

In the first composition, the polymer (A) content, with respect to the entire amount of the solid contained in the first composition, is preferably 50 mass % or more, more preferably 55 mass % or more, still more preferably 60 mass or more. Also, the polymer (A) content, with respect to the entire amount of the solid contained in the first composition, is preferably 99 mass % or less, more preferably 98 mass % or less, still more preferably 95 mass % or less. The polymer (A) generally forms a base resin of the first composition. The first composition may contain the polymer (A) singly or in combination of two or more species. As used herein, the term “base resin” refers to a polymer component accounting for 50 mass or more of the entire amount of the solid contained in the present composition. The “solid content” refers to the components contained in the present composition other than solvent.

The polymer (A) can be synthesized through, for example, polymerizing monomers for providing corresponding structural units in the presence of a known radical polymerization initiator or the like in an appropriate solvent.

The weight average molecular weight (Mw) of the polymer (A), which is determined through gel permeation chromatography (GPC) and is reduced to polystyrene, is preferably 1,000 or more, more preferably 2,000 or more, still more preferably 3,000 or more, yet more preferably 4,000 or more. Also, the Mw of the polymer (A) is preferably 50,000 or less, more preferably 30,000 or less, still more preferably 20,000 or less, yet more preferably 15,000 or less. Adjusting the Mw of the polymer (A) so as to satisfy the above conditions is preferred, since coatability of the first composition can be improved, and development failure can be sufficiently suppressed.

The ratio (Mw/Mn) of Mw to the number average molecular weight (Mn) of the polymer (A), which is determined through gel permeation chromatography (GPC) and is reduced to polystyrene, is preferably 5.0 or less, more preferably 3.0 or less, still more preferably 2.0 or less, yet more preferably 1.8 or less. Also, the Mw/Mn of the polymer (A) is generally 1.0 or more, and may be 1.1 or more.

### [Compound (CB)]

The compound (CB) is a radiation-sensitive acid-generator that can generate an acid through light exposure. As represented by the aforementioned formula (1b), the compound (CB) is formed of an organic anion and a cation. Through action of radiation, the compound (CB) releases an organic anion via decomposition of the radiation-sensitive cation, and the thus-released organic anion bonds to hydrogen extracted from a component contained in the present composition (e.g., the radiation-sensitive acid-generator itself or a solvent), whereby an acid derived from the organic anion is generated. As used herein, the term “radiation” encompasses to an electron beam (e.g., visible light, UV ray, far-UV ray, or extreme ultraviolet ray (EUV)) and an electromagnetic wave (e.g., X-ray or γ-ray).

Preferably, the compound (CB) is incorporated into the first composition as a radiation-sensitive acid-generating agent. More specifically, preferably, the first composition contains the polymer (A) and the compound (CB) serving as the radiation-sensitive acid-generating agent. Under such conditions, an acid-releasable group is released by the action of an acid generated upon irradiation of the compound (CB) with radiation, to thereby form an acidic group (a sulfonic acid group in the case of the compound (CB)), whereby solubility of the polymer (A) in a developer is modified.

### (Organic Anion)

In the aforementioned formula (1b), when A¹ is —NR³—, examples of the monovalent organic group represented by R³ include a C1 to C12 hydrocarbon group. Specific examples of the C1 to C12 hydrocarbon group include a C1 to C12 monovalent chain hydrocarbon group, a C3 to C12 monovalent alicyclic hydrocarbon group, and a C6 to C12 monovalent aromatic hydrocarbon group. Further specific examples thereof include groups exemplified as the C1 to C20 monovalent hydrocarbon group represented by R¹³ or the like in the aforementioned formulas (i-1) to (i-3) and having a corresponding number of carbon atoms.

From the viewpoints of ease of synthesis of the compound (CB) and availability of starting materials, A¹ is preferably —O— or —S—, more preferably —O—.

Examples of the monovalent organic group represented by R¹ include a C1 to C20 substituted or unsubstituted monovalent hydrocarbon group, and a C2 to C20 monovalent group formed by inserting-O—, —CO—, —COO—, NH—, —CONH—, —S—, etc. into a carbon-carbon bond of the substituted or unsubstituted hydrocarbon group.

Specific examples of the case where R¹ is a C1 to C20 monovalent hydrocarbon group include a C1 to C20 monovalent chain hydrocarbon group, a C3 to C20 monovalent alicyclic hydrocarbon group, and a C6 to C20 monovalent aromatic hydrocarbon group. Further specific examples thereof include the same groups as exemplified in relation to the C1 to C20 monovalent hydrocarbon group represented by R¹³ or the like in the aforementioned formulas (i-1) to (i-3).

When R¹ is a substituted C1 to C20 monovalent hydrocarbon group, examples of the substituent include a fluorine atom, a chlorine atom, a bromine atom, an iodine atom, a hydroxy group, a carboxy group, a cyano group, a nitro group, an aldehyde group, an acetyl group, an amino group, a sulfanyl group, a cyclic ether group, a cyclic acetal group, a furyl group, a (meth)acryloyloxy group, a (meth)acryloylamino group, a vinyl ether group, and a vinyl phenyl ether group. No particular limitation is imposed on the number of substituent(s), and the number is, for example, 1 to 5. Notably, one or more substituents may belong to R¹.

From the viewpoint of further enhancing the sensitivity of the first composition, R¹ preferably has a polar group. Examples of preferred polar groups include a hydroxy group, a carboxy group, a cyano group, a nitro group, an aldehyde group, an acetyl group, a furyl group, and a cyclic acetal group.

R¹ is preferably, among the aforementioned examples, a C1 to C20 monovalent organic group, more preferably a C1 to C20 substituted or unsubstituted monovalent hydrocarbon group, or a C2 to C20 monovalent group formed by inserting —O—, —CO—, —COO—, —NH—, —CONH—, or —S— into a carbon-carbon bond of the substituted or unsubstituted hydrocarbon group.

When R^{2b} is a monovalent hydrocarbon group, specific examples thereof include the same groups as exemplified in relation to the case where R³ is a C1 to C12 monovalent hydrocarbon group. When R^{2b} is a substituted monovalent hydrocarbon group, examples of the substituent replacing a hydrogen atom of the hydrocarbon group include the same substituents as exemplified in relation to R¹. When n is ≥2, and two Ribs in formula (1b) are combined to form a ring structure with a carbon atom to which Ribs are bound, the ring structure and the benzene ring in the formula (1b) preferably form a condensed ring structure. The condensed ring structure is preferably a ring structure exhibiting aromaticity, more preferably a naphthalene ring structure formed with the benzene ring in the formula (1b). From the viewpoint of increasing the amount of X¹ incorporated so as to further enhance the sensitivity of the first composition, the parameter n is preferably 0 to 2, more preferably 0 or 1, still more preferably 0.

The compound (CB) has a partial structure in which at least one member selected from the group consisting of a chlorine atom, a bromine atom, and an iodine atom (hereinafter may also be referred to as a “particular atom”) is bound to a benzene ring. The particular atom (i.e., X¹) is preferably an iodine atom, from the viewpoint of achieving higher sensitivity of the first composition. From the viewpoint of enhancing the sensitivity of the first composition, the number of the particular atoms bound to a benzene ring (i.e., m) is preferably ≥2, more preferably ≥3, still more preferably 4. When m is ≥2, the species of the particular atoms in a plurality of X¹s may be identical to or different from one another. When m is ≥2, one or more species of the particular atoms in a plurality of X¹s are preferably an iodine atom. More preferably, all the particular atoms are an iodine atom. No particular limitation is imposed on the bonding position of X¹ with respect to the benzene ring in the aforementioned formula (1b). In other words, the bonding position of X¹ may be any of o-, m-, and p-, with respect to group “—SO₃⁻”.

From the viewpoint of sensitivity of the first composition, in the compound (CB), one or more X¹s present in the aforementioned formula (1b) are preferably an iodine atom. More specifically, in a preferred mode, m in the aforementioned formula (1b) is 1, and X¹ is an iodine atom; and m is ≥2, and one or more members of a plurality of X¹s is an iodine atom. Particularly, in a preferred mode of the compound (CB), m in the aforementioned formula (1b) is ≥2, and one or more members of a plurality of X¹s are an iodine atom. In a more preferred mode, m is ≥2, and two or more X¹s are an iodine atom. In a still preferred mode, m is ≥3, and three or more members of X¹s are an iodine atom. In a particularly preferred mode, m is 4, and all the four X¹s are an iodine atom, from the viewpoint of the sensitivity of the first composition.

### (Cation)

M⁺ in the aforementioned formula (1b) is preferably an organic cation, more preferably a radiation-sensitive onium cation.

No particular limitation is imposed on the structures of M⁺. From the viewpoint of achieving favorable lithographic characteristics of the first composition, M⁺ preferably has a sulfonium cation structure, an iodonium cation structure, or an ammonium cation structure, more preferably a sulfonium cation structure or an iodonium cation structure, still more preferably a triarylsulfonium cation structure or a diaryliodonium cation structure. Also, from the viewpoint of achieving higher sensitivity of the first composition, M⁺ preferably has a triarylsulfonium cation structure or a diaryliodonium cation structure in which a fluoro group or a fluoroalkyl group (preferably, a fluoromethyl group) is bound to an aromatic ring (i.e., an aromatic ring bound to S⁺ or I⁺).

Specific examples of M⁺ include a cation represented by the following formula (4), a cation represented by the following formula (5), a cation represented by the following formula (6), and a cation represented by the following formula (7). Notably, the cation represented by the following formula (4) corresponds to a cation having a triarylsulfonium cation structure, and the cation represented by the following formula (5) corresponds to a cation having a diaryliodonium cation structure.

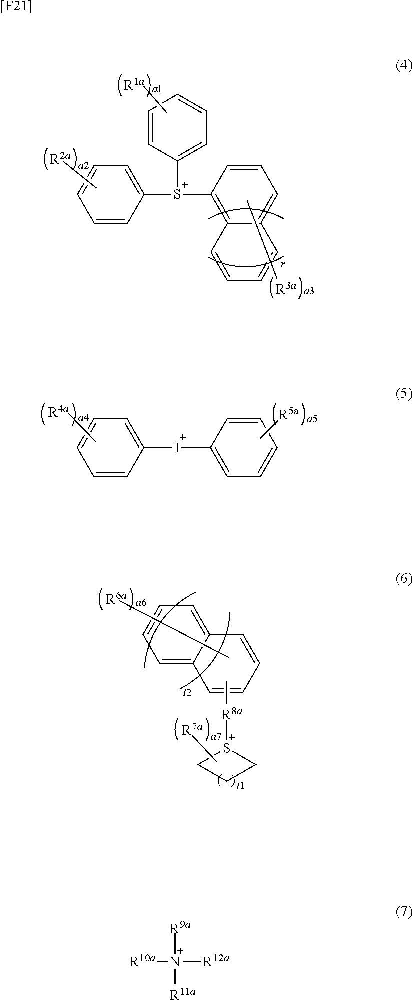

(In the formula (4), each of R^{1a} and R^{2a} independently represents a monovalent substituent, or a single bond or a divalent group linked to a ring formed by combining R^{1a} and R^{2a}; R^{3a} represents a monovalent substituent; each of a1 and a2 is an integer of 0 to 5; a3 is an integer of 0 to (2×r+5); and r is 0 or 1);
in the formula (5), each of R^{4a} and R^{5a} independently represents a monovalent substituent; and each of a4 and a5 is an integer of 0 to 5;
in the formula (6), a6 is an integer of 0 to 7; when a6 is 1, R^{6a} is a C1 to C20 monovalent organic group, a hydroxy group, a nitro group, or a halogen atom; when a6 is 2 or greater, a plurality of R^{6a}s, which are identical to or different from one another, represent a C1 to C20 monovalent organic group, a hydroxy group, a nitro group, or a halogen atom, or a 4- to 20-membered ring structure formed with a carbon atom to which a linked moiety formed of two or more of the plurality of R^{6a}s are bonded; a7 is an integer of 0 to 6; when a7 is 1, R^{7a} is a C1 to C20 monovalent organic group, a hydroxy group, a nitro group, or a halogen group; when a7 is 2 or greater, a plurality of Ras, which are identical to or different from one another, represent a C1 to C20 monovalent organic group, a hydroxy group, a nitro group, or a halogen atom, or a 3- to 20-membered ring structure formed with a carbon atom to which a linked moiety formed of two or more of the plurality of R^{7a}s are bonded; t1 is an integer of 0 to 3; R^{8a} represents a single bond or a C1 to C20 divalent organic group; and t2 is 0 or 1; and
in the formula (7), each of R^{9a} and R^{10a} independently represents a hydrogen atom or a monovalent organic group, or a ring structure formed with a nitrogen atom to which a linked moiety formed of R^{9a} and R^{10a} is bonded; and each of R^{11a} and R^{12a} independently represents a hydrogen atom or a monovalent organic group, or a ring structure formed with a nitrogen atom to which a linked moiety formed of R^{11a} and R^{12a} is bonded.)

In the aforementioned formulas (4) and (5), examples of the monovalent substituent represented by any of R^{1a}, R^{2a}, R^{3a}, R^{4a}, and R^{5a} (hereinafter denoted by “R^{1a} to R^{5a}”) include a fluorine atom, a chlorine atom, a bromine atom, an iodine atom, a substituted or unsubstituted alkyl group, a substituted or unsubstituted alkoxy group, a substituted or unsubstituted cycloalkyl group, a substituted or unsubstituted cycloalkyloxy group, an ester group, an alkylsulfonyl group, a cycloalkylsulfonyl group, a hydroxy group, a carboxy group, a cyano group, and a nitro group.

The alkyl group represented by any of R^{1a} to R^{5a} may be linear-chain or branched. The alkyl group is preferably a C1 to C10, and examples thereof include methyl, ethyl, n-propyl, i-propyl, n-butyl, i-butyl, sec-butyl, t-butyl, n-pentyl, and neopentyl. Of these, the alkyl group represented by any of R^{1a} to R^{5a} is preferably a C1 to C5 alkyl group, with methyl, ethyl, n-butyl, and t-butyl being more preferred.

When any of R^{1a} to R^{5a} is a substituted or unsubstituted alkoxy group, specific examples thereof include a group in which any of the above-exemplified substituted or unsubstituted alkyl groups is attached to the alkyl moiety forming the alkoxy group. The alkoxy group is particularly preferably methoxy, ethoxy, n-propoxy, or n-butoxy.

The cycloalkyl group represented by any of R^{1a} to R^{5a} may be monocyclic or polycyclic. Examples of the monocyclic cycloalkyl group include cyclopropyl, cyclobutyl, cyclopentyl, cyclohexyl, and cyclooctyl. Examples of the polycyclic cycloalkyl group include norbornyl, adamantyl, tricyclodecyl, and tetracyclododecyl.

When any of R^{1a} to R^{5a} is a substituted or unsubstituted cycloalkyloxy group, specific examples thereof include a group in which any of the above-exemplified substituted or unsubstituted cycloalkyl groups is bound to the cycloalkyl moiety forming the cycloalkyloxy group. The cycloalkyloxy group represented by any of R^{1a} to R^{5a} is preferably a cyclopentyloxy group or a cyclohexyloxy group.

When the any of R^{1a} to R^{5a} is a substituted alkyl group, a substituted alkoxy group, a substituted cycloalkyl group, or a substituted cycloalkyloxy group, examples of the substituent include a fluorine atom, a chlorine atom, a bromine atom, an iodine atom, a hydroxy group, a carboxy group, a cyano group, a nitro group, and a C1 to C5 alkoxy group.

When any of R^{1a} to R^{5a} is an ester group (—COOR), specific examples of the hydrocarbon moiety (R) of the ester group include the above-exemplified, substituted or unsubstituted alkyl groups and substituted or unsubstituted cycloalkyl groups. When any of R^{1a} to R^{5a} is an ester group, R^{1a} to R^{5a} are preferably methoxycarbonyl, ethoxycarbonyl, or n-butoxycarbonyl.

When any of R^{1a} to R^{5a} is an alkylsulfonyl group, examples of the alkyl moiety forming the alkylsulfonium group include the above-exemplified substituted or unsubstituted alkyl groups. When any of R^{1a} to R^{5a} is a cycloalkylsulfonyl group, examples of the alkyl moiety forming the cycloalkylsulfonium group include the aforementioned, substituted or unsubstituted cycloalkyl groups. When R^{1a} and R^{2a} represent a divalent group linked to a ring formed by combining R^{1a} and R^{2a}, R^{1a} and R^{2a} preferably form a single bond, —O—, —S—, —CO—, or —SO—.

In the aforementioned formula (6), each of R^{6a} and R^{7a} is preferably a substituted or unsubstituted C1 to C20 monovalent hydrocarbon group, —OR^{k}, —COOR^{k}, —O—CO—R^{k}, —O—R^{kk}—COOR^{k}, —R^{kk}—CO—R^{k}, —OSO₂—R^{k}, or —SO₂—R^{k} (wherein R^{k} represents a substituted or unsubstituted C1 to C20 monovalent hydrocarbon group, and R^{kk} represents a substituted or unsubstituted C1 to C20 divalent hydrocarbon group). Examples of the C1 to C20 monovalent hydrocarbon group represented by any of R^{6a}, R^{7a}, and R^{k} include the same groups as exemplified in relation to C1 to C20 monovalent hydrocarbon groups represented by R¹³ or the like in the aforementioned formulas (i-1) to (i-3). In R^{6a}, R^{7a}, and R^{k}, examples of the substituent which replaces a hydrogen atom of a hydrocarbon group include the same groups as exemplified in relation to substituents included in the aforementioned R^{3a}. Examples of R^{kk} include a group formed by removing one hydrogen atom from a substituted or unsubstituted C1 to C20 monovalent hydrocarbon group exemplified as R^{6a} or the like.

Examples of the divalent organic group represented by R^{8a} include a group formed by removing one hydrogen atom from, for example, a C1 to C20 monovalent organic group exemplified in relation to R^{6a} and R^{7a}.

Each of R^{6a} and R^{7a} is preferably, among the aforementioned groups, an unsubstituted, linear-chain or branched monovalent alkyl group, a monovalent fluoroalkyl group, an unsubstituted monovalent aromatic hydrocarbon group, —OSO₂—R^{k}, or —SO₂—R^{k}. The number a6 is preferably an integer of 0 to 2, more preferably 0 or 1, still more preferably 0. The number a7 is preferably an integer of 0 to 2, more preferably 0 or 1, still more preferably 0. The number t2 is preferably 0, and the number t1 is preferably 2 or 3, more preferably 2.

In the aforementioned formula (7), examples of the monovalent organic group represented by any of R^{9a} to R^{12a} include the same groups as exemplified in relation to the monovalent organic group represented by R¹ in the aforementioned formula (1b).

Specific examples of M⁺ include, but are not limited to, cations represented by the following formulas.

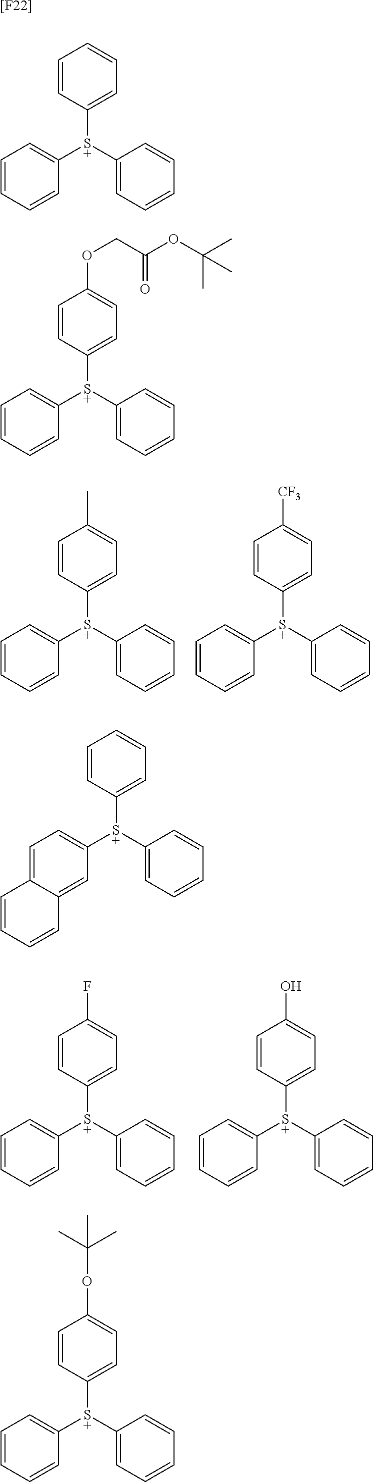

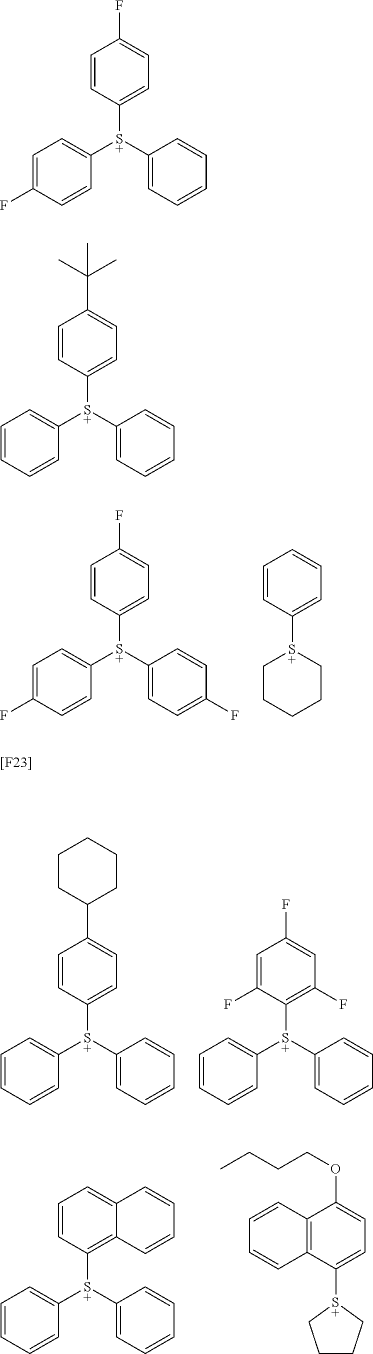

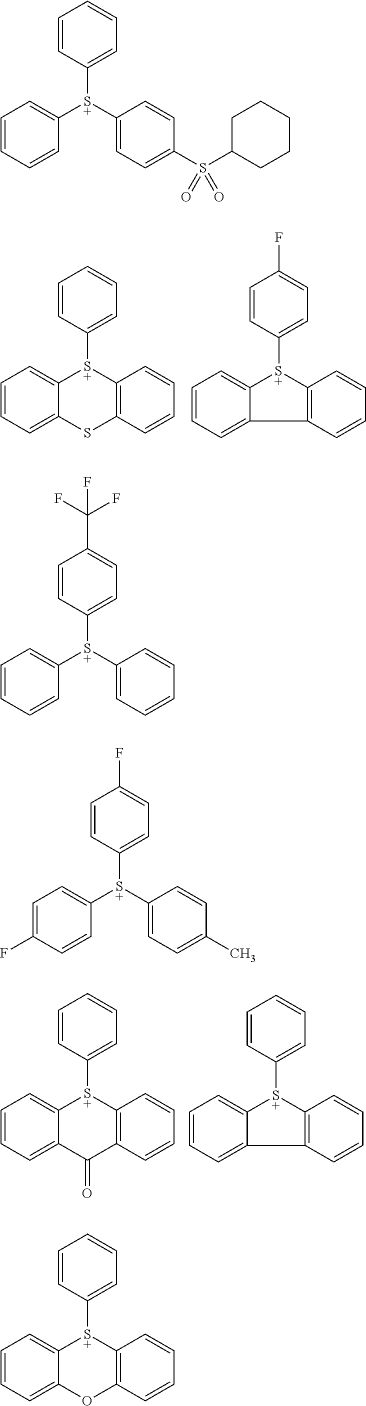

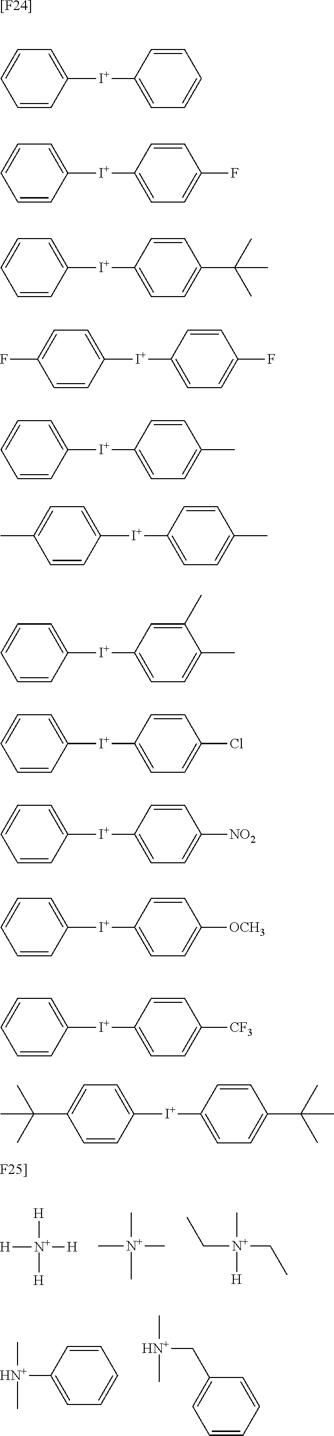

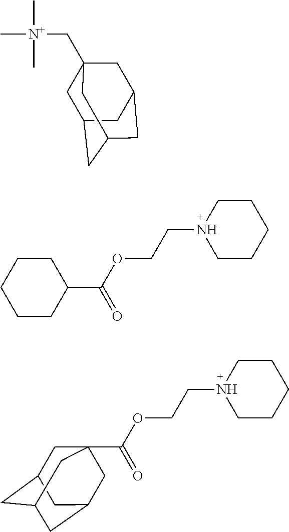 [SMILES: C[N+](C)(C)CC12CC3CC(CC(C3)C1)C2 ; O=C(OCCN1CCCCC1)C1CCCCC1 ; O=C(OCCN1CCCCC1)C12CC3CC(CC(C3)C1)C2]

Specific examples of the compound (CB) include compounds represented by the following formulas (b-1) to (b-41). In the following formulas (b-1) to (b-41), M⁺ represents a monovalent cation. Examples of the monovalent cation represented by M⁺ are the same as exemplified above.

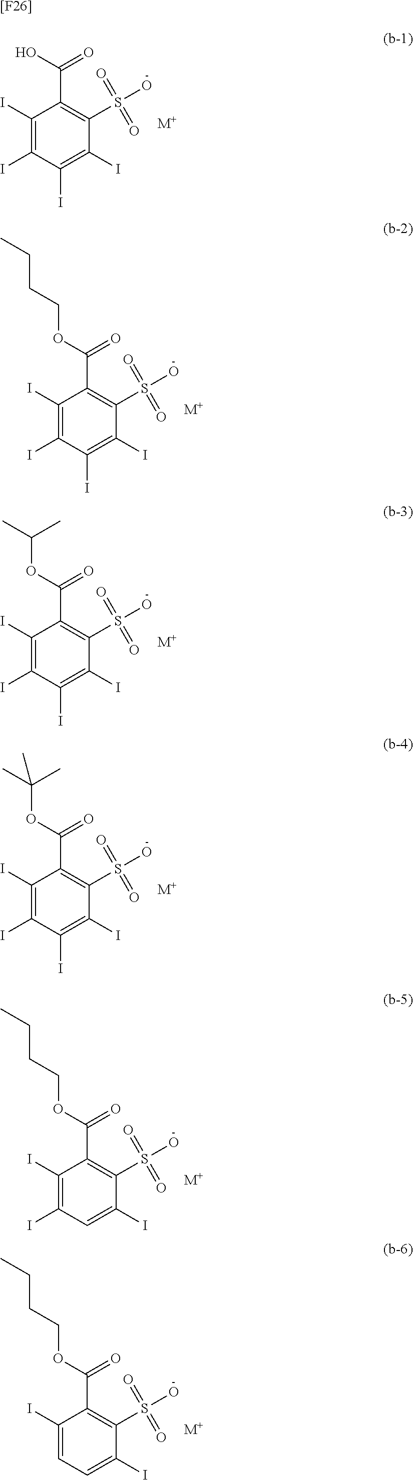

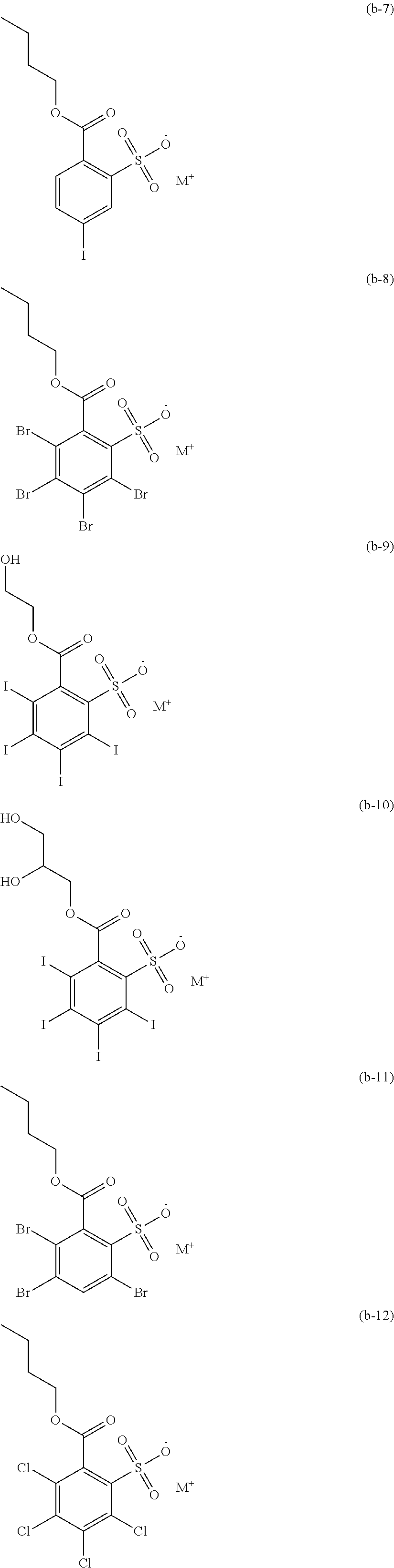

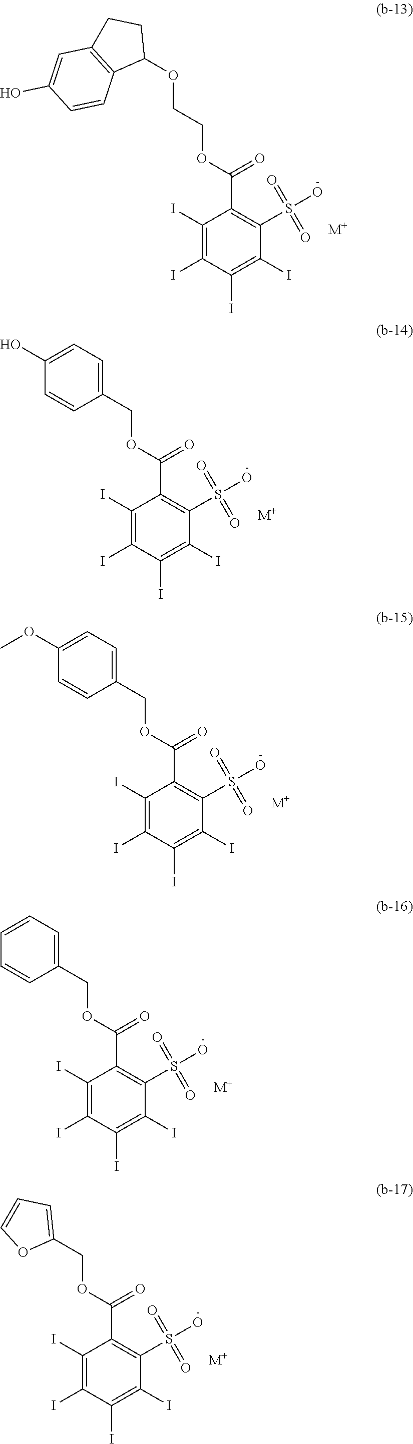

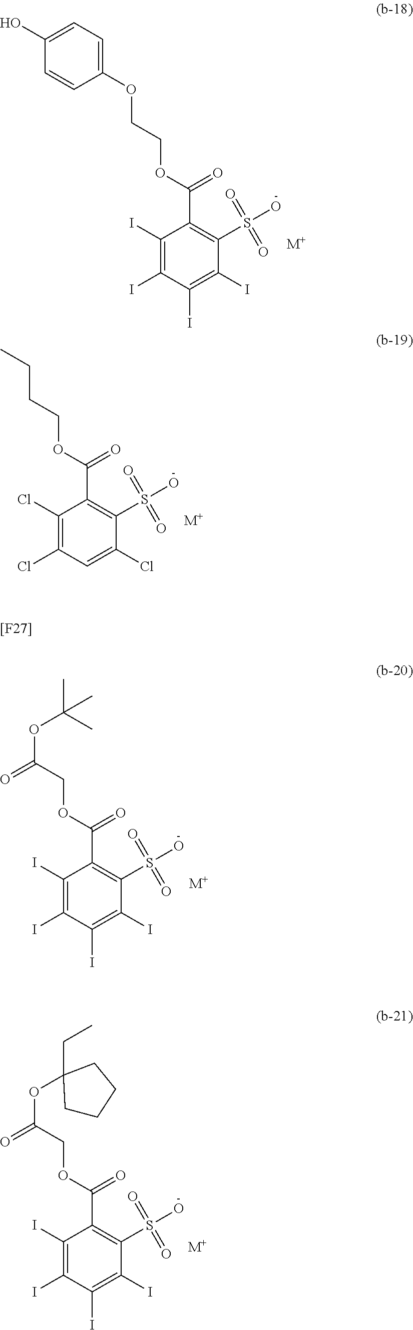

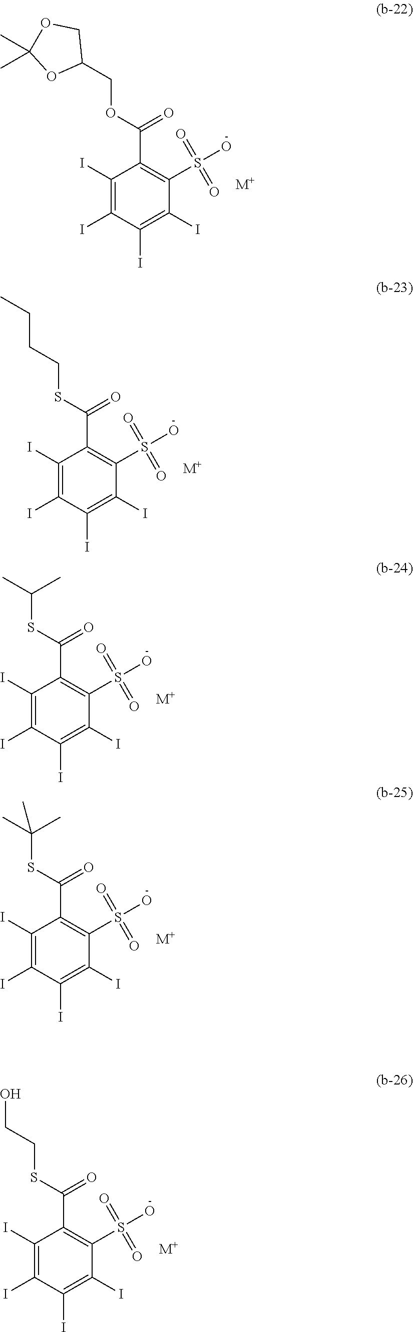

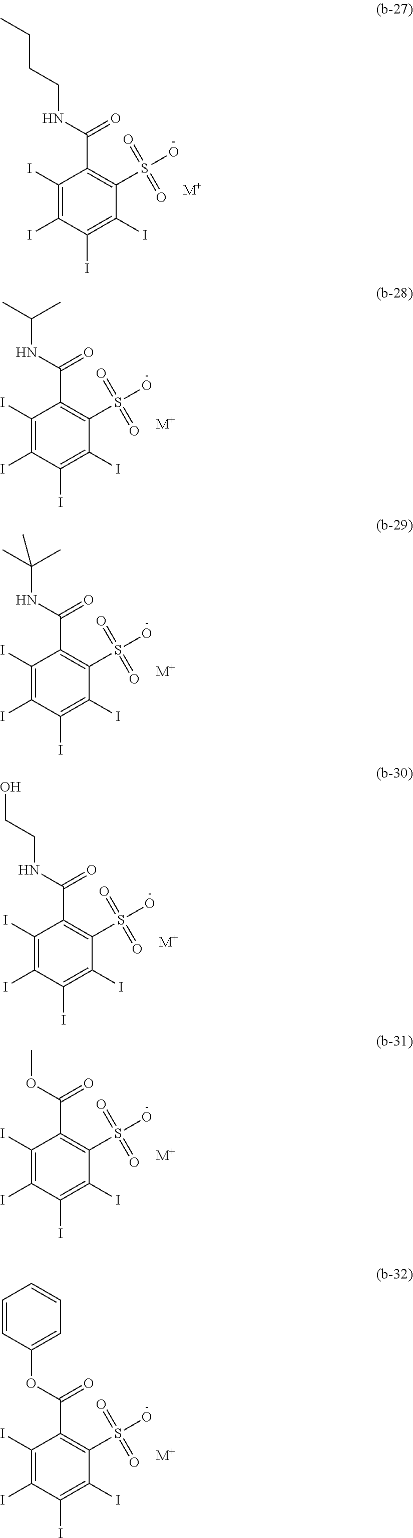

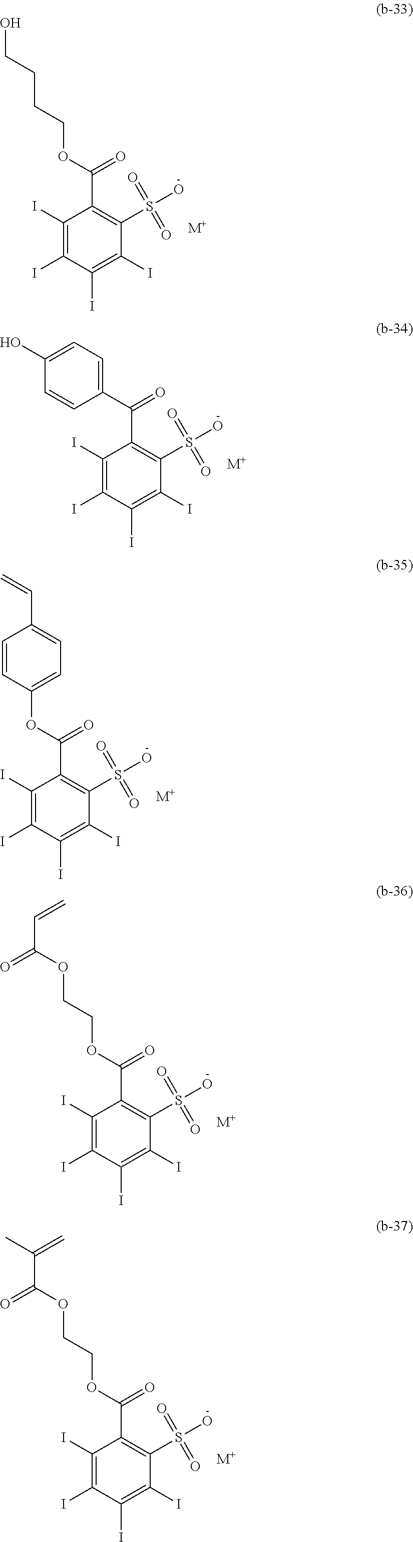

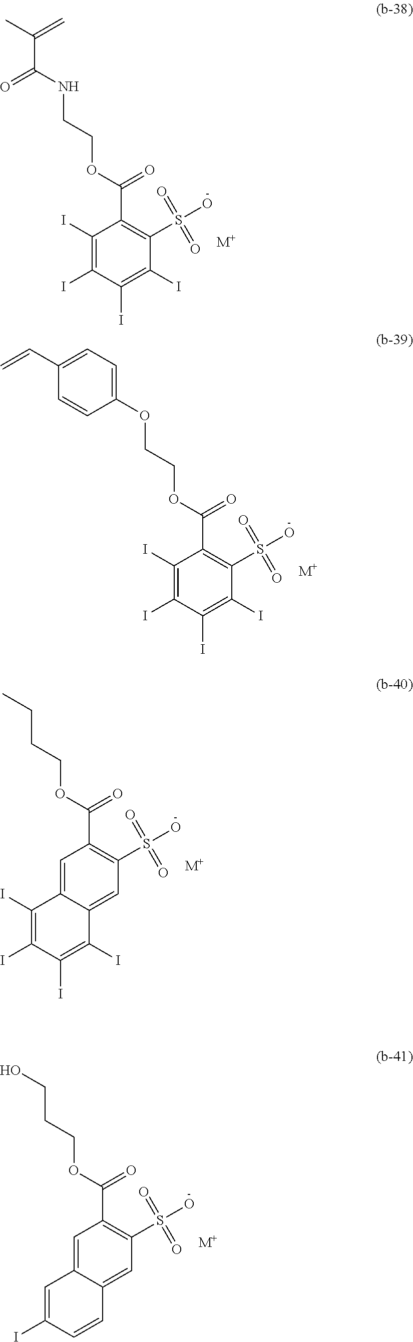

In the first composition, the relative amount of the compound (CB) with respect to 100 parts by mass of the polymer (A) is preferably 1 part by mass or more, more preferably 2 parts by mass or more, still more preferably 5 parts by mass or more. Also, the relative amount of the compound (CB) with respect to 100 parts by mass of the polymer (A) is preferably 40 parts by mass or less, more preferably 35 parts by mass or less, still more preferably 30 parts by mass or less. Controlling the compound (CB) content to satisfy the above conditions is preferred, since the effects of improving the sensitivity and LWR performance of the first composition and securing a wide process window can be achieved in a well-balanced manner. The compound (CB) may be used singly or in combination of two or more species.

### Synthesis of Compound (CB)

The compound (CB) can be synthesized through customary methods of organic chemistry in appropriate combinations. A specific procedure of the method of synthesizing the compound (CB) will be described, taking the case where A¹ is —O— as an example. According to the following schemes, an acid anhydride represented by the following formula (3) is reacted with a compound having a hydroxy group (R¹—OH) in an appropriate solvent in the presence of a base. Subsequently, the thus-formed intermediate is reacted with a sulfonium chloride or the like which can provide an onium cation moiety, to thereby yield the compound (CB). In an alternative procedure, when A¹ is —S— or —NR³—, the compound (CB) can be synthesized through the same technique employing a compound having a thiol group or a compound having an amino group. When A¹ is a single bond, the first reaction is performed in the presence of a Lewis acid, and the thus-obtained intermediate is reacted with a sulfonium chloride or the like which can provide an onium cation moiety, to thereby yield the compound (CB). The method of synthesizing the compound (CB) is not limited to the aforementioned routes. In the following schemes, “TPSCl” denotes triphenylsulfonium chloride (the same applies hereinafter).

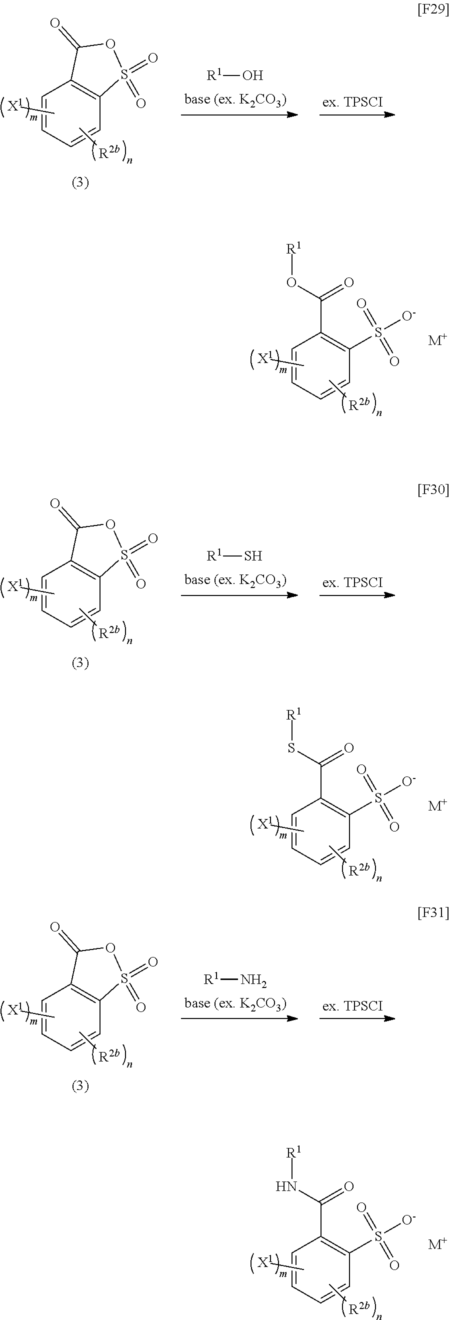

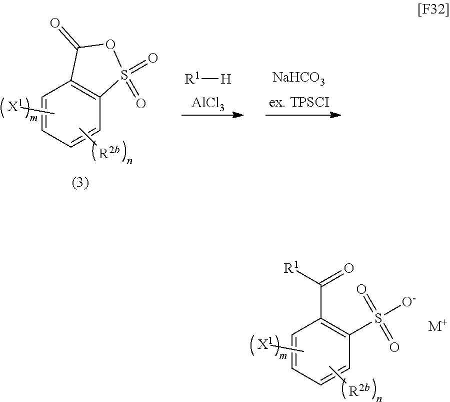

### [Additional Acid-Generating Agent]

No particular limitation is imposed on the additional acid-generating agent, and a known radiation-sensitive acid-generating agent employable for formation of a resist pattern may be used. As the additional acid-generating agent, an onium salt compound formed of a radiation-sensitive onium cation and an organic anion is preferably used.

The cation included in the additional acid-generating agent preferably has a sulfonium cation structure or an iodonium cation structure, more preferably a triarylsulfonium cation structure or a diaryliodonium cation structure. Specific examples of the cation forming the additional acid-generating agent include cations as exemplified in relation to the cations represented by the aforementioned formula (4) or (5).

The organic anion included in the additional acid-generating agent is preferably a sulfonate anion, an imide anion, or a methide anion, more preferably a sulfonate anion. From the viewpoint of achieving higher sensitivity of the first composition, the organic anion included in the additional acid-generating agent preferably has an iodo group. Among them, a sulfonate anion having an iodo group is preferred, with a sulfonate anion having a structure in which an iodine atom is bound to an aromatic ring being more preferred.

Specific examples of the additional acid-generating agent include onium salt compounds represented by the following formulas. The additional acid-generating agent is not limited to the compounds having the following structures.

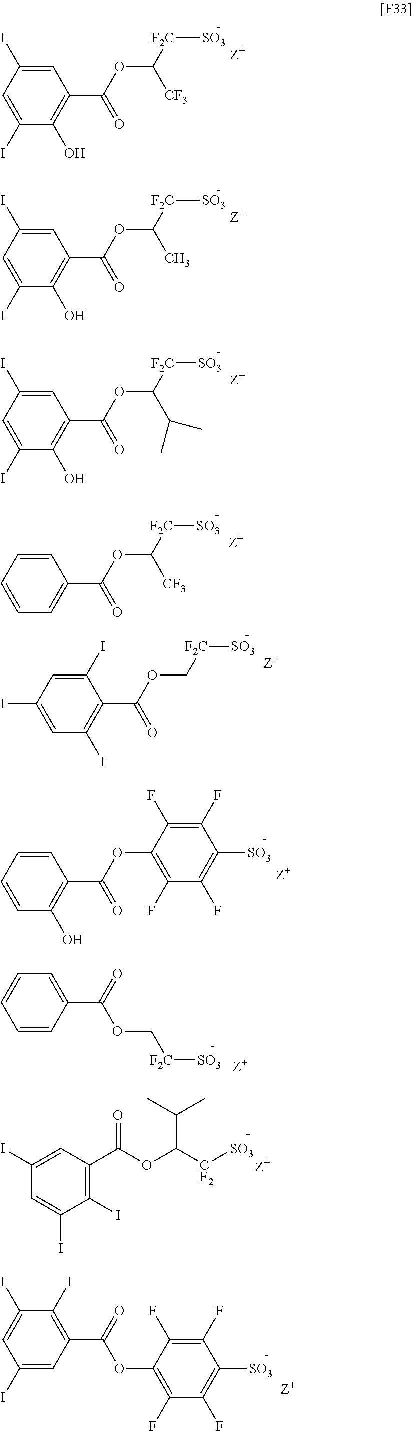

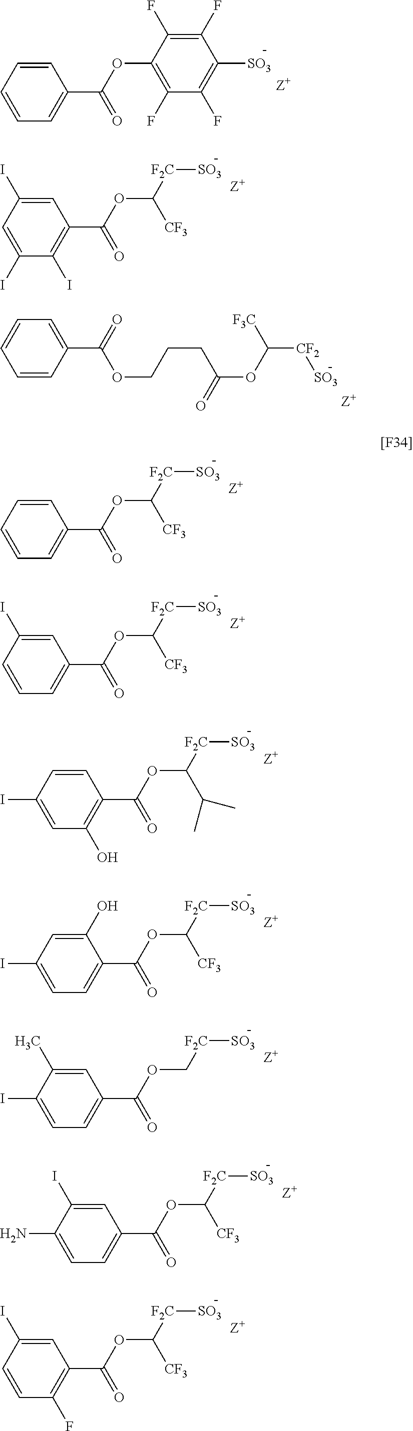

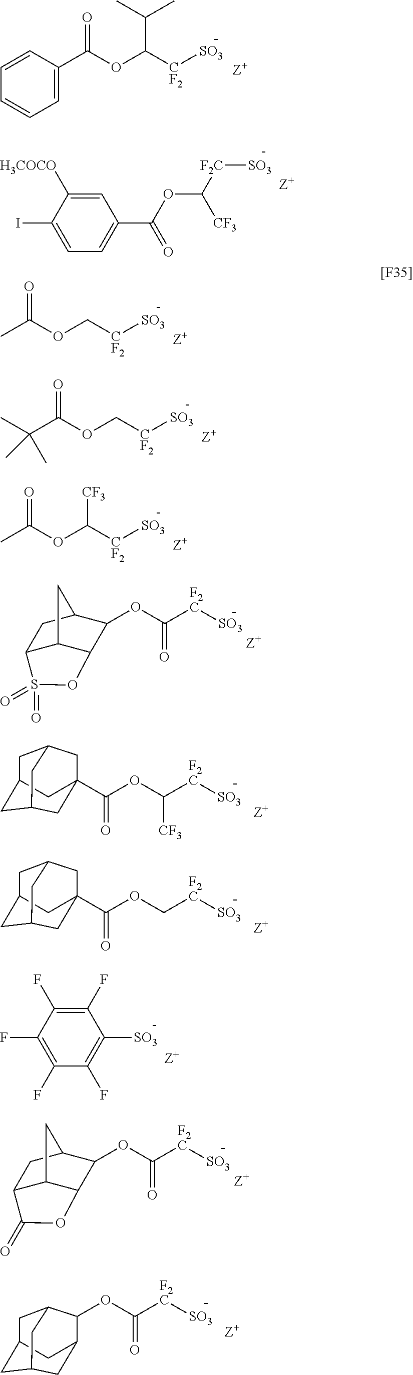

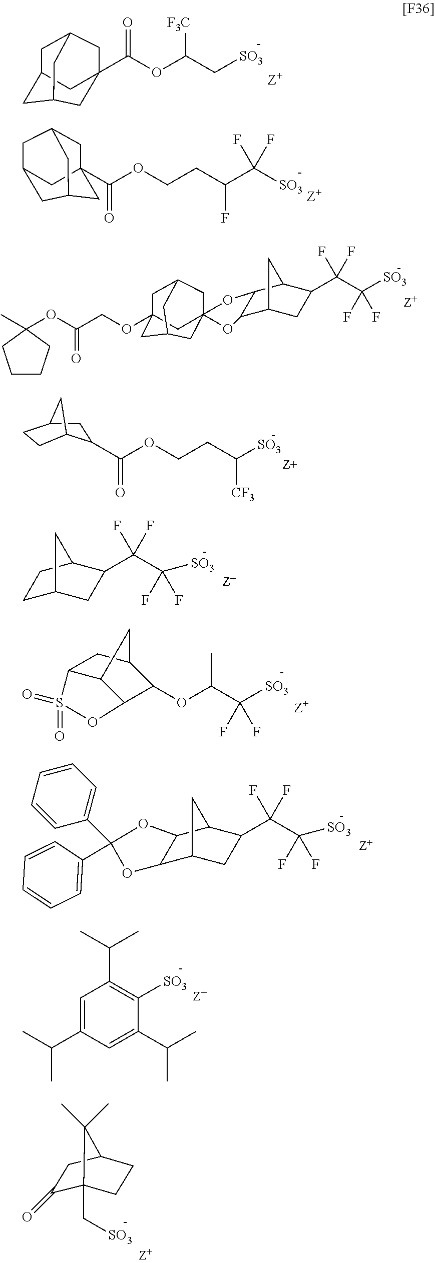

When the additional acid-generating agent is incorporated into the first composition, the relative amount of the additional acid-generating agent, with respect to 100 parts by mass of the polymer (A), is preferably 0.5 parts by mass or more, more preferably 1 part by mass or more, still more preferably 2 parts by mass or more. Also, the relative amount of the additional acid-generating agent, with respect to 100 parts by mass of the polymer (A), is preferably 20 parts by mass or less, more preferably 15 parts by mass or less, still more preferably 10 parts by mass or less. In addition, when the additional acid-generating agent is employed, preferably, the acid-generating agent content is adjusted to be lower than the compound (CB) content. Specifically, the ratio in amount of the additional acid-generating agent to the sum of the amount of the compound (CB) and that of the acid-generating agent is preferably adjusted to less than 50 mass %, more preferably 35 mass % or less. Notably, the additional acid-generating agent may be used singly or in combination of two or more species.

### [Acid Diffusion Control Agent]

The acid diffusion control agent is a component that can suppress chemical reaction by an acid in the light-unexposed part, with a mechanism of suppressing diffusion of the acid in a resist film, the acid generated in the resist film through light exposure. By incorporating the acid diffusion control agent into the first composition, lithographic characteristics (LWR performance and CDU performance) of the first composition can be further enhanced. Furthermore, variation in line width of a resist pattern, which would otherwise be caused by variation in post-exposure time (i.e., duration of time from light exposure to development) can be suppressed, whereby a radiation-sensitive composition having excellent process stability can be obtained. Examples of the acid diffusion control agent include a nitrogen-containing compound and a light-degradable base.

### (Nitrogen-Containing Compound)

As the nitrogen-containing compound, a known nitrogen-containing compound employed for forming a resist pattern may be used. Specific examples of the nitrogen-containing compound include an amide group-containing compound (e.g., alkylamine, aromatic amine, or polyamine), an amino group-containing compound, a urea compound, a nitrogen-containing heterocyclic compound (e.g., N-(undecan-1-yl carbonyloxyethyl) morpholine), a nitrogen-containing compound having an acid-releasable group (e.g., N-(t-butoxycarbonyl)di-n-octylamine, or N-t-butoxycarbonyl-4-hydroxypiperidine). The nitrogen-containing compound may be used singly or in combination of two or more species.

### (Light-Degradable Base)

The light-degradable base is a compound which generates an acid weaker than the radiation-sensitive acid-generating agent upon light exposure. In the present disclosure, the light-degradable base is a compound which generates an acid weaker than the compound (CB) upon light exposure. Meanwhile, the degree of acidity can be assessed on the basis of acid dissociation constant (pKa). The acid dissociation constant (pKa) of the acid generated by the action of the light-degradable base is generally −3 or higher, preferably −1 to 7, more preferably 0 to 5. Under general conditions, the acid generated by the light-degradable base is a weak acid which does not evoke release of an acid-releasable group. As used herein, the term “general conditions” refers to conducting post-exposure bake (PEB) at 110 °C for 60 seconds.

As the light-degradable base, an onium salt compound formed of a radiation-sensitive onium cation and an organic anion is preferably used. From the viewpoint of enhancing lithographic characteristics of the first composition, the light-degradable base is preferably, among others, an onium salt that can generate carboxylic acid, sulfonic acid, or sulfonamide through light exposure. Also, from the viewpoint of successfully forming a resist film exhibiting higher LWR performance, an onium salt having a sulfonium cation structure or an iodonium cation structure is preferably used as a light-degradable base. The light-degradable base may be used singly or in combination of two or more species.

Specific examples of the light-degradable base include onium salt compounds represented by the following formula (9).

### [F37]

E⁻U⁺ (9)

(In the formula (9), E⁻ represents an organic anion represented by “R⁵¹—COO⁻”, “R⁵²—SO₂—N⁻—R⁵¹,” or “R⁵¹—SO₃⁻”; each of R⁵¹ and R⁵² independently represents a C1 to C30 monovalent organic group; when E⁻ is an organic anion represented by “R⁵¹—SO₃⁻”, no fluorine atom is bound to a carbon atom to which “SO₃⁻” is bound; and Ut represents a radiation-sensitive onium cation.)

In the aforementioned formula (9), examples of the C1 to C30 monovalent organic group represented by R⁵¹ include a substituted or unsubstituted C1 to C30 monovalent hydrocarbon group; a C1 to C30 monovalent group containing a hetero atom-containing divalent group in a carbon-carbon bond of the hydrocarbon group or at an end on the chemical bond side (hereinafter may also be referred to as a “monovalent group γ”); and a monovalent group in which at least one hydrogen atom of the monovalent group γ is substituted. Examples of the substituent in the substituted hydrocarbon group or the substituted monovalent group γ include a fluorine atom, a chlorine atom, a bromine atom, an iodine atom, a hydroxy group, an aldehyde group, an acetyl group, and an ester group. The C1 to C30 monovalent organic group represented by R⁵¹ is preferably, among others, a monovalent group having a substituted or unsubstituted aromatic ring.

Examples of the C1 to C30 monovalent organic group represented by R⁵² include a substituted or unsubstituted alkyl group, and a substituted or unsubstituted cycloalkyl group. Examples of the substituent of the substituted alkyl group include a fluorine atom. Examples of the substituent of the substituted cycloalkyl group include a C1 to C10 alkyl group, a fluorine atom, and an iodine atom.

The organic anion (E⁻) forming the light-degradable base preferably has a carboxylate anion structure or a sulfonate anion structure. From the viewpoint of achieving a high effect of improving lithographic characteristics (LWR performance) of the first composition, the organic anion (E⁻) preferably has an iodo group, more preferably an iodo group bound to an aromatic ring. The number of iodo group(s) belonging to the organic anion (E⁻) is essentially ≥1, preferably ≥2. From the viewpoint of solubility, the number of iodo group(s) belonging to the organic anion (E⁻) is, for example, ≤4.

The cation (U⁺) forming the light-degradable base preferably has a sulfonium cation structure or an iodonium cation structure, more preferably a triarylsulfonium cation or a diaryliodonium cation structure. Specific examples of the cation (U⁺) include the same cations as exemplified in relation to cations forming the compound (CB).

Specific examples of the light-degradable base include onium salt compounds represented by the following formula. However, the light-degradable base is not limited to these onium salt compounds. In the following formulas, U⁺ represents a radiation-sensitive onium cation.

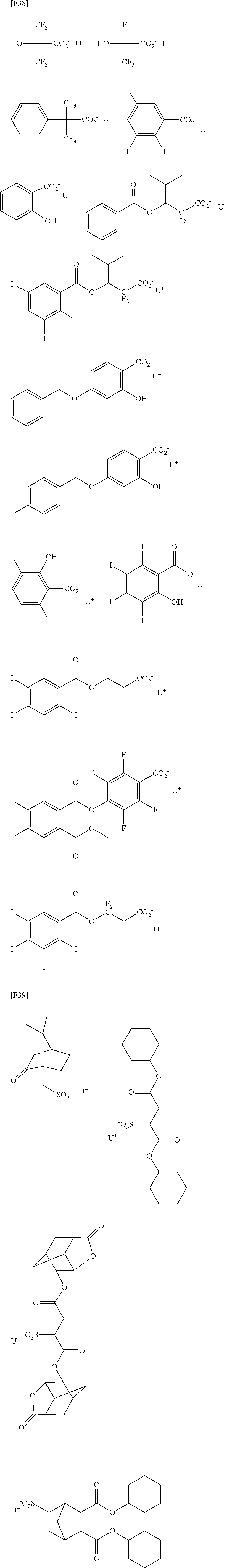

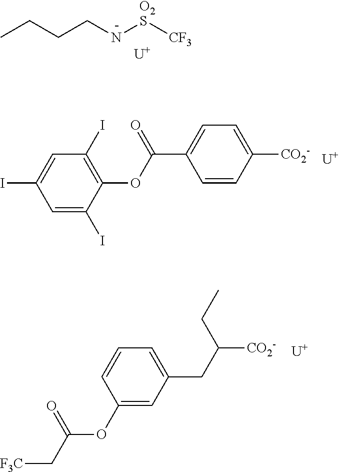

Also, the first composition may contain, as a light-degradable base, a compound in which L⁻ in the aforementioned formula (1) is —COO⁻. Specific examples of the compound include compounds represented by the following formulas (d-1) to (d-45).

When the first composition contains an acid diffusion control agent, the relative amount of the acid diffusion control agent in the first composition, with respect to the amount of the radiation-sensitive acid-generating agent (the compound (CB) and the additional acid-generating agent) contained in the first composition (the total amount when two or more members are used), is preferably 1 mol % or more, more preferably 2 mol % or more, still more preferably 5 mol % or more. Also, the relative amount of the acid diffusion control agent, with respect to the amount of the radiation-sensitive acid-generating agent contained in the first composition, is preferably 50 mol % or less, more preferably 40 mol % or less, still more preferably 35 mol % or less. By adjusting the acid diffusion control agent content to satisfy the above conditions, LWR performance of the first composition can be further enhanced. The acid diffusion control agent may be used singly or in combination of two or more species.

### [Solvent]

No particular limitation is imposed on the solvent, so long as the solvent can dissolve or disperse the components incorporated into the first composition therein. Examples of the solvent include an alcohol, an ether, a ketone, an amide, an ester, and a hydrocarbon.

Examples of the alcohol include C1 to C18 aliphatic monoalcohols such as 4-methyl-2-pentanol and n-hexanol; C3 to C18 alicyclic monoalcohols such as cyclohexanol; C2 to C18 polyhydric alcohols such as 1,2-propylene glycol; and C3 to C19 polyhydric alcohol partial ethers such as propylene glycol monomethyl ether. Examples of the ether include dialkyl ethers such as diethyl ether, dipropyl ether, dibutyl ether, dipentyl ether, diisoamyl ether, dihexyl ether, and diheptyl ether; cyclic ethers such as tetrahydrofuran and tetrahydropyran; and aromatic ring-containing ethers such as diphenyl ether and anisole.

Examples of the ketone include chain ketones such as acetone, methyl ethyl ketone, methyl n-propyl ketone, methyl n-butyl ketone, diethyl ketone, methyl isobutyl ketone, 2-heptanone, ethyl n-butyl ketone, methyl n-hexyl ketone, diisobutyl ketone, and trimethylnonanone; cyclic ketones such as cyclopentanone, cyclohexanone, cycloheptanone, cyclooctanone, and methylcyclohexanone; and 2,4-pentanedione, acetonylacetone, acetophenone, and diacetone alcohol. Examples of the amide include cyclic amides such as N,N′-dimethylimidazolidinone and N-methylpyrrolidone; and chain amides such as N-methylformamide, N, N-dimethylformamide, N, N-diethylformamide, acetamide, N-methylacetamide, N, N-dimethylacetamide, and N-methylpropionamide.

Examples of the ester include monocarboxylic acid esters such as n-butyl acetate and ethyl lactate; polyhydric alcohol carboxylates such as propylene glycol acetate; polyhydric alcohol partial ester carboxylates such as propylene glycol-1-monomethyl ether acetate; polybasic carboxylic acid diesters such as diethyl oxalate; carbonates such as dimethyl carbonate and diethyl carbonate; and cyclic esters such as γ-butyrolactone. Examples of the hydrocarbon include C5 to C12 aliphatic hydrocarbons such as n-pentane and n-hexane; and C6 to C16 aromatic hydrocarbons such as toluene and xylene.

Among the above solvents, the solvent preferably includes at least one member selected from the group consisting of the ester and the ketone, more preferably at least one member selected from the group consisting of polyhydric alcohol partial ether carboxylates and cyclic ketones, still more preferably at least one member among propylene glycol monomethyl ether acetate, ethyl lactate, and cyclohexanone. The solvent may be used singly or in combination of two or more species.

### [High-Fluorine Content Polymer]

The high-fluorine content polymer (hereinafter may also be referred to simply as a “polymer (F)”) is a polymer having a fluorine atom content (by mass) greater than that of polymer (A). When the first composition contains the polymer (F), the polymer (F) can be localized on the surface of a resist film with respect to the polymer (A). As a result, water repellency of the surface of the resist film can be enhanced in exposure to light in a liquid immersion mode.

No particular limitation is imposed on the fluorine atom content of the polymer (F), so long as it is greater than the fluorine atom content of the polymer (A). The fluorine atom content of the polymer (F) is preferably 1 mass % or more, more preferably 2 mass % or more, still more preferably 4 mass % or more. Also, the fluorine atom content of the polymer (F) is preferably 60 mass % or less, more preferably 40 mass % or less, still more preferably 30 mass % or less. The fluorine atom content (mass %) of a polymer can be obtained by determining the structure of the polymer through ¹³C-NMR spectrometry or the like and calculating the content based on the structure determined.

The Mw of the polymer (F), which is determined through GPC, is preferably 1,000 or more, more preferably 3,000 or more, still more preferably 4,000 or more. Also, the Mw of the polymer (F) is preferably 50,000 or less, more preferably 30,000 or less, still more preferably 20,000 or less. The molecular weight distribution parameter represented by a ratio of Mw to Mn (Mw/Mn) of the polymer (F), which is determined through GPC, is preferably 1 to 5, more preferably 1 to 3.

The polymer (F) content of the first composition, with respect to 100 parts by mass of the polymer (A), is preferably 10 parts by mass or less, more preferably 5 parts by mass or less.

### [Additional and Optional Component]

The first composition may further contain a component which differs from the aforementioned polymer (A), the compound (CB), the additional acid-generating agent, the acid diffusion control agent, a solvent, and polymer (F) (hereinafter may also be referred to as “additional and optional component”). Examples of the additional and optional component include a surfactant, a compound having an alicyclic skeleton (e.g., 1-adamantanecarboxylic acid, 2-adamantanone, or t-butyl deoxycholate), and a sensitizer, a localization accelerator. So long as the effects of the present disclosure are not impaired, the additional and optional component content of the first composition may be appropriately set depending on the property of respective components.

### [Method of Producing Radiation-Sensitive Composition]

The first composition may be produced through, for example, the following procedure: mixing the polymer (A) and the compound (CB) with an optional component such as the solvent at a desired ratio and filtering the resultant mixture preferably by means of a filter (e.g., a filter having a pore size of about 0.2 μm) or the like. The solid content of the first composition is preferably 0.1 mass % or more, more preferably 0.5 mass % or more, still more preferably 1 mass % or more. Also, the solid content of the first composition is preferably 50 mass % or less, more preferably 20 mass % or less, still more preferably 5 mass % or less. Adjusting the solid content of the first composition to satisfy the above conditions is preferred, since coatability of the composition can be enhanced, to thereby obtain a resist pattern having a suitable shape.

### <Second Composition>

Next, a radiation-sensitive composition of a second embodiment according to the first radiation-sensitive composition (i.e., a second composition) will be described. Among the members of the second compositions, descriptions of the same members as those of the first composition are omitted.

The second composition contains the polymer (PB). The polymer (PB) may serve as a base resin of the second composition or a component which serves as a radiation-sensitive acid-generating agent and which is incorporated separately with the base resin. In a preferred mode, the second composition contains the polymer (PB) as a base resin.

In the aforementioned formula (2b), A¹, X¹, m, n, R², and M⁺ are the same as described in relation to the aforementioned formula (1b). Examples of the divalent organic group represented by R⁴ include a group formed by removing any one hydrogen atom from a group exemplified as the monovalent organic group represented by R¹.

The polymer (PB) preferably includes a structural unit having a partial structure represented by the aforementioned formula (2b) (hereinafter may also be referred to as a “structural unit (VI)”). Specific examples of preferred the structural units (VI) include those represented by the following formula (2b-1) or (2b-2).

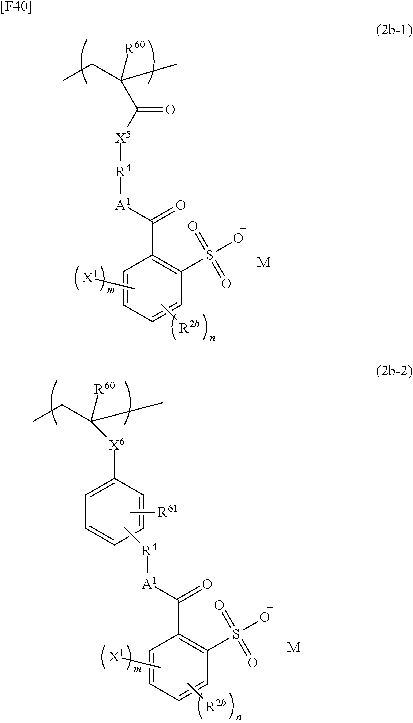

(In the formulas (2b-1) and (2b-2), R⁶⁰ represents a hydrogen atom, a fluorine atom, a methyl group, or a trifluoromethyl group; X⁵ represents —O— or —NH—; X⁶ represents a single bond, —COO—, or —CONH—; R⁶¹ is a monovalent substituent; s is an integer of 0 to 4; and A¹, X¹, m, n, R^{2b}, R⁴, and M⁺ have the same meanings as those defined in the aforementioned formula (2b).)

In the aforementioned formulas (2b-1) and (2b-2), specific examples and of A¹, X¹, m, n, R^{2b}, and M⁺, and preferred members thereof are the same as those described in relation to the aforementioned formula (1b). Examples of the divalent organic group represented by R⁴ include a group formed by removing any one hydrogen atom from a monovalent organic group exemplified as R¹ in the aforementioned formula (1b). Examples of the monovalent substituent represented by R⁶¹ include a C1 to C3 alkyl group, a C1 to C3 alkoxy group, a hydroxy group, and a halogen atom. The parameter s is preferably 0 to 2, more preferably 0 or 1.

Specific examples of the structural unit (VI) include structural units which are represented by any of the aforementioned formulas (v-1) to (v-5) which are specifically exemplified as the structural unit (V).

In the polymer (PB), the relative amount of structural unit (VI) in all the structural units forming the polymer (PB) is preferably 1 mol % or more, more preferably 3 mol % or more, still more preferably 5 mol % or more. Also, the relative amount of structural unit (VI) in all the structural units forming the polymer (PB) is preferably 50 mol % or less, more preferably 40 mol % or less, still more preferably 30 mol % or less. Adjusting the structural unit (VI) content to satisfy the above conditions is preferred, since lithographic characteristics such as LWR performance can be enhanced while high sensitivity is maintained, and a wide process window can be secured.

The polymer (PB) may further include a structural unit other than the structural unit (VI). Examples of the additional structural unit include structural units (I) to (IV), described as those in relation to the polymer (A) contained in the first composition, and other structural units. Descriptions of the relative amount of any of the structural units in the polymer (PB) are the same as those in relation to the polymer (A). When the second composition contains the polymer (PB) as a base resin, the polymer (PB) preferably has an acid-releasable group, more preferably the structural unit (I). Also, the polymer (PB) may be a polymer which includes the structural unit (VI) and which has an acid-releasable group and a structural unit having an onium salt structure formed of a radiation-sensitive cation and a carboxylic acid anion.

Examples of the optional component incorporated into the second composition include the additional acid-generating agent, the acid diffusion control agent, the high-fluorine content polymer, and the solvent, which have been described in relation to the first composition. Specific examples thereof, preferred embodiments thereof, relative amounts of the components, etc. are the same as those of the first composition.

The first radiation-sensitive composition (including the first composition and the second composition) prepared through the aforementioned procedure may be used as a positive-type pattern formation composition in formation of a pattern by use of an alkaline developer, or a negative-type pattern formation composition in formation of a pattern by use of a developer containing an organic solvent. The present composition is particularly suitable as a radiation-sensitive composition for EUV light exposure which is used for forming a resist pattern through exposure to extreme UV (EUV) light.

### <Method of Forming Resist Pattern>

The resist pattern formation method of the present disclosure includes a step of applying the present composition on one surface of a substrate (hereinafter may also be referred to as a “application step”), a step of exposing to light a resist film obtained in the application step (hereinafter may also be referred to as a “light-exposure step”), and a step of developing the light-exposed resist film (hereinafter may also be referred to as a “development step”). Examples of the pattern obtained through the resist pattern formation method of the present disclosure include a line-and-space pattern and a hole pattern. Since a resist film is formed by use of the present composition in the resist pattern formation method of the present disclosure, a resist pattern which exhibits excellent sensitivity and lithographic characteristics and which has few development faults can be formed. The steps will next be described in detail.

### [Application Step]

In this step, the present composition is applied onto one surface of a substrate, to thereby form a resist film on the substrate. A conventionally known substrate can be used as a substrate on which resist film is to be formed. Examples of the substrate include a silicon wafer and a wafer coated with silicon dioxide or aluminum. For example, an organic or inorganic anti-reflection film disclosed in, for example, Japanese Patent Publication (kokoku) No. 1994-12452 or Japanese Patent Application laid-Open (kokai) No. 1984-93448 may be formed on a substrate to be used. Examples of the method of applying the present composition include spin coating, flow casting, and roller coating. After application, the applied composition may be subjected to pre-baking (PB) so as to evaporate the solvent remaining in the coating film. The temperature of PB is preferably 60 °C or higher, more preferably 80 °C or higher. Also, the temperature of PB is preferably 140 °C or lower, more preferably 120 °C or lower. The time of PB is preferably 5 seconds or longer, more preferably 10 seconds or longer, and preferably 600 seconds or shorter, more preferably 300 seconds or shorter. The average thickness of the formed resist film is preferably 10 to 1,000 nm, more preferably 20 to 500 nm.

When liquid immersion light exposure is conducted in the subsequent exposure step, a liquid immersion protective film which is insoluble in an immersion liquid may further be provided on the resist film formed from the present composition in order to prevent direct contact between the immersion liquid and the resist film, regardless of addition of a water-repellent polymer additive such as the polymer (F) in the present composition is conducted. As the liquid immersion protective film, there may be employed any of a solvent-removable protective film which is removed by solvent before conducting the development step (see, for example, Japanese Paten Application Laid-Open (kokai) No. 2006-227632), and a developer-removable protective film which is removed simultaneously with the development step (see, for example, WO 2005/069076 and WO 2006/035790). From the viewpoint of through-put, a developer-removable immersion liquid protective film is preferably employed.

### [Light-Exposure Step]

In this step, the resist film formed through the above application step is exposed to light. In the light exposure, the resist film is irradiated with radiation by the mediation of a photomask or, in some cases, a liquid immersion medium such as water. The radiation is selected in accordance with the line width of a target pattern, and examples thereof include electromagnetic waves such as visible light, a near-UV ray, a far-UV ray, an extreme UV (EUV) ray, an X-ray, and a γ-ray; and charged particle rays such as an electron beam and an α-ray. Among them, the radiation applied to the resist film formed from the present composition is preferably a far-UV ray, an EUV ray, or an electron beam, more preferably ArF excimer laser light (wavelength: 193 nm), KrF excimer laser light (wavelength: 248 nm), an EUV ray, or an electron beam, still more preferably ArF excimer laser light, an EUV ray, or an electron beam, particularly preferably an EUV ray.

After completion of the above light exposure, preferably, post exposure baking (PEB) is performed so as to accelerate dissociation of an acid-releasable group, wherein dissociation is mediated by an acid generated by the radiation-sensitive acid-generating agent through light exposure in the light-exposed part of the resist film. Through PEB, the difference in dissolution performance with respect to a developer between the exposed part and the unexposed part can be increased. The temperature at PEB is preferably 50 °C or higher, more preferably 80 °C or higher, and preferably 180 °C or lower, more preferably 130 °C or lower. The time of PEB is preferably 5 seconds or longer, more preferably 10 seconds or longer, and preferably 600 seconds or shorter, more preferably 300 seconds or shorter.

### [Development Step]

In the development step, the resist film exposed to light in the above step is developed with a developer. Thus, a resist pattern of interest can be formed. Either an alkaline developer or an organic solvent developer may be used as the developer. The developer may be appropriately selected in accordance with a target pattern (i.e., positive pattern or negative pattern).

Examples of the developer employed in the alkali development include aqueous alkaline solutions in which at least one species from among alkaline compounds such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (TMAH), pyrrole, piperidine, choline, 1,8-diazabicyclo[5.4.0]-7-undecene, and 1,5-diazabicyclo[4.3.0]-5-nonene is dissolved. Among such alkaline solutions, an aqueous TMAH solution is preferred, with 2.38-mass % aqueous TMAH solution being more preferred.

In the case of development with an organic solvent, examples of the developer include of organic solvents such as hydrocarbons, ethers, esters, ketones, and alcohols; and a solvent containing any of the organic solvents. Examples of organic solvents include one or more solvents listed as a solvent which can be incorporated into the present composition. Among them, an ether, an ester, and a ketone are preferred. The ether is preferably a glycol ether, more preferably ethylene glycol monomethyl ether or propylene glycol monomethyl ether. The ester is preferably an acetate ester, more preferably n-butyl acetate or amyl acetate. The ketone is preferably a chain ketone, more preferably 2-heptanone. The organic solvent content of the developer is preferably 80 mass % or more, more preferably 90 masse or more, still more preferably 95 mass % or more, particularly preferably 99 mass % or more. Examples of developer components other than organic solvent include water and silicone oil.

Examples of the development method include a dipping method (i.e., dipping a substrate in a bath filled with a developer for a specific time); a paddle method (i.e., putting a developer in a substrate to form a drop by surface tension and allowing the substrate to stand for a specific time); a spray method (i.e., spraying a developer onto a substrate); and a dynamic dispense method (i.e., continuously jetting a developer at a specific speed to a substrate rotating at a specific speed through a developer jetting nozzle with scanning). After development, generally, the substrate is washed with a rinse liquid (e.g., water or alcohol) and then dried.

The first radiation-sensitive composition described hereinabove contains the compound (CB) or the polymer (PB) or both. Thus, the composition provides high sensitivity in formation of a resist pattern and exhibits excellent LWR performance and a wide process window. Therefore, the present composition can be suitably used semiconductor device processing and the like, where further process shrinkage will conceivably proceed.

### [Second Radiation-Sensitive Composition]

Next, a second radiation-sensitive composition will be described. Regarding the second radiation-sensitive composition, the same descriptions as those of the first radiation-sensitive composition are incorporated herein by reference, and omitted in the specification.

The second radiation-sensitive composition contains a compound represented by the aforementioned formula (1) or (2) in which L⁻ is —COO⁻. When L⁻ is —COO⁻, the aromatic hydrocarbon ring forming R⁵ in the aforementioned formula (1) or (2) is preferably a benzene ring, a naphthalene ring, or an anthracene ring, more preferably a benzene ring or a naphthalene ring, still more preferably a benzene ring, from the viewpoints of ease of synthesis of the compound and availability of starting materials.

Preferred specific embodiments of the second radiation-sensitive composition include the following first and second embodiments.

### First Embodiment

A radiation-sensitive composition containing a polymer having an acid-releasable group and a compound represented by the following formula (1d) (hereinafter may also be referred to as a “compound (CD)”).

### Second Embodiment

A radiation-sensitive composition containing a polymer having a partial structure represented by the following formula (2d) (hereinafter may also be referred to as a “polymer (PD)”).

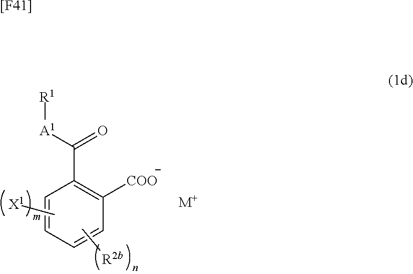

(In the formula (1d), A¹, X¹, R¹, R^{2b}, m, n, and M⁺ have the same meanings as defined in the aforementioned formula (1b).)

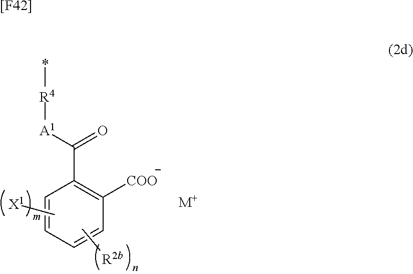

(In the formula (2d), A¹, X¹, R⁴, R^{2b}, m, n, and M⁺ have same meanings as defined in the aforementioned formula (2b).)

Hereinafter, firstly, a radiation-sensitive composition of a first embodiment according to the second radiation-sensitive composition (hereinafter may also be referred to as a “third composition”) will be described. Then, a radiation-sensitive composition of a second embodiment according to the second radiation-sensitive composition (hereinafter may also be referred to as a “fourth composition”) will be described.

### <Third Composition>

The third composition of the present disclosure contains the polymer (A), which is a polymer having an acid-releasable group, and the compound (CD). The polymer (A) contained in the third composition is the same as that contained in the first composition. That is, the same description in relation to the polymer (A) contained in the first composition applies to specific examples, preferred embodiments, structural unit contents, etc. of the polymer (A) contained in the third composition.

### [Compound (CD)]

The compound (CD) is a radiation-sensitive acid-generator that can generate an acid through light exposure. As represented by the aforementioned formula (1d), the compound (CD) is formed of an organic anion and a cation. Through action of radiation, the compound (CD) releases an organic anion via decomposition of the radiation-sensitive cation, and the thus-released organic anion bonds to hydrogen extracted from a component contained in the present composition (e.g., the radiation-sensitive acid-generator itself or a solvent), whereby an acid derived from the organic anion is generated.

The compound (CD) is preferably incorporated into the third composition as an acid diffusion control agent. In other words, in the third composition, the compound (CD) incorporated with the polymer (A) functions as a light-degradable base, to thereby suppress diffusion of a strong acid generated through irradiation with radiation (preferably, an acid derived from a radiation-sensitive acid-generating agent) in the resist film, whereby chemical reaction involving the acid in the light-unexposed part is impeded.

### (Organic Anion)

In the aforementioned formula (1d), when A¹ is —NR³—, specific examples of the monovalent organic group represented by R³ are the same as those described in relation to the aforementioned formula (1b). From the viewpoints of ease of synthesis of the compound (CD) and availability of starting materials, A¹ in the aforementioned formula (1d) is preferably —O— or —S—, more preferably —O—.

Examples of the monovalent organic group represented by R¹ in the aforementioned formula (1d) are the same as those exemplified in relation to the groups represented by R¹ in the aforementioned formula (1b). The monovalent organic group represented by R¹ in the aforementioned formula (1d) is more preferably, among others, a C1 to C20 substituted or unsubstituted monovalent hydrocarbon group, or a C2 to C20 monovalent group formed by inserting —O—, —CO—, or —COO—, NH—, —CONH—, or —S— into a carbon-carbon bond of the substituted or unsubstituted hydrocarbon group. From the viewpoint of further enhancing the sensitivity of the third composition, R¹ in the aforementioned formula (1d) preferably has a polar group. Specifically, similar to the first composition, when R¹ in the aforementioned formula (1d) has a polar group, specific examples of preferred polar groups include a hydroxy group, a carboxy group, a cyano group, a nitro group, an aldehyde group, an acetyl group, a furyl group, and a cyclic acetal group.

To R^{2b} and the parameter n in the aforementioned formula (1d), the same descriptions regarding specific examples and preferred embodiments as those of R^{2b} and the parameter n in the aforementioned formula (1b) apply. Notably, when n is ≥2, and two R^{2b}s in formula (1d) are combined to form a condensed ring structure with a carbon atom to which Ras are bound, the condensed ring structure is preferably a ring structure exhibiting aromaticity. Specific examples of the above case include a naphthalene ring structure or an anthracene ring structure formed with the benzene ring in the formula (1d).

The compound (CD) has a partial structure in which at least one member selected from the group consisting of a chlorine atom, a bromine atom, and an iodine atom (hereinafter may also be referred to as a “particular atom”) is bound to a benzene ring. The particular atom (i.e., X¹ in the aforementioned formula (1d)) is preferably an iodine atom, from the viewpoint of achieving higher sensitivity of the third composition. From the viewpoint of enhancing the sensitivity of the third composition, the number of the particular atoms bound to a benzene ring (i.e., m in the aforementioned formula (1d)) is preferably ≥2, more preferably ≥3, still more preferably 4. When m is ≥2, the species of the particular atoms in a plurality of X¹s may be identical to or different from one another. When m is ≥2, one or more species of the particular atoms in a plurality of X¹s in the aforementioned formula (1d) are preferably an iodine atom. More preferably, all the particular atoms are an iodine atom. No particular limitation is imposed on the bonding position of X¹ with respect to the benzene ring in the aforementioned formula (1d). In other words, the bonding position of X¹ may be any of o-, m-, and p-, with respect to group “—COO⁻”.

From the viewpoint of sensitivity of the third composition, one or more X¹s present in the aforementioned formula (1d) are preferably an iodine atom. Particularly, in a preferred mode of the compound (CD), m in the aforementioned formula (1d) is ≥2, and one or more members of a plurality of X¹s are an iodine atom. In a more preferred mode, m is ≥2, and two or more members of a plurality of X¹s are an iodine atom. In a still preferred mode, m is ≥3, and three or more members of X¹s are an iodine atom. In a particularly preferred mode, m is 4, and all the four X¹s are an iodine atom, from the viewpoint of the sensitivity of the third composition.

### (Cation)

Examples of the cation forming the compound (CD) include the same cations as those exemplified as the cation forming the compound (CB). Similar to the compound (CB), the cation forming the compound (CD) preferably has a sulfonium cation structure or an iodonium cation structure, more preferably a triarylsulfonium cation structure or a diaryliodonium cation structure. Also, from the viewpoint of achieving higher sensitivity of the third composition, M⁺ preferably has a triarylsulfonium cation structure or a diaryliodonium cation structure in which a fluoro group or a fluoroalkyl group (preferably, a fluoromethyl group) is bound to an aromatic ring.

Specific examples of the compound (CD) include compounds represented by the following formulas (d-1) to (d-49). In the following formulas (d-1) to (d-49), M⁺ represents a monovalent cation. Examples of the monovalent cation represented by M⁺ are the same as described above.

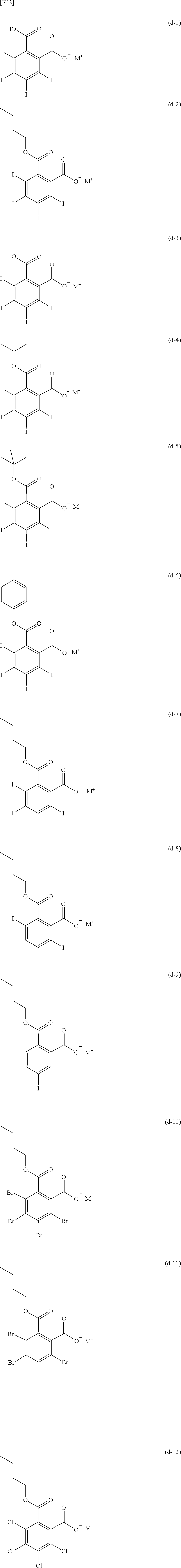

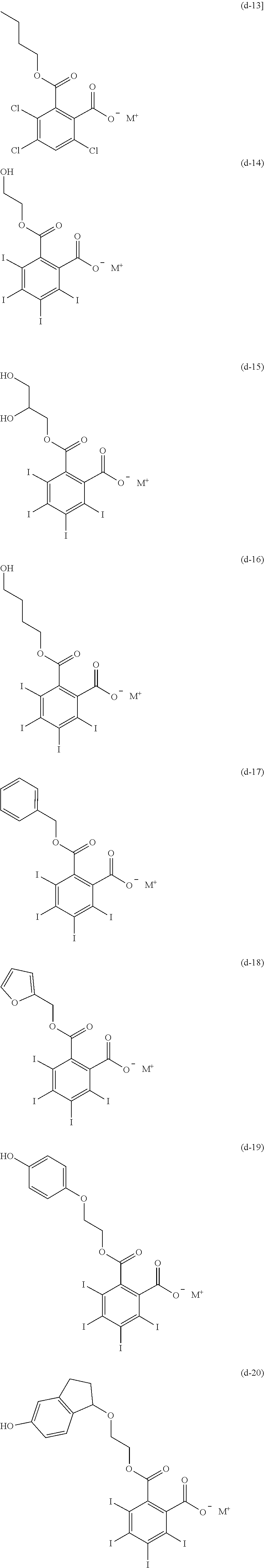

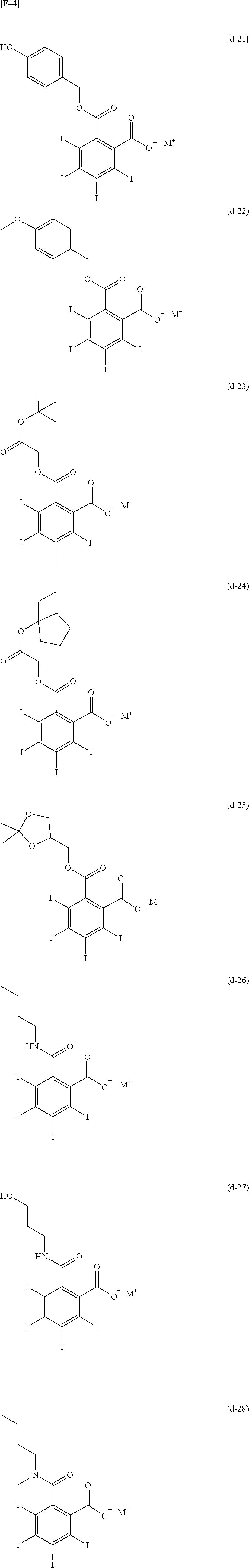

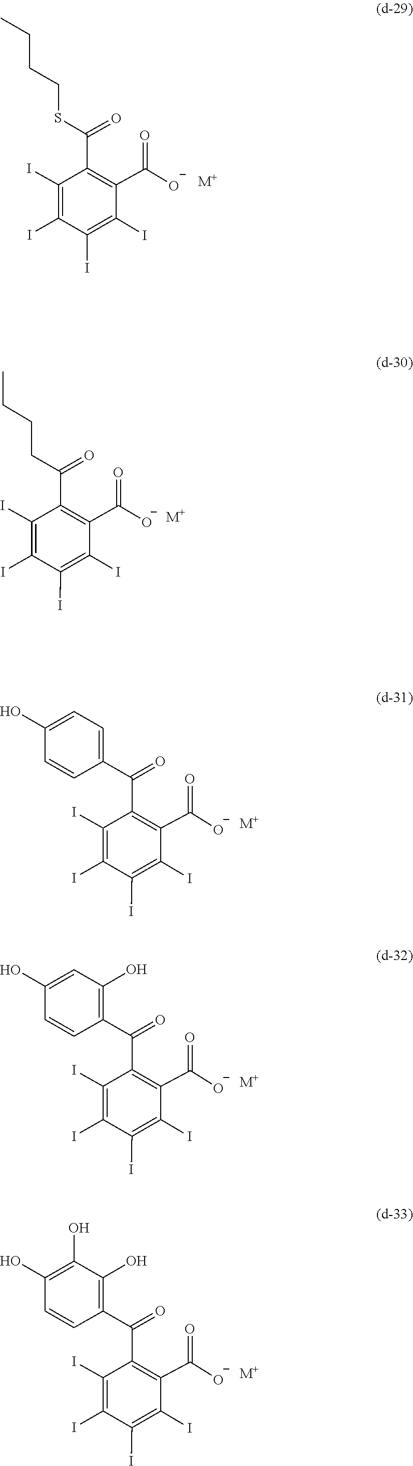

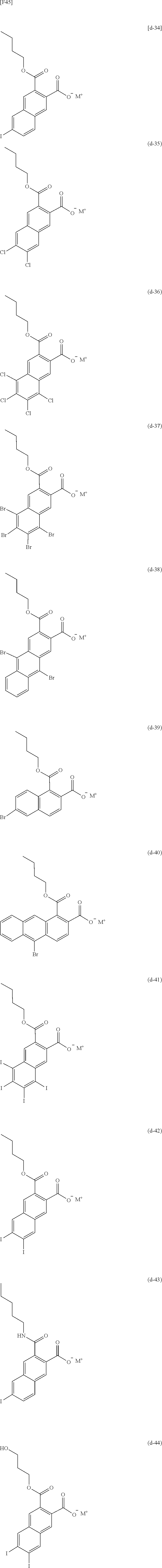

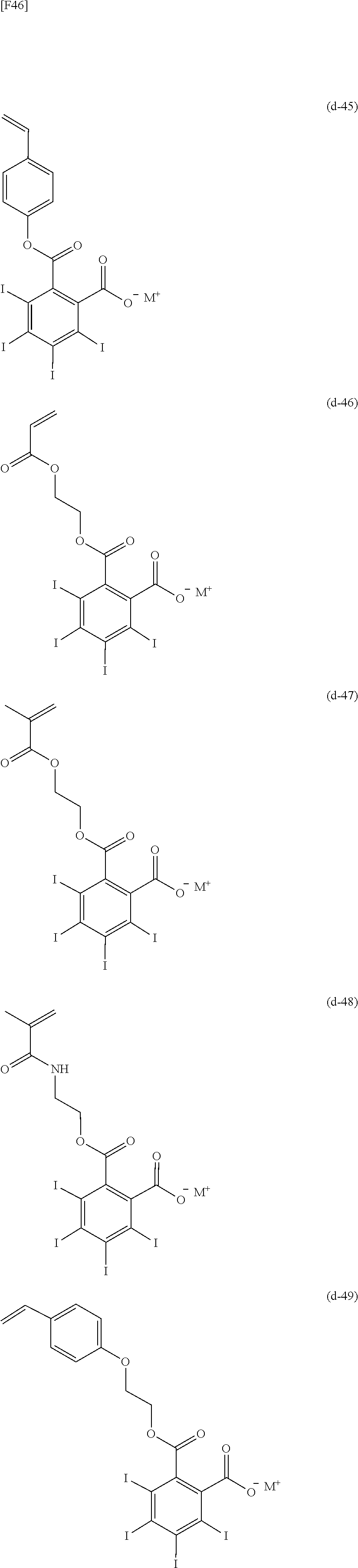

The compound (CD) can be synthesized through customary methods of organic chemistry in appropriate combinations. A specific procedure of the method of synthesizing the compound (CD) will be described, taking the case where A¹ is —O— as an example. According to the following schemes, an carboxylic acid anhydride represented by the following formula (8) is reacted with a compound having a hydroxy group (R¹—OH) in an appropriate solvent in the presence of a base. Subsequently, the thus-formed intermediate is reacted with a sulfonium chloride or the like which can provide an onium cation moiety, to thereby yield the compound (CD). When A¹ is —S—, —NR³—, or a single bond, the same method of synthesizing the compound (CB) applies to synthesis of the aforementioned compound (CD), except that a carboxylic anhydride represented by the following formula (8) is employed. The method of synthesizing the compound (CD) is not limited to the above procedures.

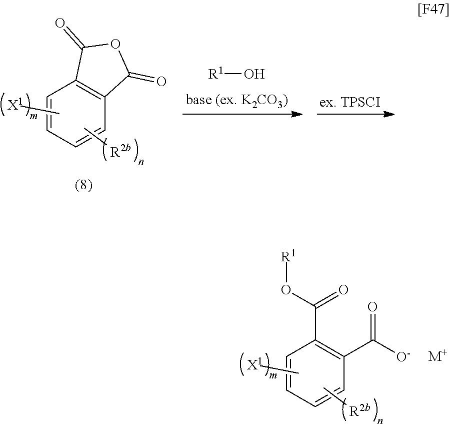

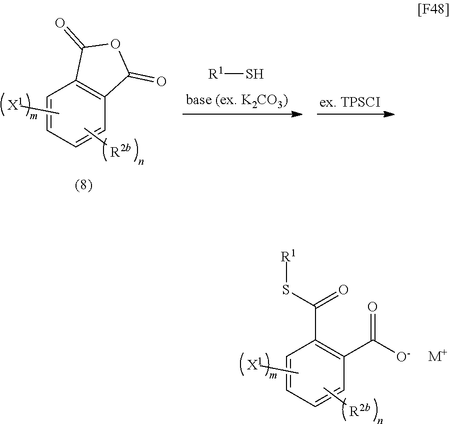

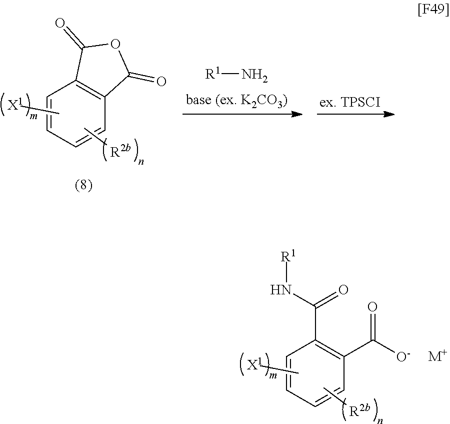

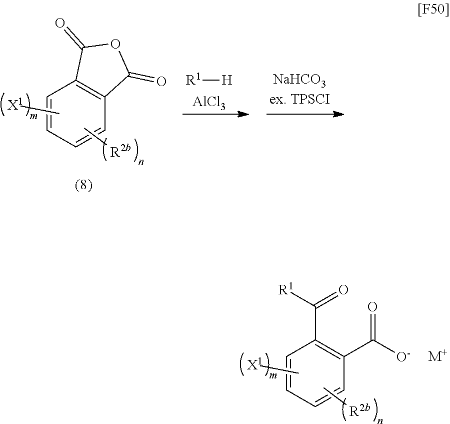

So long as the effects of the present disclosure are not impaired, the third composition may further contain an optional component other than the polymer (A) and the compound (CD). Preferably, the third composition further contains a compound that can generate, upon exposure to light, an acid having an acidity higher than that of a compound represented by the aforementioned formula (1d) (i.e., a radiation-sensitive acid-generating agent). Examples of the radiation-sensitive acid-generating agent incorporated into the third composition include the same as those of the compound (CB) and the additional acid-generating agent as described in relation to the first composition.

When the third composition contains a radiation-sensitive acid-generating agent, the relative amount of the radiation-sensitive acid-generating agent with respect to 100 parts by mass of the polymer (A) is preferably 1 part by mass or more, more preferably 2 parts by mass or more, still more preferably 5 parts by mass or more. Also, the relative amount of the radiation-sensitive acid-generating agent in the third composition with respect to 100 parts by mass of the polymer (A) is preferably 40 parts by mass or less, more preferably 35 parts by mass or less, still more preferably 30 parts by mass or less. Controlling the radiation-sensitive acid-generating agent content to satisfy the above conditions is preferred, since the effects of improving the sensitivity and LWR performance of the third composition and securing a wide process window can be achieved in a well-balanced manner. The radiation-sensitive acid-generating agent incorporated into the third composition may be used singly or in combination of two or more species.

Examples of the optional component incorporated into the third composition include the aforementioned corresponding components, an acid diffusion control agent differing from the compound (CD), the solvent, and the high-fluorine content polymer. Specific examples thereof, preferred embodiments thereof, relative amounts of the components, etc. are the same as those of the first composition.

The third composition may be produced by mixing the polymer (A) and the compound (CD) with an optional component such as solvent at proportions of interest. Regarding the method of producing the third composition, the same descriptions as those of the first composition may be incorporated herein by reference. Also, the preferred range of the solid content of the third composition is the same as that of the first composition.

### <Fourth Composition>

Next, a radiation-sensitive composition of a second embodiment according to the second radiation-sensitive composition (i.e., a fourth composition) will be described. Among the members of the fourth compositions, descriptions of the same members as those of the first composition are omitted.

The fourth composition contains the polymer (PD). The polymer (PD) may serve as a base resin of the fourth composition or a component which serves as a radiation-sensitive acid-generating agent or an acid diffusion control agent and which is incorporated separately with the base resin. In a preferred mode, the fourth composition contains the polymer (PD) as a base resin.

In the aforementioned formula (2d), A¹, X¹, m, n, R^{2b}, and M⁺ are the same as described in relation to the aforementioned formula (1b). Examples of the divalent organic group represented by R⁴ include a group formed by removing any one hydrogen atom from a group exemplified as the monovalent organic group represented by R¹.

The polymer (PD) preferably includes a structural unit having a partial structure represented by the aforementioned formula (2d) (hereinafter may also be referred to as a “structural unit (VII)”). Specific examples of preferred structural units (VII) include those represented by the following formula (2d-1) or (2d-2).

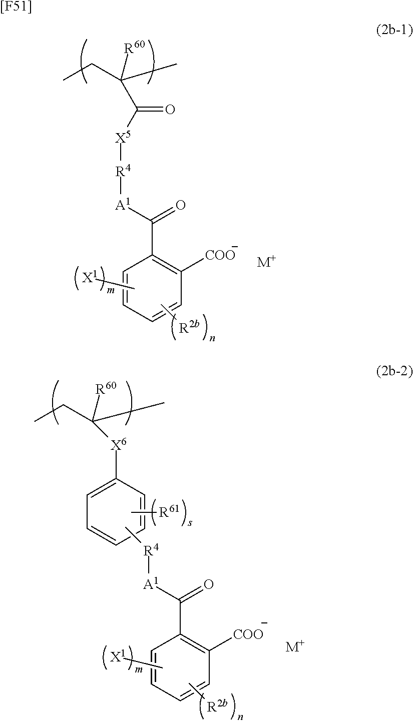

(In the formulas (2d-1) and (2d-2), A¹, X¹, m, n, R^{2b}, R⁴, and M⁺ have the same meanings as those defined in aforementioned formula (2b); and R⁶⁰, X⁵, X⁶, R⁶¹, and s have the same meanings as defined in the aforementioned formulas (2b-1) and (2b-2)).

In the aforementioned formulas (2d-1) and (2d-2), specific examples and preferred embodiments of A¹, X¹, m, n, R^{2b}, and M⁺ are the same as descriptions in relation to the aforementioned formula (1b).

Specific examples and preferred embodiments of R⁴, R⁶¹, and s are the same as descriptions in relation to the aforementioned formulas (2b-1) and (2b-2).

Specific examples of the structural unit (VII) include structural units represented by the aforementioned formulas (v-12) to (v-15), respectively, corresponding to specific examples of the structural unit (V).

In the polymer (PD), the relative amount of structural unit (VII) in all the structural units forming the polymer (PD) is preferably 1 mol % or more, more preferably 3 mol % or more, still more preferably 5 mol % or more. Also, the relative amount of structural unit (VII) in all the structural units forming the polymer (PD) is preferably 50 mol % or less, more preferably 40 mol % or less, still more preferably 30 mol % or less. By adjusting the structural unit (VII) content to satisfy the above conditions, lithographic characteristics such as LWR performance can be enhanced while high sensitivity is maintained, and a wide process window can be secured.

The polymer (PD) may further include a structural unit other than the structural unit (VII). Examples of the additional structural unit include structural units (I) to (IV), described as those in relation to the polymer (A) contained in the first composition, and other structural units. Descriptions of the relative amount of any of the structural units in the polymer (PD) are the same as those in relation to the polymer (A). When the fourth composition contains the polymer (PD) as a base resin, the polymer (PD) preferably has an acid-releasable group, more preferably the structural unit (I). Also, the polymer (PD) may be a polymer which has an acid-releasable group and which includes the structural unit (VII) and a structural unit having an onium salt structure formed of a radiation-sensitive cation and a sulfonic acid anion.

Examples of the optional component incorporated into the fourth composition include the radiation-sensitive acid-generating agent, the acid diffusion control agent, the high-fluorine content polymer, and the solvent, which have been described in relation to the first composition. Specific examples thereof, preferred embodiments thereof, relative amounts of the components, etc. are the same as those of the first composition.

The second radiation-sensitive composition (including the third composition and the fourth composition) prepared through the aforementioned procedure may be used as a positive-type pattern formation composition in formation of a pattern by use of an alkaline developer, or a negative-type pattern formation composition in formation of a pattern by use of a developer containing an organic solvent. The present composition is particularly suitable as a radiation-sensitive composition for EUV light exposure which is used for forming a resist pattern through exposure to extreme UV (EUV) light.

### <<Resist Pattern Formation Method>>

According to the resist pattern formation method of the present disclosure, a resist pattern can be formed by use of the second radiation-sensitive composition. Specifically, a resist pattern can be formed through a method including the application step, light-exposure step, and development step which are described in relation to formation of a resist pattern by use of the first radiation-sensitive composition. Details of the steps are the same as those in the resist pattern formation method by use of the aforementioned first radiation-sensitive composition.

### EXAMPLES

The present disclosure will next be described in detail by way of Synthesis Examples, Examples, and Comparative Examples, which should not be construed as limiting the disclosure to the Examples. Unless otherwise specified, the units “part(s)” and “%” in the Examples are on a mass basis. Measurements were conducted by the following procedures.

### [Weight Average Molecular Weight (Mw) and Number Average Molecular Weight (Mn) of Polymers]

The Mw and Mn of a polymer were determined through gel permeation chromatography (GPC) with GPC columns (G2000HXL×2, G3000HXL×1, and G4000HXL×1) (products of Tosoh Corp.) under the following conditions.
Eluent: tetrahydrofuran (product of Wako Pure Chemical Corporation)
Flow rate: 1.0 mb/min
Sample concentration: 1.0 mass %
Sample injection: 100 μL
Column temperature: 40 °C
Detector: differential refractometer
Standard: monodispersed polystyrene

### 1. Production and Evaluation of Radiation-Sensitive Resin Composition [1]

### <Synthesis of Radiation-Sensitive Acid-Generating Agents>

### [Synthesis Example 1] Synthesis of Acid-Generating Agent (B-1)

To a reaction vessel, a compound represented by the following formula (S-1) (8.2 g), n-butanol (15.0 g), potassium carbonate (2.2 g), and acetonitrile (50 g) were added, and the mixture was stirred at 50 °C for 8 hours. After completion of reaction, the solvent and n-butanol were removed through distillation under reduced pressure, to thereby yield a salt represented by the following formula (S-2). Subsequently, triphenylsulfonium chloride (hereinafter may also be referred to as “TPSCl”) (3.6 g) was added to the salt represented by the following formula (S-2), and a liquid mixture of water and dichloromethane (1:3 (by mass)) was added thereto, to thereby prepare a 0.5M solution. The solution was stirred at room temperature for 1 hour, and then subjected to extraction with dichloromethane, to thereby separate an organic layer. The thus-obtained organic layer was dried over sodium sulfate anhydrate, and the solvent was remove through distillation. The crude product was purified through silica gel chromatography, to thereby yield a radiation-sensitive acid-generating agent represented by the following formula (B-1) (hereinafter may also be referred to as an “acid-generating agent (B-1)”). The scheme of synthesizing the acid-generating agent (B-1) is as follows.

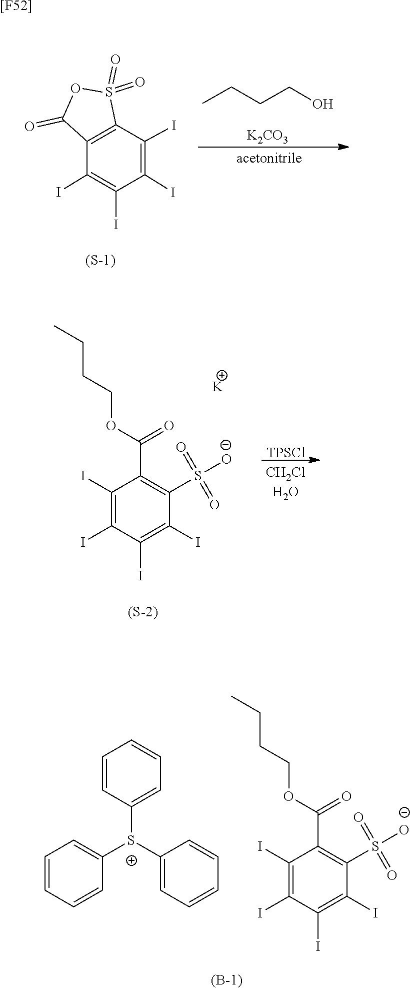

### Synthesis Examples 2 to 7

The procedure of Synthesis Example 1 was repeated with an appropriate choice of starting materials, to thereby synthesize radiation-sensitive acid-generating agents represented by the following formulas (B-2) to (B-6) and (BB-1), respectively (hereinafter may also be referred to as “acid-generating agents (B-2) to (B-6), and (BB-1)”).

### Synthesis Examples 8 and 9

Through a known method, a radiation-sensitive acid-generating agent represented by the following formula (BB-2) or (BB-3) (hereinafter may also be referred to as a “acid-generating agent (BB-2) or (BB-3)”) was synthesized.

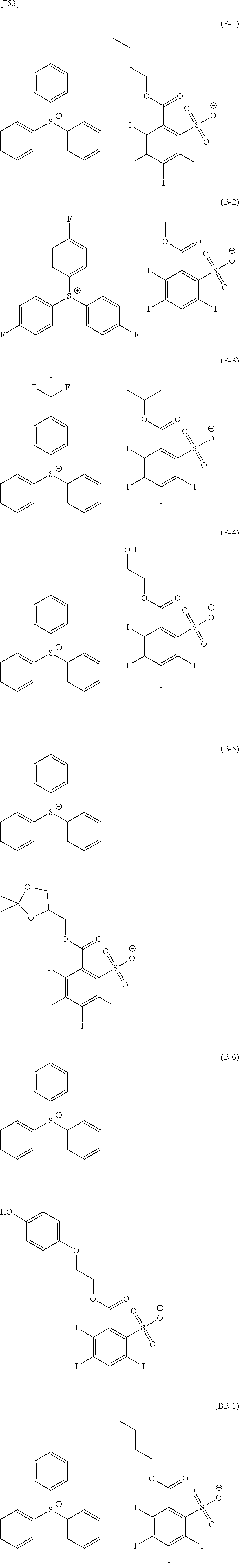

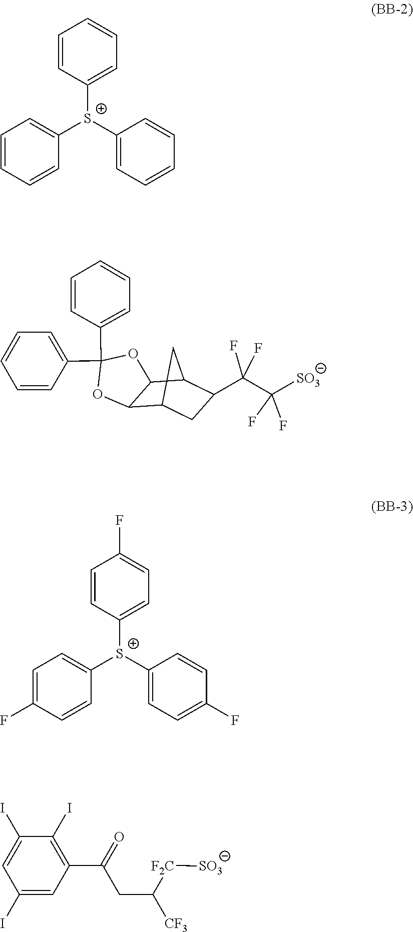

### <Synthesis of Monomers>

### Synthesis Examples 10 to 12

The procedure of Synthesis Example 1 was repeated with an appropriate choice of starting materials, to thereby synthesize a monomer represented by the following formula (M-12) or (M-13) (hereinafter may also be referred to as a “compound (M-12) or (M-13)”). Also, through a known method, a monomer represented by the following formula (M-14) (hereinafter may also be referred to as a “compound (M-14)”) was synthesized.

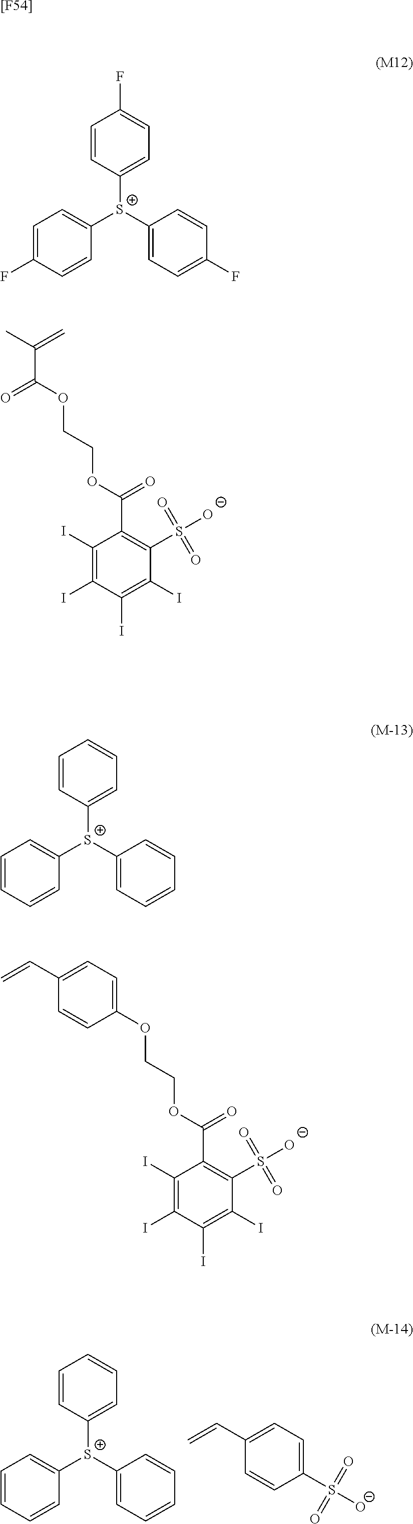

### <Synthesis of Polymers>

Monomers employed in synthesis of respective polymers (hereinafter may also be referred to as “compounds (M-1) to (M-15)”) are as follows. The compounds (M-12) to (M-14) were synthesized in the above Synthesis Example.

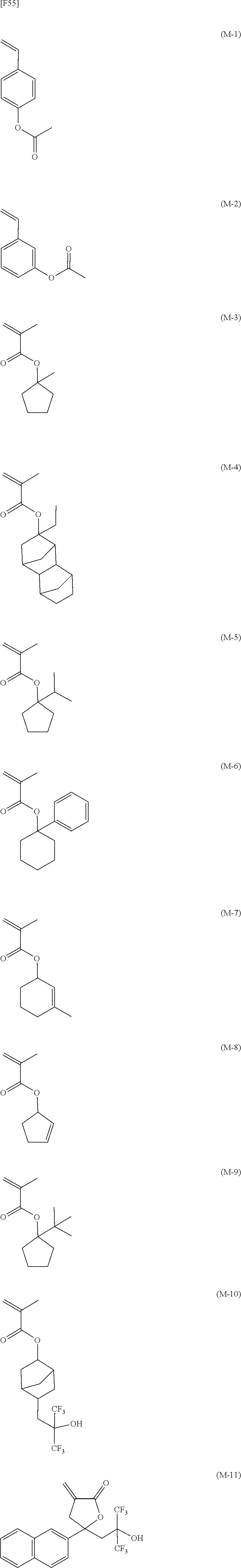

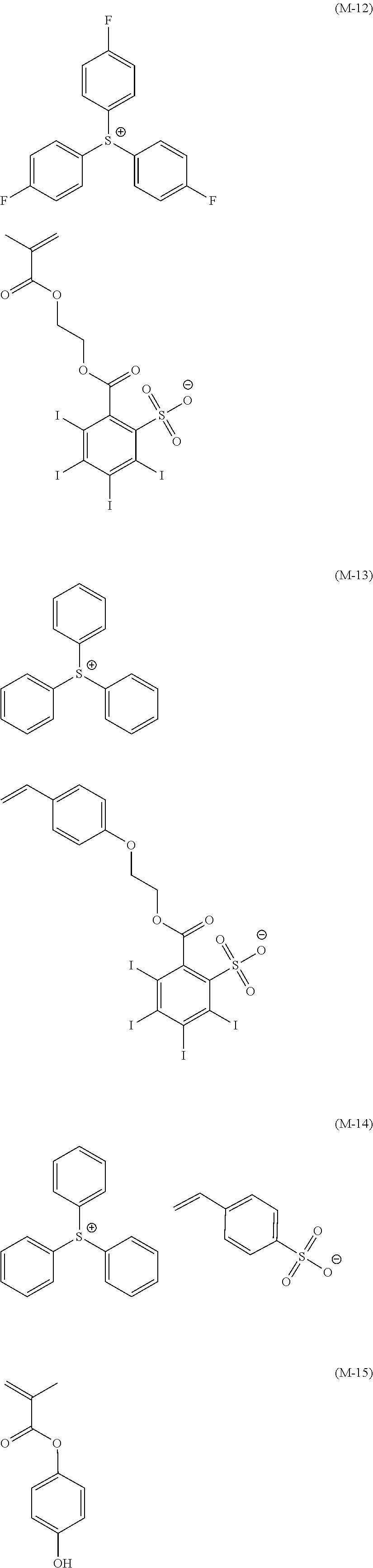

### [Polymerization Example 1] Synthesis of Polymer (A-1)

The compound (M-1) and the compound (M-3) were dissolved in 1-methoxy-2-propanol so as to adjust the mole ratio to 40/60 (200 parts by mass with respect to all the monomers). Subsequently, azobis(isobutyronitrile) serving as a polymerization initiator was added at 6 mol % (with respect to all the monomers), to thereby prepare a monomer solution. Separately, 1-methoxy-2-propanol was added to a vacant reaction vessel (100 parts by mass with respect to all the monomers), and the mixture was heated at 85 °C under stirring. The above-prepared monomer solution was added dropwise thereto over 3 hours. Thereafter, the reaction was further heated at 85 °C for 3 hours. After completion of polymerization reaction, the polymerization solution was cooled to room temperature. The thus-cooled polymerization solution was put into hexane (500 parts by mass with respect to polymerization solution), and precipitated white powder was separated through filtration. The thus-separated white powder was washed twice with hexane (100 parts by mass with respect to polymerization solution) and separated through filtration. The separated powder was dissolved in 1-methoxy-2-propanol (300 parts by mass). To the resultant solution, methanol (500 parts by mass), triethylamine (50 parts by mass), and ultrapure water (10 parts by mass) were added, and the mixture was subjected to hydrolysis reaction at 70 °C for 6 hours under stirring.

After completion of reaction, the solvent was removed through distillation, and the resultant solid was dissolved in acetone (100 parts by mass). The solution was added dropwise to water (500 parts by mass), to thereby solidify resin, and the thus-obtained solid was separated through filtration. The solid was dried at 50 °C for 12 hours, to thereby yield a polymer (A-1) in the form of white powder.

### Polymerization Examples 2 to 12

The procedure of Polymerization Example 1 was repeated, except that types and proportions of the monomers were changed to those shown in Table 1, to thereby synthesize polymers (A-2) to (A-12). Notably, the amount of monomer shown in Table 1 refers to an amount (mol %) of each monomer used in synthesis of the relevant polymer, with respect to the total amount of the monomers used.

### Polymerization Example 13

The compound (M-15) and the compound (M-3) were used so as to adjust the mole ratio to 40/60, and polymerization reaction was performed through a known method, to thereby synthesize a polymer (A-13).

TABLE 1

| | | Monomer 1 | | Monomer 2 | | Monomer 3 | | | |
|---|---|---|---|---|---|---|---|---|---|
| | Polymer | | Amount | | Amount | | Amount | | |
| | name | Type | (mol %) | Type | (mol %) | Type | (mol %) | Mw | Mw/Mn |
| Polymerization | A-1 | M-1 | 40 | M-3 | 60 | — | — | 5700 | 1.6 |
| Example 1 | | | | | | | | | |
| Polymerization | A-2 | M-1 | 40 | M-4 | 60 | — | — | 5800 | 1.6 |
| Example 2 | | | | | | | | | |
| Polymerization | A-3 | M-1/M-2 | 30/10 | M-5 | 60 | — | — | 6100 | 1.7 |
| Example 3 | | | | | | | | | |
| Polymerization | A-4 | M-1 | 40 | M-6 | 60 | — | — | 6200 | 1.5 |
| Example 4 | | | | | | | | | |
| Polymerization | A-5 | M-1 | 40 | M-7 | 60 | — | — | 5500 | 1.5 |
| Example 5 | | | | | | | | | |
| Polymerization | A-6 | M-1 | 40 | M-8 | 60 | — | — | 5400 | 1.5 |
| Example 6 | | | | | | | | | |
| Polymerization | A-7 | M-1 | 40 | M-9 | 60 | — | — | 6000 | 1.7 |
| Example 7 | | | | | | | | | |
| Polymerization | A-8 | M-1 | 30 | M-3 | 60 | M-10 | 10 | 6900 | 1.7 |
| Example 8 | | | | | | | | | |
| Polymerization | A-9 | M-1 | 30 | M-3 | 60 | M-11 | 10 | 6800 | 1.7 |
| Example 9 | | | | | | | | | |
| Polymerization | A-10 | M-1 | 30 | M-3 | 60 | M-12 | 10 | 6700 | 1.6 |
| Example 10 | | | | | | | | | |
| Polymerization | A-11 | M-1 | 30 | M-3 | 60 | M-13 | 10 | 6900 | 1.6 |
| Example 11 | | | | | | | | | |
| Polymerization | A-12 | M-1 | 30 | M-3 | 60 | M-14 | 10 | 6800 | 1.6 |
| Example 12 | | | | | | | | | |
| Polymerization | A-13 | M-15 | 40 | M-3 | 60 | — | — | 7000 | 1.7 |
| Example 13 | | | | | | | | | |

### <Production of Radiation-Sensitive Resin Compositions>

The polymers [A], radiation-sensitive acid-generating agents [B], acid diffusion control agents [D], and organic solvents [E] employed in production of the radiation-sensitive resin compositions are as follows,

### [A] Polymers

A-1 to A-13: polymers (A-1) to (A-13) synthesized in the aforementioned Polymerization Examples 1 to 13.

### [B] Radiation-Sensitive Acid-Generating Agents

B-1 to B-6: acid-generating agents (B-1) to (B-6) synthesized in the aforementioned Polymerization Examples 1 to 6.

BB-1 to BB-3: acid-generating agents (BB-1) to (BB-3) synthesized in the aforementioned Polymerization Examples 7 to 9.

### [D] Acid Diffusion Control Agents

DD-1 to DD-3: compounds represented by the following formulas (DD-1) to (DD-3), respectively.

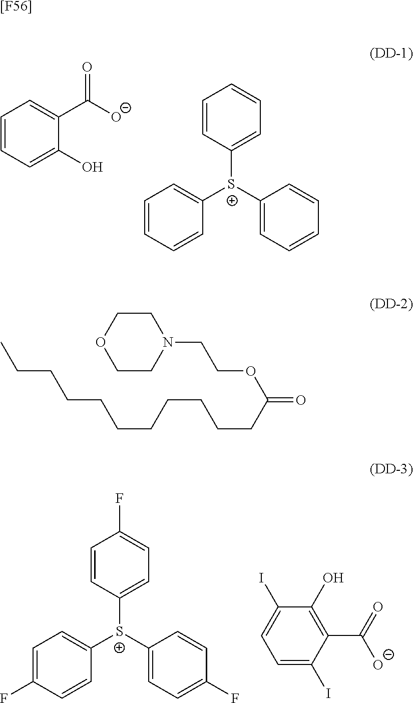

### [E] Organic Solvents

E-1: Propylene glycol acetate monomethyl ether
E-2: Propylene glycol-1-monomethyl ether

### Example 1

There were mixed the polymer (A-1) (100 parts by mass), the acid-generating agent (B-1) (20 parts by mass), the acid diffusion control agent (DD-1) (20 mol % with respect to the acid-generating agent (B-1)), the organic solvent (E-1) (4, 800 parts by mass), and the organic solvent (E-2) (2,000 parts by mass), and the resultant liquid mixture was filtered through a membrane filter (pore size: 0.20 μm), to thereby prepare a radiation-sensitive resin composition (R-1).

### Examples 2 to 20 and Comparative Examples 1 to 2

The procedure of Example 1 was repeated, except that components whose types and amounts are shown in Table 2 were used, to thereby prepare radiation-sensitive resin compositions (R-2) to (R-20), (CR-1), and (CR-2). Notably, in Table 2, the amount of acid diffusion control agent [D] is a ratio (mol %) with respect to the amount of radiation-sensitive acid-generating agent [B] (total amount in the case two agents were used).

TABLE 2

| | | | | [B] Radiation- | | | | | |
|---|---|---|---|---|---|---|---|---|---|
| | | | | sensitive acid- | | | | | |
| | Radiation- | [A] Polymer | | generating agent | | [D] Acid diffusion | | [E] Organic solvent | |
| | sensitive | | Amount | | Amount | control agent | | | Amount |
| | resin | | (part(s) | | (part(s) | | Amount | | (part(s) |
| | composition | Type | by mass) | Type | by mass) | Type | (mol %) | Type | by mass) |
| Example 1 | R-1 | A-1 | 100 | B-1 | 20 | DD-1 | 20 | E-1/E-2 | 4800/2000 |
| Example 2 | R-2 | A-1 | 100 | B-2 | 20 | DD-1 | 20 | E-1/E-2 | 4800/2000 |
| Example 3 | R-3 | A-1 | 100 | B-3 | 20 | DD-1 | 20 | E-1/E-2 | 4800/2000 |
| Example 4 | R-4 | A-1 | 100 | B-4 | 20 | DD-1 | 20 | E-1/E-2 | 4800/2000 |
| Example 5 | R-5 | A-1 | 100 | B-5 | 20 | DD-1 | 20 | E-1/E-2 | 4800/2000 |
| Example 6 | R-6 | A-1 | 100 | B-6 | 20 | DD-1 | 20 | E-1/E-2 | 4800/2000 |
| Example 7 | R-7 | A-2 | 100 | B-2 | 20 | DD-1 | 20 | E-1/E-2 | 4800/2000 |
| Example 8 | R-8 | A-3 | 100 | B-2 | 20 | DD-1 | 20 | E-1/E-2 | 4800/2000 |
| Example 9 | R-9 | A-4 | 100 | B-2 | 20 | DD-1 | 20 | E-1/E-2 | 4800/2000 |
| Example 10 | R-10 | A-5 | 100 | B-2 | 20 | DD-1 | 20 | E-1/E-2 | 4800/2000 |
| Example 11 | R-11 | A-6 | 100 | B-2 | 20 | DD-1 | 20 | E-1/E-2 | 4800/2000 |
| Example 12 | R-12 | A-7 | 100 | B-2 | 20 | DD-2 | 20 | E-1/E-2 | 4800/2000 |
| Example 13 | R-13 | A-8 | 100 | B-2 | 20 | DD-1 | 20 | E-1/E-2 | 4800/2000 |
| Example 14 | R-14 | A-9 | 100 | B-2 | 20 | DD-1 | 20 | E-1/E-2 | 4800/2000 |
| Example 15 | R-15 | A-1 | 100 | B-2 | 20 | DD-3 | 20 | E-1/E-2 | 4800/2000 |
| Example 16 | R-16 | A-10 | 100 | B-1 | 20 | DD-1 | 20 | E-1/E-2 | 4800/2000 |
| Example 17 | R-17 | A-11 | 100 | B-1 | 20 | DD-1 | 20 | E-1/E-2 | 4800/2000 |
| Example 18 | R-18 | A-13 | 100 | B-2 | 20 | DD-1 | 20 | E-1/E-2 | 4800/2000 |
| Example 19 | R-19 | A-1 | 100 | B-1/BB-2 | 15/5 | DD-1 | 20 | E-1/E-2 | 4800/2000 |
| Example 20 | R-20 | A-1 | 100 | B-1/BB-3 | 15/5 | DD-1 | 20 | E-1/E-2 | 4800/2000 |
| Comparative | CR-1 | A-1 | 100 | BB-1 | 20 | DD-1 | 20 | E-1/E-2 | 4800/2000 |
| Example 1 | | | | | | | | | |
| Comparative | CR-2 | A-12 | 100 | BB-1 | 20 | DD-1 | 20 | E-1/E-2 | 4800/2000 |
| Example 2 | | | | | | | | | |

### <Formation of Resist Pattern>

By use of each of the prepared radiation-sensitive resin compositions, a resist pattern was formed through the following procedure.

A 12-inch silicon wafer having an under-layer film having a thickness of 20 nm (AL-412, product of Brewer Science, Inc.) was used. Onto the surface of the wafer, each of the above-prepared radiation-sensitive resin compositions was applied by means of a spin coater (CLEAN TRACK ACT12, product of Tokyo Electron Ltd.). The wafer was subjected to pre-baking (PB) at 130 °C for 60 seconds and then cooled at 23 °C for 30 seconds, to thereby form a resist film having a mean thickness of 50 nm. Subsequently, the resist film was irradiated with EUV light by means of an EUV exposure device (NXE3300, product of ASML, NA=0.33, lighting condition: Conventional s=0.89, and mask: imecDEFECT32FFR02). After the EUV light exposure, the resist film was subjected to PEB (post exposure baking) at 110 °C for 60 seconds. Then, development was performed by use of 2.38 mass % aqueous TMAH at 23 °C for 30 seconds, to thereby form a positive-type line-and-space pattern (32 nm).

### <Evaluation>

Each of the formed resist patterns was evaluated in terms of sensitivity, LWR performance, and process window of the radiation-sensitive resin composition, through measurement according to the following methods. Dimensions of the resist pattern were measured under a scanning electron microscope (CG-4100, product of Hitachi High Technology Co., Ltd.). Table 3 shows the results of evaluation.

### [Sensitivity]

In the aforementioned “Formation of resist pattern”, a dose which can form a line-and-space pattern having a width of 32 nm was employed as an optimum dose, and the optimum dose was represented by Eop (unit: mJ/cm²). The lower the value of Eop, the better the sensitivity. The sensitivity was evaluated as “A”, when Eop was 30 mJ/cm² or lower. The sensitivity was evaluated as “B”, when Eop was higher than 30 mJ/cm² and 32 mJ/cm² or lower. The sensitivity was evaluated as “C”, when Eop was higher than 32 mJ/cm² was evaluated as “C”.

### [LWR Performance]

Each of the above-formed resist patterns was observed from above under the aforementioned scanning electron microscope, and line width was measured at 50 points selected at random. Variation in width (30) was determined to serve as LWR performance (unit: nm). The smaller the variation in width (30), the smaller the variation in line width (i.e., the more excellent the LWR performance). LWR performance was evaluated as “A”, when the variation in width (30) was 4.0 nm or less. LWR performance was evaluated as “B”, when the variation in width (30) was more than 4.0 nm and 4.2 nm or less. LWR performance was evaluated as “C”, when the variation in width (30) was more than 4.2 nm.

### [Process Window]

The term “process window” refers to a range of a resist dimension parameter which ensures formation of a pattern having no bridge defects and collapses. By use of a mask for forming a 32-nm line-and-space pattern (1L/1S), various patterns were formed corresponding to exposure doses ranging from a low value to a high value. Generally, application of light of a lower exposure dose results in undesired defects such as bridge formation between patterns, and application of light of a higher exposure dose results in defects such as collapsing of patterns. The difference between the maximum and minimum values of the resist dimension parameter in which the above defects were not observed was employed as a “critical dimension (CD) margin” (unit: nm). The greater the CD margin, the wider (more excellent) the process window. In the case where the CD margin was 30 nm or more, the score was “A”. In the case where the CD margin was 28 nm or more and less than 30 nm, the score was “B”. In the case where the CD margin was less than 28 nm, the score was “C”.

TABLE 3

| | Radiation- | | | |
|---|---|---|---|---|
| | sensitive resin | | LWR | Process |
| | composition | Sensitivity | performance | window |
| Example 1 | R-1 | B | B | B |
| Example 2 | R-2 | A | B | B |
| Example 3 | R-3 | A | B | B |
| Example 4 | R-4 | A | B | B |
| Example 5 | R-5 | A | B | B |
| Example 6 | R-6 | A | B | B |
| Example 7 | R-7 | A | B | B |
| Example 8 | R-8 | A | B | B |
| Example 9 | R-9 | A | B | B |
| Example 10 | R-10 | A | B | B |
| Example 11 | R-11 | A | B | B |
| Example 12 | R-12 | A | B | B |
| Example 13 | R-13 | A | B | B |
| Example 14 | R-14 | A | B | B |
| Example 15 | R-15 | A | A | B |
| Example 16 | R-16 | B | A | A |
| Example 17 | R-17 | B | A | A |
| Example 18 | R-18 | A | B | B |
| Example 19 | R-19 | B | B | B |
| Example 20 | R-20 | A | B | B |
| Comparative | CR-1 | C | C | C |
| Example 1 | | | | |
| Comparative | CR-2 | C | B | C |
| Example 2 | | | | |

As shown in Table 3, the radiation-sensitive resin compositions of Examples 1 to 20 were found to exhibit high sensitivity, sufficiently wide CD margin, and excellent LWR performance. Among these compositions, compositions were evaluated with a score of sensitivity “A” in the following cases; cases in which the acid-generating agents (B-2) and (B-3) each formed of an anion moiety having a structure corresponding to the aforementioned formula (1b) and a cation moiety having a fluorine atom were used as the compound (CB) (Examples 2, 3, 7 to 15, and 18); cases in which the acid-generating agents (B-4) to (B-6) each formed of an anion moiety having a polar group were used as the compound (CB) (Examples 4 to 6); and a case in which, among two radiation-sensitive acid-generating agents [B], the acid-generating agent (BB-3) formed of an anion moiety having an iodine atom was used as an additional acid-generating agent (Example 20). Also, in the cases in which the onium salt (DD-3) formed of an anion moiety having an iodine atom was used as the acid diffusion control agent [D] (Example 15), and in which the polymers (A-10) and (A-11) each including a structural unit having an iodo group as the polymers [A] were used (Examples 16 and 17), an excellent LWR performance with a score “A” was provided, while high sensitivity was maintained. In addition, in Examples 16 and 17, the CD margin was evaluated as a score “A”, which is a suitable result.

In contrast, the radiation-sensitive resin composition of Comparative Example 1 exhibited a score of “C” in all measured properties. The radiation-sensitive resin composition of Comparative Example 2 exhibited a sensitivity score and a process window score of “C”, which were inferior to those of Examples 1 to 20.

### 2. Production and Evaluation of Radiation-Sensitive Resin Composition [2]

### <Synthesis of Acid Diffusion Control Agent>

### [Synthesis Example 13] Synthesis of Acid Diffusion Control Agent (D-1)

To a reaction vessel, a compound represented by the following formula (T-1) (5.0 g), n-butanol (11.4 g), potassium carbonate (1.2 g), and acetonitrile (20 g) were added, and the mixture was stirred at 50 °C for 10 hours. After completion of reaction, the solvent and n-butanol were removed through distillation, to thereby yield a salt represented by the following formula (T-2). Subsequently, TPSCl (2.3 g) was added to the salt represented by the following formula (T-2), and a liquid mixture of water and dichloromethane (1:3 (by mass)) was added thereto, to thereby prepare a 0.5M solution. The solution was stirred at room temperature for 1 hour, and then subjected extraction with dichloromethane, to thereby separate an organic layer. The thus-obtained organic layer was dried over sodium sulfate anhydrate, and the solvent was removed through distillation. Thus, an acid diffusion control agent represented by the following formula (D-1) (hereinafter may also be referred to as “acid diffusion control agent (D-1)”) was yielded. The scheme of synthesizing the acid diffusion control agent (D-1) is as follows.

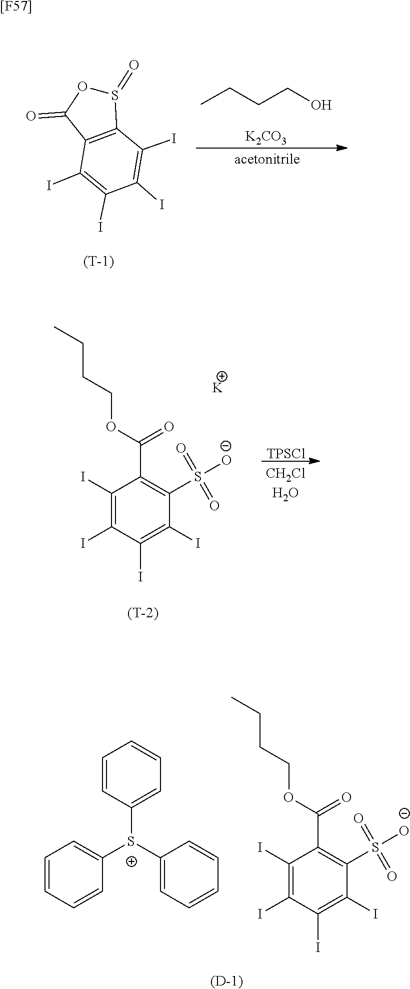

### Synthesis Examples 14 to 20

The procedure of Synthesis Example 13 was repeated with an appropriate choice of starting materials, to thereby synthesize acid diffusion control agents represented by the following formulas (D-2) to (D-7) and (DD-4), respectively (hereinafter may also be referred to as “acid diffusion control agents (D-2) to (D-7) and (DD-4)”).

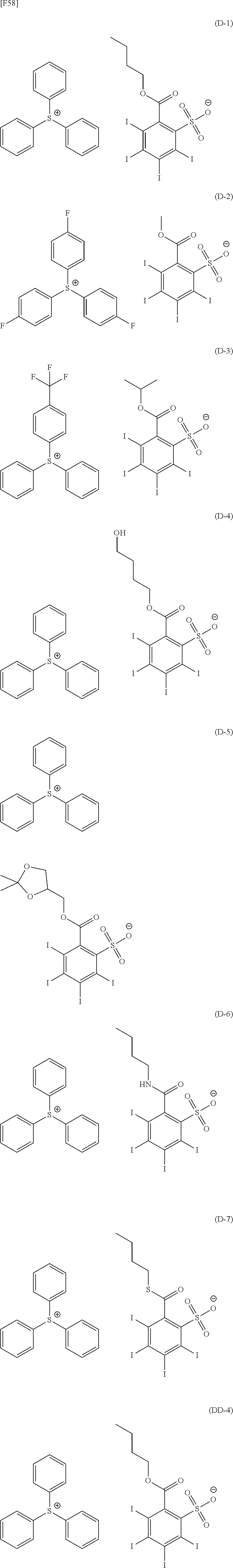

### <Synthesis of Monomers>

### Synthesis Examples 21 and 22

The procedure of Synthesis Example 1 was repeated with an appropriate choice of starting materials, to thereby synthesize a monomer represented by the following formula (M-17) or (M-18) (hereinafter may also be referred to as a “compound (M-17) or (M-18)”).

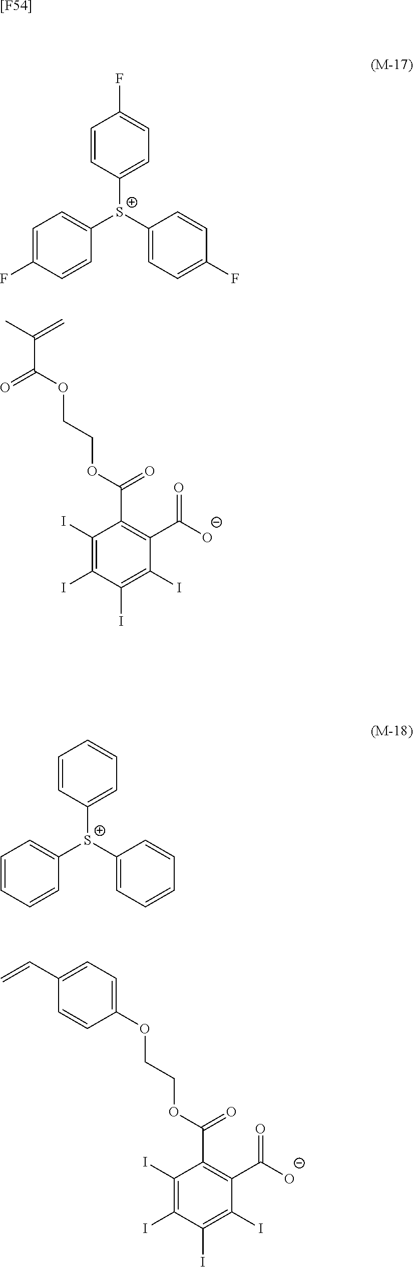

### <Synthesis of Polymers>

### Polymerization Example 14

In Polymerization Example 14, the compound (M-1) employed in the aforementioned “1. Production and evaluation of radiation-sensitive resin composition [1]” and a compound represented by the following formula (M-16) were used as monomers. Also, the procedure of the Polymerization Example 1 included in the aforementioned “1. Production and evaluation of radiation-sensitive resin composition [1]” was repeated, except that types and proportions of the monomers were changed to those shown in Table 4, to thereby synthesize a polymer (A-14). Notably, the amount of monomer shown in Table 4 refers to an amount (mol %) of each monomer used in synthesis of the polymer (A-14), with respect to the total amount of the monomers used.

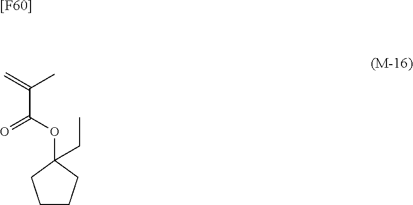

### Polymerization Examples 15 to 17

The procedure of the Polymerization Example 1 included in the aforementioned “1. Production and evaluation of radiation-sensitive resin composition [1]” was repeated, except that types and proportions of the monomers were changed to those shown in Table 4, to thereby synthesize polymers (A-15) to (A-17). Notably, the amount of monomer shown in Table 4 refers to an amount (mol %) of each monomer used in synthesis of the relevant polymer, with respect to the total amount of the monomers used.

TABLE 4

| | | Monomer 1 | | Monomer 2 | | Monomer 3 | | Monomer 4 | | | |
|---|---|---|---|---|---|---|---|---|---|---|---|
| | Polymer | | Amount | | Amount | | Amount | | Amount | | |
| | name | Type | (mol %) | Type | (mol %) | Type | (mol %) | Type | (mol %) | Mw | Mw/Mn |
| Polymerization | A-14 | M-1 | 40 | M-16 | 60 | — | — | — | — | 5800 | 1.6 |
| Example 14 | | | | | | | | | | | |
| Polymerization | A-15 | M-1 | 35 | M-16 | 55 | M-17 | 10 | — | — | 6100 | 1.7 |
| Example 15 | | | | | | | | | | | |
| Polymerization | A-16 | M-1 | 35 | M-16 | 55 | M-18 | 10 | — | — | 6000 | 1.7 |
| Example 16 | | | | | | | | | | | |
| Polymerization | A-17 | M-1 | 30 | M-16 | 50 | M-17 | 5 | M-14 | 15 | 6100 | 1.7 |
| Example 17 | | | | | | | | | | | |

### <Production of Radiation-Sensitive Resin Compositions>

The polymers [A], radiation-sensitive acid-generating agents [B], acid diffusion control agents [D], and organic solvents [E] employed in production of the radiation-sensitive resin compositions are as follows. Notably, regarding the following polymers [A], radiation-sensitive acid-generating agents [B], acid diffusion control agents [D], and organic solvents [E], the same compounds as those employed in the aforementioned “1. Production and evaluation of radiation-sensitive resin composition [1]” are denoted by the same abbreviations.

### Example 21

There were mixed the polymer (A-1) (100 parts by mass), the acid-generating agent (BB-2) (20 parts by mass), the acid diffusion control agent (D-1) (20 mol % with respect to the acid-generating agent (BB-2)), the organic solvent (E-1) (4,800 parts by mass), and the organic solvent (E-2) (2,000 parts by mass), and the resultant liquid mixture was filtered through a membrane filter (pore size: 0.20 μm), to thereby prepare a radiation-sensitive resin composition (R-21).

### Examples 22 to 39 and Comparative Example 3

The procedure of Example 21 was repeated, except that components whose types and amounts are shown in Table 5 were used, to thereby prepare radiation-sensitive resin compositions (R-22) to (R-39) and (CR-3). Notably, in Table 5, the amount of acid diffusion control agent [D] is a ratio (mol %) with respect to the amount of radiation-sensitive acid-generating agent [B].

TABLE 5

| | | | | [B] Radiation- | | | | | |
|---|---|---|---|---|---|---|---|---|---|
| | | | | sensitive acid- | | | | | |
| | Radiation- | [A] Polymer | | generating agent | | [D] Acid diffusion | | [E] Organic solvent | |
| | sensitive | | Amount | | Amount | control agent | | | Amount |
| | resin | | (part(s) | | (part(s) | | Amount | | (part(s) |
| | composition | Type | by mass) | Type | by mass) | Type | (mol %) | Type | by mass) |
| Example 21 | R-21 | A-1 | 100 | BB-2 | 20 | D-1 | 20 | E-1/E-2 | 4800/2000 |
| Example 22 | R-22 | A-1 | 100 | BB-2 | 20 | D-2 | 20 | E-1/E-2 | 4800/2000 |
| Example 23 | R-23 | A-1 | 100 | BB-2 | 20 | D-3 | 20 | E-1/E-2 | 4800/2000 |
| Example 24 | R-24 | A-1 | 100 | BB-2 | 20 | D-4 | 20 | E-1/E-2 | 4800/2000 |
| Example 25 | R-25 | A-1 | 100 | BB-2 | 20 | D-5 | 20 | E-1/E-2 | 4800/2000 |
| Example 26 | R-26 | A-1 | 100 | BB-2 | 20 | D-6 | 20 | E-1/E-2 | 4800/2000 |
| Example 27 | R-27 | A-1 | 100 | BB-2 | 20 | D-7 | 20 | E-1/E-2 | 4800/2000 |
| Example 28 | R-28 | A-14 | 100 | BB-2 | 20 | D-2 | 20 | E-1/E-2 | 4800/2000 |
| Example 29 | R-29 | A-3 | 100 | BB-2 | 20 | D-2 | 20 | E-1/E-2 | 4800/2000 |
| Example 30 | R-30 | A-4 | 100 | BB-2 | 20 | D-2 | 20 | E-1/E-2 | 4800/2000 |
| Example 31 | R-31 | A-5 | 100 | BB-2 | 20 | D-2 | 20 | E-1/E-2 | 4800/2000 |
| Example 32 | R-32 | A-6 | 100 | BB-2 | 20 | D-2 | 20 | E-1/E-2 | 4800/2000 |
| Example 33 | R-33 | A-7 | 100 | BB-2 | 20 | D-2 | 20 | E-1/E-2 | 4800/2000 |
| Example 34 | R-34 | A-8 | 100 | BB-2 | 20 | D-2 | 20 | E-1/E-2 | 4800/2000 |
| Example 35 | R-35 | A-9 | 100 | BB-2 | 20 | D-2 | 20 | E-1/E-2 | 4800/2000 |
| Example 36 | R-36 | A-10 | 100 | BB-2 | 20 | D-2 | 20 | E-1/E-2 | 4800/2000 |
| Example 37 | R-37 | A-15 | 100 | BB-2 | 20 | D-2 | 20 | E-1/E-2 | 4800/2000 |
| Example 38 | R-38 | A-16 | 100 | BB-2 | 20 | D-2 | 20 | E-1/E-2 | 4800/2000 |
| Example 39 | R-39 | A-17 | 100 | — | — | — | — | E-1/E-2 | 4800/2000 |
| Comparative | CR-3 | A-1 | 100 | BB-2 | 20 | DD-4 | 20 | E-1/E-2 | 4800/2000 |
| Example 3 | | | | | | | | | |

### <Formation of Resist Pattern>

The procedure of “1. Production and evaluation of radiation-sensitive resin composition [1]” was repeated, by use of each of the above-prepared radiation-sensitive resin compositions (R-21) to (R-39) and (CR-3), to thereby form a positive-type 32-nm line-and-space pattern.

### <Evaluation>

In a manner similar to that of “1. Production and evaluation of radiation-sensitive resin composition [1]”, each of the above-formed resist patterns was evaluated in terms of sensitivity, LWR performance, and process window of the radiation-sensitive resin composition. Table 6 shows the results of evaluation.

TABLE 6

| | Radiation- | | | |
|---|---|---|---|---|
| | sensitive resin | | LWR | Process |
| | composition | Sensitivity | performance | window |
| Example 21 | R-21 | B | B | B |
| Example 22 | R-22 | A | B | B |
| Example 23 | R-23 | A | B | B |
| Example 24 | R-24 | A | B | B |
| Example 25 | R-25 | A | B | B |
| Example 26 | R-26 | B | B | B |
| Example 27 | R-27 | B | B | B |
| Example 28 | R-28 | A | B | B |
| Example 29 | R-29 | A | B | B |
| Example 30 | R-30 | A | B | B |
| Example 31 | R-31 | A | B | B |
| Example 32 | R-32 | A | B | B |
| Example 33 | R-33 | A | B | B |
| Example 34 | R-34 | A | B | B |
| Example 35 | R-35 | A | B | B |
| Example 36 | R-36 | A | B | B |
| Example 37 | R-37 | A | B | B |
| Example 38 | R-38 | A | B | B |
| Example 39 | R-39 | A | B | B |
| Comparative | CR-3 | C | C | C |
| Example 3 | | | | |

As shown in Table 6, the radiation-sensitive resin compositions of Examples 21 to 39 were found to exhibit high sensitivity, sufficiently wide CD margin, and excellent LWR performance. Among these compositions, compositions were evaluated with a score of sensitivity “A” in the following cases; cases in which the acid diffusion control agents (D-2) and (D-3) each formed of an anion moiety having a structure corresponding to the aforementioned formula (1d) and a cation moiety having a fluorine atom were used as the compound (CD) (Examples 22, 23, and 28 to 39); and cases in which the acid diffusion control agents (D-4) and (D-5) each formed of an anion moiety having a polar group were used (Examples 24 and 25). In contrast, the radiation-sensitive resin composition of Comparative Example 3 exhibited a score of “C” in all measured properties, which was inferior to those of Examples 21 to 39.

1. A radiation-sensitive composition comprising:
a polymer comprising an acid-releasable group; and
a compound represented by formula (1): 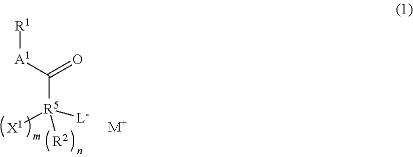
wherein, in the formula (1), R⁵ is a group formed by removing (m+n+2) hydrogen atoms from a monocyclic or condensed cyclic aromatic hydrocarbon ring comprising r carbon atoms; among carbon atoms forming R⁵, the carbon atom to which group —CO-A¹-R¹ is bound and the carbon atom to which L⁻ is bound are located adjacent to each other; L⁻ represents —SO₃⁻ or —COO⁻; and r is an integer of 6 to 14; A¹ represents a single bond, —O—, —S—, or —NR³—; R³ represents a hydrogen atom or a monovalent organic group; R¹ represents a hydrogen atom or a monovalent organic group; X¹ represents a chlorine atom, a bromine atom, or an iodine atom; m is an integer of 1 to (r−2); when m is 2 or greater, a plurality of X¹s are identical to or different from one another; n is an integer of 0 to (r−3); R² represents a substituted or unsubstituted monovalent hydrocarbon group; when n is 2 or greater, a plurality of R²s are identical to or different from one another; m+n is equal to or less than (r−2); and M⁺ represents a monovalent cation.

2. The radiation-sensitive composition according to claim 1, wherein at least one X¹ in the formula (1) is an iodine atom.

3. The radiation-sensitive composition according to claim 1, wherein m in the formula (1) is 2 or greater.

4. The radiation-sensitive composition according to claim 1, wherein m in the formula (1) is 2 or greater, and at least one X¹ is an iodine atom.

5. The radiation-sensitive composition according to claim 1, wherein X¹ in the formula (1) is an iodine atom.

6. The radiation-sensitive composition according to claim 1, wherein M⁺ in the formula (1) comprises a sulfonium cation structure and an iodonium cation structure.

7. The radiation-sensitive composition according to claim 1, wherein the polymer comprising an acid-releasable group comprises an aromatic ring and a hydroxy group which is bound to the aromatic ring.

8. The radiation-sensitive composition according to claim 1, wherein the polymer comprising an acid-releasable group comprises an iodo group.

9. The radiation-sensitive composition according to claim 1, which is suitable for forming a resist pattern through exposure to light of an extreme ultraviolet ray.

10. The radiation-sensitive composition according to claim 1, wherein L⁻ in the formula (1) is —SO₃⁻, and
the composition further comprises a compound that can generate, through exposure to light, an acid having an acidity lower than an acidity of the compound represented by the formula (1).

11. The radiation-sensitive composition according to claim 1, wherein L⁻ in the formula (1) is —COO⁻, and
the composition further comprises a compound that can generate, through exposure to light, an acid having an acidity higher than an acidity of the compound represented by the formula (1).

12. A radiation-sensitive composition comprising a polymer comprising a partial structure represented by formula (2): 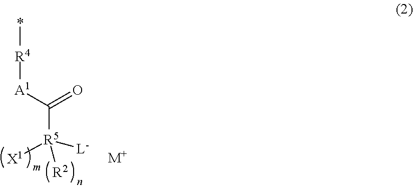
wherein, in the formula (2), R⁵ is a group formed by removing (m+n+2) hydrogen atoms from a monocyclic or condensed cyclic aromatic hydrocarbon ring comprising r carbon atoms; among carbon atoms forming R⁵, the carbon atom to which group —CO-A¹-R¹ is bound and the carbon atom to which L⁻ is bound are located adjacent to each other; L⁻ represents —SO₃⁻ or —COO⁻; and r is an integer of 6 to 14; A¹ represents a single bond, —O—, —S—, or —NR³—; R³ represents a hydrogen atom or a monovalent organic group; R⁴ represents a divalent organic group; X¹ represents a chlorine atom, a bromine atom, or an iodine atom; m is an integer of 1 to (r−2); when m is 2 or greater, a plurality of X¹s are identical to or different from one another; n is an integer of 0 to (r−3); R² represents a substituted or unsubstituted monovalent hydrocarbon group; when n is 2 or greater, a plurality of R²s are identical to or different from one another; m+n is equal to or less than (r−2); * represents a chemical bond; and M⁺ represents a monovalent cation.

13. A method of forming a resist pattern, the method comprising:
forming a resist film on a substrate by use of the radiation-sensitive composition according to claim 1;
exposing the resist film to light; and
developing the resist film which has been exposed to light.

14. The resist pattern formation method according to claim 13, wherein the resist film is exposed to light of an extreme ultraviolet ray.

15. A radiation-sensitive acid-generator represented by formula (1): 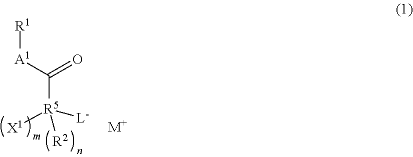
wherein, in the formula (1), R⁵ is a group formed by removing (m+n+2) hydrogen atoms from a monocyclic or condensed cyclic aromatic hydrocarbon ring comprising r carbon atoms; among carbon atoms forming R⁵, the carbon atom to which group —CO-A¹-R¹ is bound and the carbon atom to which L⁻ is bound are located adjacent to each other; L⁻ represents —SO₃⁻ or —COO⁻; and r is an integer of 6 to 14; A¹ represents a single bond, —O—, —S—, or —NR³—; R³ represents a hydrogen atom or a monovalent organic group; R¹ represents a hydrogen atom or a monovalent organic group; X¹ represents a chlorine atom, a bromine atom, or an iodine atom; m is an integer of 1 to (r−2); when m is 2 or greater, a plurality of X¹s are identical to or different from one another; n is an integer of 0 to (r−3); R² represents a substituted or unsubstituted monovalent hydrocarbon group; when n is 2 or greater, a plurality of R²s are identical to or different from one another; m+n is equal to or less than (r−2); and M⁺ represents a monovalent cation.

16. (canceled)
